(19) United States
(12) Patent Application Publication
(10) Pub. No.: US 2005/0090669 A1
(43) Pub. Date: Apr. 28, 2005

(54) Aminoalcohol derivatives as beta-3 adrenergic receptor agonists

(76) Inventors: Kouji Hattori, Osaka (JP); Yasuyo Tomishima, Osaka (JP); Yutaka Nakajima, Osaka (JP); Masashi Imanishi, Osaka (JP)

Correspondence Address: OBLON, SPIVAK, MCCLELLAND, MAIER & NEUSTADT, P.C., 1940 DUKE STREET, ALEXANDRIA, VA, 22314, US

(21) Appl. No.: 10/504,990

(22) PCT Filed: Mar. 10, 2003
(86) PCT No.: PCT/JP03/02821

(30) Foreign Application Priority Data
Mar. 14, 2002 (AU) PS1104
Jan. 10, 2003 (AU) 2003900127

(51) Int. Cl. C07D 49/14; C07D 43/14; C07D 41/14

(52) U.S. Cl. 544/405; 546/193; 546/255; 546/280.4

(57) ABSTRACT

The present invention relates to a compound formula [I] wherein R1 and R⁵ are each independently hydrogen, halogen, lower alkyl, etc., R² is hydrogen or an amino protective group, x is bond, -o-o, —O—CH₂—, etc., y is in which Z is bond, —O—(CH₂)ₘ— (in which m is 1 to 4), etc., R³ is lower alkanoyl, carboxy, lower alkoxycarbonyl, etc., and R⁴ is hydrogen, halogen, hydroxy, phenoxy, lower alkyl, lower alkoxy, etc., and n is 0, 1 or 2, or a salt thereof. The compound [I] of the present invention and pharmaceutically acceptable salts thereof are useful for the prophylactic and/or the therapeutic treatment of pollakiurea or urinary incontinence.

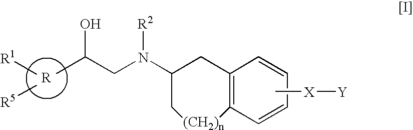

### TECHNICAL FIELD

This invention relates to new aminoalcohol derivatives and salts thereof which are beta-3 (β₃) adrenergic receptor agonists and useful as a medicament.

### DISCLOSURE OF INVENTION

This invention relates to new aminoalcohol derivatives which are β₃ adrenergic receptor agonists and salts thereof.

More particularly, it relates to new aminoalcohol derivatives and salts thereof which have gut sympathomimetic, anti-ulcerous, anti-pancreatitis, lipolytic, anti-urinary incontinence, anti-pollakiuria activities, anti-diabetes and anti-obesity, to processes for the preparation thereof, to a pharmaceutical composition comprising the same and to a method of using the same therapeutically in the treatment and/or prevention of gastro-intestinal disorders caused by smooth muscle contractions in a human being or an animal.

One object of this invention is to provide new and useful aminoalcohol derivatives and salts thereof which have gut sympathomimetic, anti-ulcerous, lipolytic, anti-urinary incontinence, anti-pollakiuria activities, anti-diabetes and anti-obesity.

Another object of this invention is to provide processes for the preparation of said aminoalcohol derivatives and salts thereof.

A further object of this invention is to provide a pharmaceutical composition comprising, as an active ingredient, said aminoacohol derivatives and salts thereof.

Still further object of this invention is to provide a therapeutical method for the treatment and/or prevention of aforesaid diseases in a human being or an animal, using said aminoalcohol derivatives and salts thereof.

The object aminoalcohol derivatives of this invention are new and can be represented by compound of the following formula [I]: 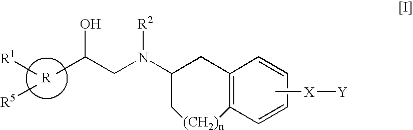
wherein 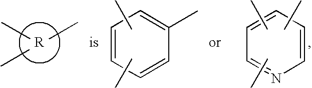
R¹ and R⁵ are each independently hydrogen, halogen lower alkyl, mono(or di or tri)halo(lower)alkyl or cyano,
R² is hydrogen or an amino protective group,
X is bond, —O—, —O—CH₂—, 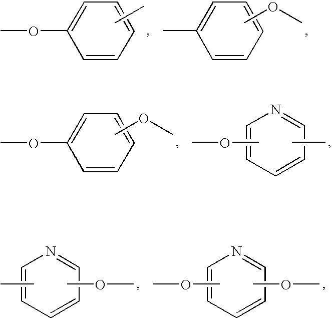
   —(CH₂)_{q}— (in which q is 1 to 3),
   —CH═CH—, —C≡C—, —NH—, —S— or —SO₂—,
Y is 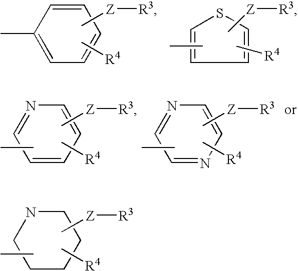
   in which Z is bond, —O—(CH₂)ₘ— (in which m is 1 to 4), lower alkylene or lower alkenylene,
      R³ is lower alkanoyl, carboxy, lower alkoxycarbonyl, carbamoyl, (lower alkylsulfonyl)carbamoyl, (phenylsulfonyl)carbamoyl, (benzylsulfonyl)carbamoyl or tetrazolyl, and
      R⁴ is hydrogen, halogen, hydroxy, phenoxy, lower alkyl, lower alkoxy, cyclo(lower)alkyloxy, 3,4,5,6-tetrahydro-2H-pyranyloxy, phenoxy, nitro, cyano or 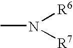
      in which
         R⁶ is hydrogen or lower alkyl, and
         R⁷ is hydrogen, lower alkyl, lower alkanoyl, lower alkoxycarbonyl, benzyloxycarbonyl, benzoyl, furoyl, lower alkylcarbanoyl, phenylcarbamoyl, lower alkylsulfonyl, 3,4,5,6-tetrahydro-2H-pyranyl or phenyl, or
         R⁶ and R⁷ are combined to form pyrrolidino or piperidino together with the nitrogen atom which may be substituted with oxo, and
n is 0, 1 or 2,
   or a salt thereof.

According to this invention, the object compounds can be prepared by processes which are illustrated in the following schemes. 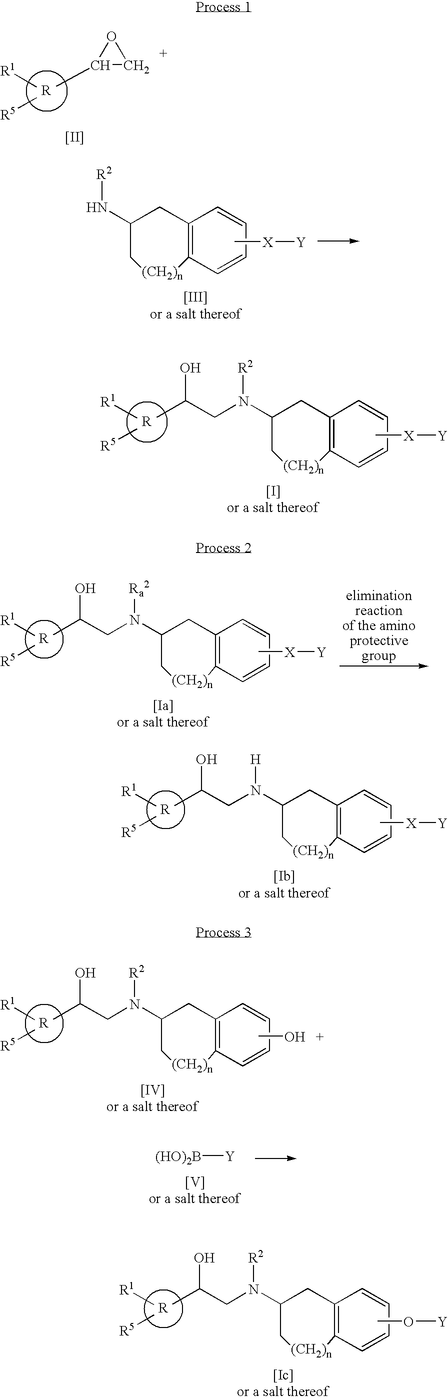 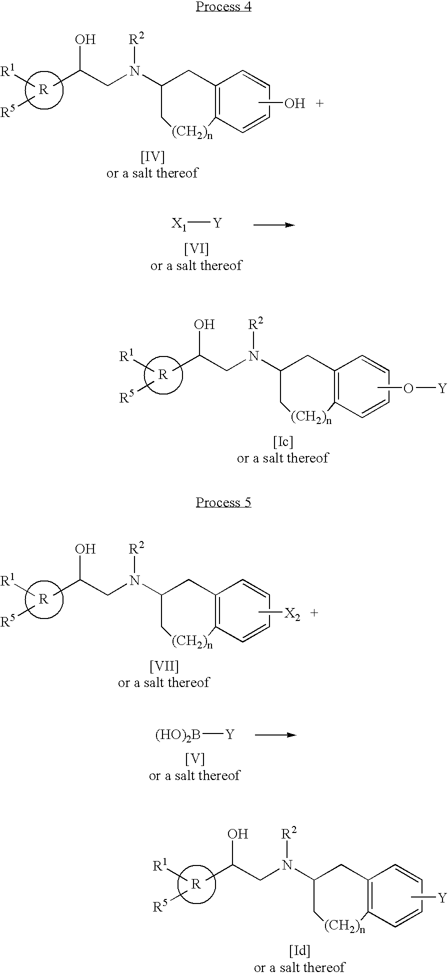
wherein 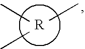
R¹, R², R⁵, X, Y and n are each as defined above,
   Rₐ² is an amino protective group, and
   X₁ and X₂ are each a leaving group.

As to the starting compounds [II], [III], [Ia], [IV], [V], [VI] and [VII], some of them are novel and can be prepared by the procedures described in the Preparations and Examples mentioned below or a conventional manner.

In the above and subsequent description of the present specification, suitable examples of the various definition to be included within the scope of the invention are explained in detail in the following.

The term “lower” is intended to mean a group having 1 to 6, preferably 1 to 4, carbon atom(s), unless otherwise indicated.

Suitable “lower alkylene” is straight or branched one having 1 to 6 carbon atom(s) and may include methylene, ethylene, trimethylene, propylene, tetramethylene, methylmethylene, methyltrimethylene, hexamethylene, and the like.

Suitable example of “lower alkyl” and “lower alkyl” moiety in the terms of “(lower alkylsulfonyl)carbamoyl”, “mono(or di or tri)halo(lower)alkyl”, etc. may include straight or branched one having 1 to 6 carbon atom(s), such as methyl, ethyl propyl, isopropyl, butyl, isobutyl, sec-butyl, tert-butyl, pentyl, 1-methylpentyl, tert-pentyl, neopentyl, hexyl, isohexyl, and the like, in which preferable one is methyl.

Suitable “cyclo(lower)alkyl” moiety in the term of “cyclo(lower)alkyloxy” may include cyclopropyl, cyclobutyl, cyclopentyl, and cyclohexyl, in which the preferred one may be cyclohexyl.

The term “lower alkenylene” means one having one or two double bond(s) in the straight or branched lower alkylene group as defined above.

Suitable “lower alkenylene” may include one having 2 to 6 carbon atoms such as vinylene, 1-propenylene, 2-propenylene, 1,3-butadienylene, 1-methylvinylene and the like.

Suitable “lower alkoxy” and “lower alkoxy” moiety in the term of “lower alkoxycarbonyl” may include methoxy, ethoxy, propoxy, isopropoxy, butoxy, iso-butoxy, tert-butoxy, pentyloxy, tert-pentyloxy, hexyloxy and the like, in which preferable one is methoxy or ethoxy.

Suitable “lower alkanoyl” may include formyl, acetyl, propanoyl, butanoyl, 2-methylpropanoyl, pentanoyl, 2,2-dimethylpropanoyl, hexanoyl and the like, in which preferable one is formyl.

Suitable “halogen” may be fluoro, chloro, bromo and iodo, in which preferable one is chloro.

Suitable “mono(or di or tri)halo(lower)alkyl” may be fluoromethyl, difluoromethyl, trifluoromethyl, chloromethyl, dichloromethyl, trichloromethyl, bromomethyl, dibromomethyl, tribromomethyl, 1 or 2-fluoroethyl, 1 or 2-bromoethyl, 1 or 2-chloroethyl, 1,1-difluoroethyl, 2,2-difluoroethyl, and the like, in which the preferred one may be trifluoromethyl.

Suitable “leaving group” may include hydroxy, reactive group derived from hydroxy and the like.

Suitable “reactive group derived from hydroxy” may include acid residue and the like.

Suitable “acid residue” may include halogen (e.g. fluoro, chloro, bromo, iodo), acyloxy (e.g. acetoxy, tosyloxy, mesyloxy, trifluoromethanesulfonyloxy, etc.) and the like.

Suitable example of “amino protective group” moiety may be common amino protective group such as substituted or unsubstituted lower alkanoyl [e.g. formyl, acetyl, propionyl, trifluoroacetyl, etc.], phthaloyl, lower alkoxycarbonyl [e.g. tert-butoxycarbonyl, tert-amyloxycarbonyl, etc.], substituted or unsubstituted aralkyloxycarbonyl [e.g. benzyloxycarbonyl, p-nitrobenzyloxycarbonyl, etc.], substituted or unsubstituted arenesulfonyl [e.g. benzenesulfonyl, tosyl, etc.], nitrophenylsulfenyl, ar(lower)alkyl [e.g. trityl, benzyl, etc.], and the like, in which preferable one is tert-butoxycarbonyl.

Suitable salts of the object aminoalcohol derivative [I] are pharmaceutically acceptable salts and include conventional non-toxic salts such as an inorganic acid addition salt [e.g. hydrochloride, hydrobromide, sulfate, phosphate, etc.], an organic acid addition salt [e.g. formate, acetate, trifluoroacetate, oxalate, maleate, fumarate, tartrate, citrate, methanesulfonate, benzenesulfonate, toluenesulfonate, etc., an alkali metal salt [e.g. sodium salt, potassium salt, etc.] or the like.

The Processes 1 to 5 for preparing the object compounds of the present invention are explained in detail in the following.

Process 1

The object compound [I] or a salt thereof can be prepared by reacting a compound [II] with a compound [III] or a salt thereof.

Suitable salt of the compound [III] may be the same as those exemplified for the compound [I].

The reaction is preferably carried out in the presence of a base such as an alkali metal carbonate [e.g. sodium carbonate, potassium carbonate, etc.], an alkaline earth metal carbonate [e.g. magnesium carbonate, calcium carbonate, etc.], an alkali metal bicarbonate [e.g. sodium bicarbonate, potassium bicarbonate, etc.], tri(lower)alkylamine [e.g. trimethylamine, triethylamine, etc.], picoline or the like.

The reaction is usually carried out in a conventional solvent, such as an alcohol [e.g. methanol, ethanol, propanol, isopropanol, etc.), diethyl ether, tetrahydrofuran, dioxane, or any other organic solvent which does not adversely influence the reaction.

The reaction temperature is not critical, and the reaction can be carried out under cooling to heating.

Process 2

The object compound [Ib] or a salt thereof can be prepared by subjecting a compound [Ia] or a salt thereof to elimination reaction of the amino protective group.

Suitable salts of the compounds [Ia] and [Ib] may be the same as those exemplified for the compound [I].

This reaction can be carried out in a similar manner to that of Example 2 or 9 mentioned below.

Process 3

The object compound [Ic] or a salt thereof can be prepared by reacting a compound [IV] or a salt thereof with a compound [V] or a salt thereof.

Suitable salts of the compounds [Ic], [IV] and [V] may be the same as those exemplified for the compound [I].

This reaction can be carried out in a similar manner to that of Examples 1 mentioned below.

Process 4

The object compound [Ic] or a salt thereof can be prepared by reacting a compound [IV] or a salt thereof with a compound [VI] or a salt thereof.

Suitable salts of the compound [Ic], [IV] and [VI] may be the same as those exemplified for the compound [I].

This reaction can be carried out in a similar manner to that of Example 7 mentioned below.

Process 5

The object compound [Id] or a salt thereof can be prepared by reacting a compound [VII] or a salt thereof with a compound [V] or a salt thereof.

Suitable salts of the compounds [Id], [VII] and [V] may be the same as those exemplified for the compound [I].

This reaction can be carried out in a similar manner to that of Example 15 mentioned below.

The compounds obtained by the above processes can be isolated and purified by a conventional method such as pulverization, recrystallization, column chromatography, reprecipitation, or the like, and converted to the desired salt in conventional manners, if necessary.

It is to be noted that the compound [I] and the other compounds may include one or more stereoisomers due to asymmetric carbon atoms, and all of such isomers and mixture thereof are included within the scope of this invention.

It is further to be noted that isomerization or rearrangement of the object compound [I] may occur due to the effect of the light, acid base or the like, and the compound obtained as the result of said isomerization or rearrangement if also included within the scope of the present invention.

It is also to be noted that the solvating form of the compound (I] (e.g. hydrate, etc.) and any form of the crystal of the compound [I] are included within the scope of the present invention.

The object compound [I] or a salt thereof possesses gut sympathomimetic, anti-ulcerous, anti-pancreatitis, lipolytic, anti-urinary incontinence and anti-pollakiuria activities, and are useful for the treatment and/or prevention of gastro-intestinal disorders caused by smooth muscle contractions in human beings or animals, and more parcitularly for the treatment and/or prevention of spasm or hyperanakinesia in case of irritable bowel syndrome, gastritis, gastric ulcer, duodenal ulcer, enteritis, cholecystopathy, cholantitis, urinary calculus and the like; for the treatment and/or prevention of ulcer such as gastric ulcer, duodenal ulcer, peptic ulcer, ulcer causes by non steroidal anti-inflammatory drags, or the like; for the treatment and/or prevention of dysuria such as pollakiuria, urinary incontinence or the like in case of nervous pollakiuria, neurogenic bladder dysfunction, nocturia, unstable bladder, cystospasm, chronic cystitis, chronic prostatitis, prostatic hypertrophy or the like; for the treatment and/or prevention of pancreatitis, obesity, diabetes, glycosuria, hyperlipidemia, hypertension, atherosclerosis, glaucoma, melancholia, depression or the like; for the treatment and/or prevention of diseases as the result of insulin resistance (e.g. hypertension, hyperinsulinemia, etc.); for the treatment and/or prevention of neurogenetic inflammation; and for reducing a wasting condition, and the like.

Additionally, β₃ adrenergic receptor agonists are known to lower triglyceride and cholesterol levels and to raise high density lipoprotein levels in mammals (U.S. Pat. No. 5,451,677). Accordingly, the object compound [I] in useful in the treatment and/or prevention of conditions such as hyper-triglyceridaemia, hypercholesterolaemia and in lowering high density lipoprotein levels as well as in the treatment of atherosclerotic and cardiovascular diseases and relates conditions.

Moreover, the object compound [I] is useful for inhibiting uterine contractions, preventing premature labor, and treating and preventing dysmenorrhea.

In order to show the usefulness of the compound [I] for the prophylactic and therapeutic treatment of abovementioned disease in human being or animals, a representative compound of the compound [I] was tested on the following pharmaceutical test.

Test

Effect on the Increase in Intravesical Pressure Induced by Carbachol in Anesthetized Dog

Test Compound

### (1) 5-[[(7S)-7-[[(2R)-2-(4-Chlorophenyl)-2-hydroxyethyl]-amino]-5,6,7,8-tetrahydro-2-naphthalenyl]oxy]-2-methoxybenzoic Acid Hydrochloride (Compound of Example 38-(9))

Test Method

Female Beagle dogs weighing 8.0-15.0 kg were fasted for 24 hours and maintained under halothane anesthesia. A 12 F Foley catheter was lubricated with water soluble jelly, inserted into the urethral orifice and advanced approximately 10 cm until the balloon tip was placed well inside the bladder. The balloon was then inflated with 5 ml of room air and catheter slowly withdrawn just part the first resistance that is felt at the bladder neck. Urine was completely drained out through the catheter, and 30 ml of biological saline was infused. The catheter was connected to pressure transducer, and intravesical pressure (IVP) was continuously recordered. The test compound was administered intravenously at 30 minutes before the administration of carbachol (1.8 μg/kg). Percent inhibition of IVP increase by test compound was calculated by dividing IVPa (IVP increase induced by carbachol after test compound administaration) by IVPb (IVP increase induced by carbachol just before test compound administration).

Test Results

| Treatment | Percent inhibition of IVP increase |
|---|---|
| Test Compound (1) | 54 |
| (0.032 mg/kg) | |

Preferred embodiments of the object compound [I] are as follows:
R¹ and R⁵ are each independently hydrogen, halogen (more preferably chloro or fluoro, most preferably chloro), lower alkyl (more preferably C₁-C₄ alkyl, most preferably methyl) or mono(or di or tri)halo(lower)alkyl (more preferably mono(or di or tri)halo(C₁-C₄)alkyl, most preferably trifluoromethyl),
R² is hydrogen,
X is bond, —O—, —O—CH₂—, 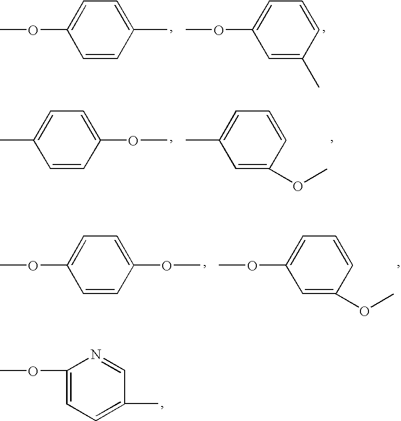
   (CH₂)_{q}— (in which q is 1 or 2),
   —CH═CH—, —C≡C—, —NH—, —S— or —SO₂—,
Y is 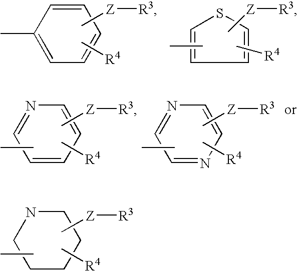
   in which Z is bond, —O—(CH₂)ₘ— (in which m is 1 to 4), lower alkylene (more preferably C₁-C₄ alkylene, most preferably methylene) or lower alkenylene (more preferably C₂-C₄ alkenylene, most preferably vinylene),
      R³ is lower alkanoyl (more preferably C₁-C₄ alkanoyl, most preferably formyl), carboxy, lower alkoxycarbonyl (more preferably C₁-C₄ alkoxycarbonyl, most preferably methoxycarbonyl or ethoxycarbonyl), carbamoyl, (lower alkylsulfonyl)carbamoyl (more preferably C₁-C₄ alkylsulfonyl)carbamoyl, most preferably (methylsulfonyl)carbamoyl), (phenylsulfonyl)carbamoyl, (benzylsulfonyl)carbamoyl or tetrazolyl, and
      R⁴ is hydrogen, halogen (more preferably chloro or fluoro, most preferably chloro), hydroxy, phenoxy, lower alkyl (more preferably C₁-C₄ alkyl, most preferably methyl), lower alkoxy (more preferably C₁-C₄ alkoxy, most preferably methoxy), cyclo(lower)alkyloxy (more preferably cyclo(C₃-C₆)alkyloxy, most preferably cyclohexyloxy), 3,4,5,6-tetrahydro-2H-pyranyloxy (more preferably 3,4,5,6-tetrahydro-2H-pyran-4-yloxy), phenoxy, nitro, cyano or 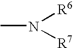
      in which
         R⁶ is hydrogen or lower alkyl (more preferably C₁-C₄ alkyl, most preferably methyl), and
         R⁷ is hydrogen, lower alkyl (more preferably C₁-C₄ alkyl, most preferably methyl), lower alkanoyl (more preferably C₁-C₄ alkanoyl, most preferably acetyl), lower alkoxycarbonyl (more preferably C₁-C₄ alkoxycarbonyl, most preferably methoxycarbonyl), benzyloxycarbonyl, benzoyl, furoyl, lower alkylcarbamoyl (more preferably C₁-C₄ alkylcarbomoyl, most preferably methylcarbamoyl), phenylcarbamoyl, lower alkylsulfonyl (more preferably C₁-C₄ alkylsulfonyl, most preferably methylsulfonyl), 3,4,5,6-tetrahydro-2H-pyranyl (more preferably 3,4,5,6-tetrahydro-2H-pyran-4-yl) or phenyl, or
         R⁶ and R⁷ are combined to form pyrrolidino or piperidino together with the nitrogen atom which may be substituted with oxo, and
n is 0, 1 or 2.

More preferred embodiments of the object compound [I] are as follows: 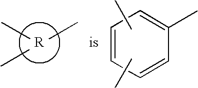
R¹ is halogen (more preferably chloro),
R⁵ is hydrogen,
R² is hydrogen,
X is bond, —O— or —O—CH₂—,
Y is 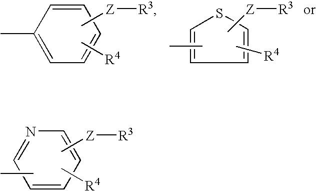
   in which Z is bond, —O—(CH₂)ₘ— (in which m is 1 or 2) or lower alkenylene (more preferably C₂-C₄ alkenylene, most preferably vinylene),
      R³ is lower alkanoyl (more preferably C₁-C₄ alkanoyl, most preferably formyl), carboxy, lower alkoxycarbonyl (more preferably C₁-C₄ alkoxycarbonyl, most preferably methoxycarbonyl or ethoxycarbonyl), carbamoyl or tetrazolyl, and
      R⁴ is hydrogen or lower alkoxy (more preferably C₁-C₄ alkoxy, most preferably methoxy), and
n is 1 or 2.

More preferred embodiments of the object compound [I] are as follows. 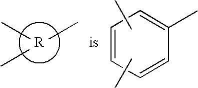
R¹ is chloro,
R⁵ is hydrogen,
R² is hydrogen,
X is bond or —O—,
Y is 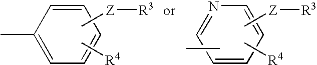
   in which Z is bond or lower alkenylene (more preferably C₂-C₄ alkenylene, most preferably vinylene),
      R³ is carboxy, and
      R⁴ is hydrogen or lower alkoxy (more preferably C₁-C₄ alkoxy, most preferably methoxy), and
n is 1.

The following Preparations and Examples are given for the purpose of illustrating this invention.

Preparation 1

To a mixture of (7S)-7-[[(2R)-2-(3-chlorophenyl)-2-hydroxyethyl]amino]-2-hydroxy-5,6,7,8-tetrahydronaphthalene (10 g) in tetrahydrofuran (100 ml) was added di-tert-butyl dicarbonate (8 g) at room temperature, and the mixture was stirred at the same temperature for 12 hours. The resulting mixture was evaporated under reduced pressure and the residue was purified by column chromatography on silica gel to give (7S)-7-[N-[(2R)-2-(3-chlorophenyl)-2-hydroxyethyl]-N-(tert-butoxycarbonyl)amino]-2-hydroxy-5,6,7,8-tetrahydronaphthalene (12 g).

¹H NMR (200 MHz, CDCl₃, δ): 1.51 (9H, s), 1.7-1.9 (2H, m), 2.7-3.0 (4H, m), 3.2-3.4 (1H, m), 3.4-3.7 (1H, m), 4.0-4.2 (1H, m), 4.7-4.9 (1H, m), 6.03 (1H, br.s), 6.5-6.6 (2H, m), 6.62 (1H, dd, J=2.4, 8.4 Hz), 6.90 (1H, d, J=8.4 Hz), 7.3-7.5 (3H, m), 7.37 (1H, s)

Ms: 440 (M+22)

Preparation 2

The following compound was obtained according to a similar manner to that of Preparation 1.

### (8S)-8-[N-[(2R)-2-(3-Chlorophenyl)-2-hydroxyethyl]-N-(tert-butoxycarbonyl)amino]-6,7,8,9-tetrahydro-5H-benzo[a][7]annulen-2-ol

¹H NMR (200 MHz, CDCl₃, δ): 1.50 (9H, s), 1.4-2.0 (4H, m), 2.6-2.8 (3H, m), 3.1-3.5 (4H, m), 4.8-5.0 (1H, m), 6.03 (1H, br.s), 6.58 (2H, m), 6.92 (1H, m), 7.26 (3H, m), 7.41 (1H, s)

Ms: 454 (M+22)

### EXAMPLE 1

To a mixture of (7S)-7-[N-[(2R)-2-(3-chlorophenyl)-2-hydroxyethyl]-N-(tert-butoxycarbonyl)amino]-2-hydroxy-5,6,7,8-tetrahydronaphthalene (400 mg) in dichlorometane (10 ml) and triethylamine (1 ml) were added (3-methoxycarbonylphenyl)boronic acid (400 mg) and copper(II) acetate (400 mg) and molecular sieves 4A (1 g) at room temperature, and the mixture was stirred at the same temperature for 12 hours. The resulting mixture was filtrated by celite and the mother layer was evaporated under reduced pressure. The residue was purified by column chromatography on silica gel to give 3-[([(7S)-7-[N-[(2R)-2-(3-chlorophenyl)-2-hydroxyethyl]-N-(tert-butoxycarbonyl)-amino]-5,6,7,8-tetrahydro-2-naphthalenyl]oxy]benzoic acid methyl ester (240 mg).

¹H NMR (200 MHz, CDCl₃, δ): 1.51 (9H, s), 1.7-1.9 (2H, m), 2.7-3.0 (4H, m), 3.2-3.4 (1H, m), 3.4-3.7 (1H, m), 3.90 (3H, s), 4.0-4.2 (1H, m), 4.8-5.0 (1H, m), 6.6-6.9 (2H, m), 7.05 (1H, d, J=8.4 Hz), 7.1-7.8 (8H, m)

Ms: 574 (M+22)

### EXAMPLE 2

To a solution of 3-[[(7S)-7-[N-[(2R)-2-(3-chlorophenyl)-2-hydroxyethyl]-N-(tert-butoxycarbonyl)-amino]-5,6,7,8-tetrahydro-2-naphthalenyl]oxy]benzoic acid methyl ester (240 mg) in methanol (10 ml) was added 1N sodium hydroxide (5 ml) at room temperature, and the mixture was stirred at the same temperature for 12 hours. The resulting mixture was evaporated under reduced pressure. The residue was diluted with a mixture of ethyl acetate (30 ml) and 1N hydrochloric acid (10 ml), and the organic layer was washed with brine, dried over magnesium sulfate, and evaporated under reduced pressure. The obtained benzoic acid was diluted with 6N hydrogen chloride in dioxane (10 ml) and the mixture was allowed to keep at room temperature for 4 hours. The mixture was evaporated under reduced pressure and the obtained solid was washed with ethyl ether to give 3-[[(7S)-7-[[(2R)-2-(3-chlorophenyl)-2-hydroxyethyl]amino]-5,6,7,8-tetrahydro-2-naphthalenyl]oxy]benzoic acid hydrochloride (100 mg).

¹H NMR (200 MHz, DMSO-d₆, δ): 1.7-2.0 (1H, m), 2.1-2.3 (1H, m), 2.7-3.5 (7H, m), 5.0-5.1 (1H, m), 6.4 (br.s), 6.8-7.0 (2H, m), 7.1-7.8 (9H, m)

Ms: 438 (M+1)

### EXAMPLE 3

The following compounds were obtained according to a similar manner to that of Example 1.

### (1) 4-[[(7S)-7-[N-[(2R)-2-(3-Chlorophenyl)-2-hydroxyethyl]-N-(tert-butoxycarbonyl)amino]-5,6,7,8-tetrahydro-2-naphthalenyl]oxy]benzoic Acid Methyl Ester

¹H NMR (200 MHz, CDCl₃, δ): 1.51 (9H, s), 1.7-1.9 (2H, m), 2.7-3.0 (4H, m), 3.2-3.4 (1H, m), 3.4-3.7 (1H, m), 3.89 (3H, s), 4.0-4.2 (1H, m), 4.8-5.0 (1H, m), 6.7-7.3 (8H, m), 7.39 (1H, s), 7.99 (2H, d, J=8.6 Hz)

Ms: 574 (M+22)

### (2) [3-[[(7S)-7-[N-[(2R)-2-(3-Chlorophenyl)-2-hydroxyethyl]-N-(tert-butoxycarbonyl)amino]-5,6,7,8-tetrahydro-2-naphthalenyl]oxy]phenoxy](tert-butyl)dimethylsilane

¹H NMR (200 MHz, CDCl₃, δ): 0.17 (6H, s), 0.95 (9H, s), 1.51 (9H, s), 1.7-1.9 (2H, m), 2.7-3.0 (4H, m), 3.2-3.4 (1H, m), 3.4-3.7 (1H, m), 4.0-4.2 (1H, m), 4.8-5.0 (1H, m), 6.4-6.9 (5H, m), 7.0-7.5 (6H, m)

Ms: 646 (M+22)

### (3) [4-[[(7S)-7-[N-[(2R)-2-(3-Chlorophenyl)-2-hydroxyethyl]-N-(tert-butoxycarbonyl)amino]-5,6,7,8-tetrahydro-2-naphthalenyl]oxy]phenoxy](tert-butyl)-dimethylsilane

¹H NMR (200 MHz, CDCl₃, δ): 0.17 (6H, s), 0.95 (9H, s), 1.51 (9H, s), 1.7-1.9 (2H, m), 2.7-3.0 (4H, m), 3.2-3.4 (1H, m), 3.4-3.7 (1H, m), 4.0-4.2 (1H, m), 4.8-5.0 (1H, m), 6.5-7.0 (6H, m), 7.2-7.4 (5H, m)

Ms: 646 (M+22)

### (4) 3-[[(8S)-8-[N-[(2R)-2-(3-Chlorophenyl)-2-hydroxyethyl]-N-(tert-butoxycarbonyl)amino]-6,7,8,9-tetrahydro-5H-benzo[a]cyclohepten-2-yl]oxy]benzoic Acid Methyl Ester

¹H NMR (200 MHz, CDCl₃, δ): 1.51 (9H, s), 1.8-2.1 (2H, m), 2.5-2.8 (2H, m), 3.0-3.4 (3H, m), 3.91 (3H, s), 4.91 (1H, m), 6.6-6.8 (1H, m), 6.9-7.1 (1H, m), 7.1-7.8 (9H, m)

Ms: 588 (M+22)

### (5) 4-[[(8S)-8-[N-[(2R)-2-(3-Chlorophenyl)-2-hydroxyethyl]-N-(tert-butoxycarbonyl)amino]-6,7,8,9-tetrahydro-5H-benzo[a]cyclohepten-2-yl]oxy]benzoic acid methyl ester

¹H NMR (200 MHz, CDCl₃, δ): 1.51 (9H, s), 1.8-2.1 (2H, m), 2.5-2.8 (2H, m), 3.0-3.4 (3H, m), 3.91 (3H, s). 4.91 (1H, m), 6.9-7.8 (11H, m)

Ms: 588 (M+22)

### EXAMPLE 4

The following compounds were obtained according to a similar manner to that of Example 2.

### (1) 4-[[(7S)-7-[[(2R)-2-(3-Chlorophenyl)-2-hydroxyethyl]-amino]-5,6,7,8-tetrahydro-2-naphthalenyl]oxy]benzoic acid hydrochloride

¹H NMR (200 MHz, DMSO-d₆, δ): 1.7-2.0 (1H, m), 2.1-2.3 (1H, m), 2.7-3.5 (7H, m), 5.0-5.1 (1H, m), 6.4 (br.s), 6.7-6.9 (2H, m), 6.99 (2H, d, J=8.6 Hz), 7.19 (1H, d, J=8.4 Hz), 7.2-7.5 (4H, m), 7.93 (2H, d, J=8.6 Hz)

Ms: 438 (M+1)

### (2) [3-[[(7S)-7-[[(2R)-2-(3-Chlorophenyl)-2-hydroxyethyl]-amino]-5,6,7,8-tetrahydro-2-naphthalenyl]oxy]phenoxy]-acetic Acid Hydrochloride

¹H NMR (200 MHz, DMSO-d₆, δ): 1.7-2.0 (1H, m), 2.2-2.5 (1H, m), 2.6-3.6 (7H, m), 4.65 (2H, s), 5.07 (1H, m), 6.36 (1H, m), 6.5-6.8 (5H, m), 7.0-7.6 (6H, m), 8.97 (1H, m), 9.44 (1H, m)

Ms: 468 (M+1)

### (3) [4-[[(7S)-7-[[(2R)-2-(3-Chlorophenyl)-2-hydroxyethyl]-amino]-5,6,7,8-tetrahydro-2-naphthalenyl]oxy)phenoxy]-acetic Acid Hydrochloride

¹H NMR (200 MHz, DMSO-d₆, δ): 1.7-2.0 (1H, m), 2.2-2.5 (1H, m), 2.6-3.6 (7H, m), 4.55 (2H, s), 5.04 (1H, m), 6.37 (1H, m), 6.6-7.0 (7H, m), 7.3-7.5 (4H, m)

Ms: 468 (M+1)

### (4) 6-[[(7S)-7-[[(2R)-2-(3-Chlorophenyl)-2-hydroxyethyl]-amino]-5,6,7,8-tetrahydro-2-naphthalenyl]oxy]nicotinic Acid Hydrochloride

¹H NMR (200 MHz, DMSO-d₆, δ): 1.7-2.0 (1H, m), 2.3-2.5 (1H, m), 2.7-3.7 (7H, m), 5.12 (1H, m), 6.8-7.0 (2H, m), 7.0-7.3 (2H, m), 7.4-7.6 (4H, m), 8.27 (1H, dd, J=2.2, 8.6 Hz), 8.64 (1H, d, J=2.2 Hz), 9.0 (1H, br.s), 9.6 (1H, br.s)

Ms: 439 (M+1)

### (5) 3-[(7S)-7-[[(2R)-2-(3-Chlorophenyl)-2-hydroxyethyl]-amino]-5,6,7,8-tetrahydro-2-naphthalenyl]benzoic Acid Hydrochloride

¹H NMR (200 MHz, DMSO-d₆, δ): 1.7-2.0 (1H, m), 2.1-2.3 (1H, m), 2.5-3.7 (7H, m), 5.07 (1H, m), 6.4 (1H, m), 7.24 (1H, d, J=8.0 Hz), 7.3-7.7 (7H, m), 7.90 (2H, m), 8.16 (1H, s), 8.94 (1H, m), 9.28 (1H, m)

Ms: 422 (M+1)

### (6) 4-[(7S)-7-[[(2R)-2-(3-Chlorophenyl)-2-hydroxyethyl]-amino]-5,6,7,8-tetrahydro-2-naphthalenyl]benzoic Acid Hydrochloride

¹H NMR (200 MHz, DMSO-d₆, δ): 1.7-2.0 (1H, m), 2.1-2.3 (1H, m), 2.5-3.7 (7H, m), 5.07 (1H, m), 6.38 (1H, m), 7.24 (1H, d, J=8.0 Hz), 7.3-7.6 (6H, m), 7.76 (2H, d, J=8.4 Hz), 8.01 (2H, d, J=8.4 Hz)

Ms: 422 (M+1)

### (7) [3-[(7S)-7-[[(2R)-2-(3-Chlorophenyl)-2-hydroxyethyl]-amino]-5,6,7,8-tetrahydro-2-naphthalenyl]phenoxy]acetic Acid Hydrochloride

¹H NMR (200 MHz, DMSO-d₆, δ): 1.7-2.0 (1H, m), 2.1-2.3 (1H, m), 2.5-3.7 (7H, m), 4.79 (2H, s), 5.05 (1H, m), 6.38 (1H, m), 6.89 (1H, dd, J=8.4, 2.2 Hz), 7.0-7.4 (10H, m)

Ms: 452 (M+1)

### (8) (4-[(7S)-7-[[(2R)-2-(3-Chlorophenyl)-2-hydroxyethyl]-amino]-5,6,7,8-tetrahydro-2-naphthalenyl]phenoxy]acetic Acid Hydrochloride

¹H NMR (200 MHz, DMSO-d₆, δ): 1.7-2.0 (1H, m), 2.1-2.3 (1H, m), 2.5-3.7 (7H, m), 4.71 (2H, s), 5.08 (1H, m), 6.38 (1H, m), 6.98 (2H, d, J=8.4 Hz), 7.09 (1H, d, J=8.4 Hz), 7.2-7.7 (8H, m), 8.97 (1H, m), 9.41 (1H, m)

Ms: 452 (M+1)

### (9) 3-[[(8S)-8-[[(2R)-2-(3-Chlorophenyl)-2-hydroxyethyl]-amino]-6,7,8,9-tetrahydro-5H-benzo[a]cyclohepten-2-yl]oxy]benzoic Acid Hydrochloride

¹H NMR (200 MHz, DMSO-d₆, δ): 1.2-1.4 (1H, m), 1.7-2.1 (2H, m), 2.2-2.3 (1H, m), 2.7-3.4 (7H, m), 4.99 (1H, m), 6.32 (1H, br.s), 6.85 (1H, dd, J=2.4, 8.0 Hz), 7.01 (1H, d, J=2.4 Hz), 7.1-7.6 (8H, m), 7.68 (1H, d, J=8 Hz)

Ms: 452 (M+1)

### (10) 4-[[(8S)-8-[[(2R)-2-(3-Chlorophenyl)-2-hydroxyethyl]-amino]-6,7,8,9-tetrahydro-5H-benzo[a]cyclohepten-2-yl]oxy]benzoic Acid Hydrochloride

¹H NMR (200 MHz, DMSO-d₆, δ): 1.2-1.4 (1H, m), 1.7-2.3 (3H, m), 2.7-3.4 (7H, m), 5.0 (1H, m), 6.32 (1H, s), 6.9-7.4 (9H, m), 7.93 (2H, d, J=8 Hz)

Ms: 452 (M+1)

### EXAMPLE 5

To a solution of [3-[[(7S)-7-[N-[(2R)-2-(3-chlorophenyl)-2-hydroxyethyl]-N-(tert-butoxycarbonyl)amino]-5,6,7,8-tetrahydro-2-naphthalenyl]oxy]phenoxy](tert-butyl)dimethylsilane (600 mg) in tetrahydrofuran (20 ml) was added tetrabutylammonium fluoride (5 ml, 1M solution in tetrahydrofuran) at room temperature and stirred for 3 hours. The mixture was poured into a mixture of water and ethyl acetate and the organic layer was washed with 1N hydrochloric acid and brine respectively, then dried over magnesium sulfate. After filtration, the solvent was evaporated, and the residue was diluted in N,N-dimethylformamide (10 ml). To the solution were added potassium carbonate (1 g) and ethyl bromoacetate (0.5 ml) at room temperature and stirred for 4 hours. The mixture was poured into a mixture of water and ethyl acetate and the organic layer was washed with 1N hydrochloric acid and brine respectively, then dried over magnesium sulfate. After filtration, the solvent was evaporated, and the obtained residue was purified by column chromatography on silica gel to give [3-[[(7S)-7-[N-[(2R)-2-(3-chlorophenyl)-2-hydroxyethyl]-N-(tert-butoxycarbonyl)amino]-5,6,7,8-tetrahydro-2-naphthalenyl]oxy]phenoxy]acetic acid ethyl ester (450 mg).

¹H NMR (200 MHz, CDCl₃, δ): 1.25 (3H, t, J=6.8 Hz), 1.51 (9H, s), 1.7-1.9 (2H, m), 2.7-3.0 (4H, m), 3.2-3.4 (1H, m), 3.4-3.7 (1H, m), 4.0-4.2 (1H, m), 4.21 (2H, q, J=6.8 Hz), 4.58 (2H, s), 4.8-5.0 (1H, m), 6.5-6.9 (5H, m), 7.0-7.5 (6H, m)

Ms: 618 (M+22)

### EXAMPLE 6

The following compounds were obtained according to a similar manner to that of Example 5.

### (1) [4-[[(7S)-7-[N-[(2R)-2-(3-Chlorophenyl)-2-hydroxyethyl]-N-(tert-butoxycarbonyl)amino]-5,6,7,8-tetrahydro-2-naphthalenyl]oxy]phenoxy]acetic Acid Ethyl Ester

¹H NMR (200 MHz, CDCl₃, δ): 1.25 (3H, t, J=6.8 Hz), 1.51 (9H, s), 1.7-1.9 (2H, m), 2.7-3.0 (4H, m), 3.2-3.4 (1H, m), 3.4-3.7 (1H, m), 4.0-4.2 (1H, m), 4.21 (2H, q, J=6.8 Hz), 4.58 (2H, s), 4.8-5.0 (1H, m), 6.6-7.0 (6H, m), 7.2-7.3 (5H, m)

Ms: 618 (M+22)

### (2) [3-[(7S)-7-[N-[(2R)-2-(3-Chlorophenyl)-2-hydroxyethyl]-N-(tert-butoxycarbonyl)amino]-5,6,7,8-tetrahydro-2-naphthalenyl]phenoxy]acetic Acid Ethyl Ester

¹H NMR (200 MHz, CDCl₃, δ): 1.30 (3H, t, J=7.4 Hz), 1.51 (9H, s), 1.8-2.0 (2H, m), 2.8-3.1 (4H, m), 3.2-3.7 (2H, m) 4.0-4.3 (1H, m), 4.22 (2H, q, J=7.4 Hz), 4.67 (2H, s), 4.93 (1H, m), 6.8-7.0 (1H, m), 7.1-7.5 (10H, m)

Ms: 601 (M+22)

### (3) [4-[(7S)-7-[N-[(2R)-2-(3-Chlorophenyl)-2-hydroxyethyl]-N-(tert-butoxycarbonyl)amino]-5,6,7,8-tetrahydro-2-naphthalenyl]phenoxy]acetic Acid Ethyl Ester

¹H NMR (200 MHz, CDCl₃, δ): 1.30 (3H, t, J=7.4 Hz), 1.55 (9H, s), 1.8-2.0 (2H, m), 2.8-3.1 (4H, m), 3.2-3.7 (2H, m) 4.0-4.3 (1H, m), 4.22 (2H, q, J=7.4 Hz), 4.66 (2H, s), 4.92 (1H, m), 6.97 (2H, d, J=8 Hz), 7.13 (1H, d, J=8 Hz), 7.2-7.6 (8H, m)

Ms: 601 (M+22)

### EXAMPLE 7

To a mixture of (7S)-7-[N-[(2R)-2-(3-chlorophenyl)-2-hydroxyethyl]-N-(tert-butoxycarbonyl)amino]-2-hydroxy-5,6,7,8-tetrahydronaphthalene (300 mg) in dimethyl sulfoxide (10 ml) were added ethyl 6-chloronicotinate (300 mg) and potassium carbonate (800 mg) at room temperature, and the mixture was stirred at 80° C. for 2 hours. The resulting mixture was poured into a mixture of ethyl acetate and water, and the organic layer was washed with brine. After the solvent was evaporated under reduced pressure, the residue was purified by column chromatography on silica gel to give 6-[[(7S)-7-[N-[(2R)-2-(3-chlorophenyl)-2-hydroxyethyl]-N-(tert-butoxycarbonyl)amino]-5,6,7,8-tetrahydro-2-naphthalenyl]oxy]nicotinic acid ethyl ester (300 mg).

¹H NMR (200 MHz, CDCl₃, δ): 1.34 (3H, t, J=7.0 Hz), 1.52 (9H, s), 1.7-2.0 (2H, m), 2.6-3.0 (4H, m), 3.2-3.6 (2H, m), 4.35 (2H, q, J=7.0 Hz), 4.90 (1H, m), 6.8-7.2 (4H, m), 7.2-7.4 (4H, m), 8.27 (1H, dd, J=2.2, 8.4 Hz), 8.81 (1H, dd, J=2.2 Hz)

Ms: 589 (M+22)

### EXAMPLE 8

The following compounds were obtained according to a similar manner to that of Example 7.

### (1) 2-[[(7S)-7-[N-[(2R)-2-(3-Chlorophenyl)-2-hydroxyethyl]-N-(tert-butoxycarbonyl)amino]-5,6,7,8-tetrahydro-2-naphthalenyl]oxy]-3-pyridylcarboxaldehyde

¹H NMR (200 MHz, CDCl₃, δ): 1.56 (9H, s), 1.7-2.0 (2H, m), 2.7-3.0 (4H, m), 3.1-3.7 (2H, m), 4.0-4.2 (1H, m), 4.88 (1H, m), 6.8-7.2 (7H, m), 7.39 (1H, s), 8.23 (1H, dd, J=2.2, 7.2 Hz), 8.36 (1H, dd, J=2.2 Hz), 10.52 (1H, s)

Ms: 523 (M+1)

### (2) 5-[[(7S)-7-[N-[(2R)-2-(3-Chlorophenyl)-2-hydroxyethyl]-N-(tert-butoxycarbonyl)amino]-5,6,7,8-tetrahydro-2-naphthalenyl]oxy]-2-thiophenecarboxaldehyde

¹H NMR (200 MHz, CDCl₃, δ): 1.51 (9H, s), 1.7-2.0 (2H, m), 2.7-3.0 (4H, m), 3.1-3.3 (1H, m), 2.3-2.5 (1H, m), 4.0-4.3 (1H, m), 4.8-5.0 (1H, m), 6.5-6.8 (2H, m), 6.8-7.6 (7H, m), 9.70 (1H, s)

Ms: 550 (M+22)

### (3) 4-[[(8S)-8-[N-[(2R)-2-(3-Chlorophenyl)-2-hydroxyethyl]-N-(tert-butoxycarbonyl)amino]-6,7,8,9-tetrahydro-5H-benzo[a]cyclohepten-2-yl]oxy]benzoic Acid Methyl Ester

¹H NMR (200 MHz, CDCl₃, δ): 1.2-1.5 (1H, m), 1.51 (9H, s), 1.8-2.1 (2H, m), 2.5-2.8 (3H, m), 3.2-3.7 (4H, m) 4.9-5.1 (2H, m), 6.5-6.6 (2H, m), 6.8-7.1 (2H, m), 7.2-7.7 (5H, m), 9.70 (1H, s)

Ms: 564 (M+22)

### EXAMPLE 9

To a mixture of 2-[[(7S)-7-[N-[(2R)-2-(3-chlorophenyl)-2-hydroxyethyl]-N-(tert-butoxycarbonyl)amino]-5,6,7,8-tetrahydro-2-naphthalenyl]oxy]-3-pyridylcarboxaldehyde (300 mg), acetonitrile (5 ml), pH 4 buffer solution (sodium dihydrogenphosphate) (0.25 ml), and 30% hydrogen peroxide solution (0.12 ml), sodium chlorite (500 mg) was added at room temperature. The reaction mixture was stirred at the same temperature for 4 hours, diluted with ethyl acetate (50 ml), washed with water followed by brine, dried over magnesium sulfate, and evaporated to give the corresponding acid. The obtained acid was diluted with 6N hydrogen chloride in dioxane (10 ml) and the mixture was allowed to keep at room temperature for 4 hours. The mixture was evaporated under reduced pressure and the obtained solid was washed with ethyl ether to give 2-[[(7S)-7-[[(2R)-2-(3-chlorophenyl)-2-hydroxyethyl]amino]-5,6,7,8-tetrahydro-2-naphthalenyl]oxy]nicotinic acid hydrochloride (200 mg).

¹H NMR (200 MHz, DMSO-d₆, δ): 1.7-2.0 (1H, m), 2.3-2.5 (1H, m), 2.7-3.7 (7H, m), 5.12 (1H, m), 6.37 (1H, m), 6.7-7.0 (2H, m), 7.1-7.3 (2H, m), 7.4-7.7 (4H, m), 8.1-8.3 (2H, m), 8.9 (1H, m), 9.5 (1H, m),

Ms: 439 (M+1)

### EXAMPLE 10

To a mixture of 2-[[(7S)-7-[N-[(2R)-2-(3-chlorophenyl)-2-hydroxyethyl]-N-(tert-butoxycarbonyl)amino]-5,6,7,8-tetrahydro-2-naphthalenyl]oxy]-3-pyridylcarboxaldehyde (300 mg) in toluene (20 ml) was added (carbethoxymethylene)triphenylphosphorane (300 mg) at room temperature. The reaction mixture was stirred at 120° C. for 4 hours, and evaporated under reduced pressure. The residue was purified by column chromatography on silica gel to give the ester. To a solution of the ester in methanol (10 ml) was added 1N sodium hydroxide (5 ml) at room temperature, and the mixture was stirred at the same temperature for 12 hours. The resulting mixture was evaporated under reduced pressure. The residue was diluted with a mixture of ethyl acetate (30 ml) and 1N hydrochloric acid (10 ml), and the organic layer was washed with brine, dried over magnesium sulfate, and evaporated under reduced pressure. The obtained acid was diluted with 6N hydrogen chloride in dioxane (10 ml) and the mixture was allowed to keep at room temperature for 4 hours. The mixture was evaporated under reduced pressure and the obtained solid was washed with ethyl ether to give 3-[2-[[(7S)-7-[[(2R)-2-(3-chlorophenyl)-2-hydroxyethyl]amino]-5,6,7,8-tetrahydro-2-naphthalenyl]-oxy]-3-pyridyl]-2-propenoic acid hydrochloride (180 mg).

¹H NMR (200 MHz, DMSO-d₆, δ): 1.7-2.0 (1H, m), 2.3-2.5 (1H, m), 2.7-3.7 (7H, m), 5.12 (1H, m), 6.13 (1H, d, J=12.4 Hz), 6.8-7.5 (8H, m), 7.80 (1H, d, J=12.4 Hz), 8.1-8.3 (2H, m), 8.97 (1H, m), 9.40 (1H, m)

Ms: 465 (M+1)

### EXAMPLE 11

The following compound was obtained according to a similar manner to that of Example 7 and then according to a similar manner to that of Example 10.

### 3-[6-[[(7S)-7-[[(2R)-2-(3-Chlorophenyl)-2-hydroxyethyl]amino]-5,6,7,8-tetrahydro-2-naphthalenyl]oxy]-3-pyridyl]-2-propenoic acid hydrochloride

¹H NMR (200 MHz, DMSO-d₆, δ): 1.8-2.0 (1H, m), 2.2-2.5 (1H, m), 2.6-3.3 (7H, m), 5.14 (1H, m), 6.57 (1H, d, J=16.2 Hz), 6.8-7.2 (4H, m), 7.3-7.5 (4H, m), 7.58 (1H, d, J=16.2 Hz), 8.23 (1H, dd, J=2.2, 8.8 Hz), 8.40 (1H, d, J=2.2 Hz), 9.07 (1H, m), 9.7 (1H, m)

Ms: 465 (M+1)

### EXAMPLE 12

To a mixture of (7S)-7-[N-[(2R)-2-(3-chlorophenyl)-2-hydroxyethyl]-N-(tert-butoxycarbonyl)amino]-2-hydroxy-5,6,7,8-tetrahydronaphthalene (300 mg) in dimethyl sulfoxide (10 ml) were added 2-chloro-3-cyanopyridine (100 mg) and potassium carbonate (800 mg) at room temperature, and the mixture was stirred at 80° C. for 2 hours. The resulting mixture was poured into a mixture of ethyl acetate and water, and the organic layer was washed with brine. After the solvent was evaporated under reduced pressure, the residue was diluted in N,N-dimethylformamide (5 ml). To the mixture were added sodium azide (100 mg) and ammonium chloride (200 mg), and stirred at 120° C. for 12 hours. The resulting mixture was poured into a mixture of ethyl acetate and water, and the organic layer was washed with brine. After the solvent was evaporated under reduced pressure, the residue was purified by column chromatography on silica gel to give the corresponding tetrazole (190 mg). The obtained terazole was diluted with 6N hydrogen chloride in dioxane (10 ml) and the mixture was allowed to keep at room temperature for 4 hours. The mixture was evaporated under reduced pressure and the obtained solid was washed with ethyl ether to give (1R)-1-(3-chlorophenyl)-2-[[(2S)-7-[[3-(1H-tetrazol-5-yl)-2-pyridyl]oxy]-1,2,3,4-tetrahydro-2-naphthalenyl]amino]ethanol hydrochloride (150 mg).

¹H NMR (200 MHz, DMSO-d₆, δ): 1.7-2.0 (1H, m), 2.3-2.5 (1H, m), 2.7-3.7 (7H, m), 5.08 (1H, m), 6.38 (1H, m), 7.0-7.6 (8H, m), 8.29 (1H, m), 8.50 (1H, m), 8.96 (1H, m), 9.43 (1H, m)

Ms: 463 (M+1)

### EXAMPLE 13

The following compounds were obtained according to a similar manner to that of Example 9.

### (1) 5-[[(7S)-7-[[(2R)-2-(3-Chlorophenyl)-2-hydroxyethyl]-amino]-5,6,7,8-tetrahydro-2-naphthalenyl]oxy]-2-thiophenecarboxylic acid hydrochloride

¹H NMR (200 MHz, DMSO-d₆, δ): 1.8-2.2 (2H, m), 2.4-3.4 (7H, m), 5.05 (1H, m), 6.36 (1H, m), 6.5-7.5 (9H, m), 8.93 (1H, m), 9.38 (1H, m)

Ms: 444 (M+1)

### (2) 5-[[(8S)-8-[[(2R)-2-(3-Chlorophenyl)-2-hydroxyethyl]-amino]-6,7,8,9-tetrahydro-5H-benzo[a]cyclohepten-2-yl]oxy]-2-thiophenecarboxylic acid hydrochloride

¹H NMR (200 MHz, DMSO-d₆, δ): 1.2-1.5 (1H, m), 1.7-2.3 (3H, m), 2.5-3.3 (7H, m), 4.97 (1H, m), 6.33 (1H, br.s), 6.62 (1H, d, J=8.4 Hz), 7.0-7.6 (8H, m), 8.75 (1H, m), 8.99 (1H, m)

Ms: 458 (M+1)

### EXAMPLE 14

The following compounds were obtained according to a similar manner to that of Example 10.

### (1) 3-[5-[[(7S)-7-[[(2R)-2-(3-Chlorophenyl)-2-hydroxyethyl]amino]-5,6,7,8-tetrahydro-2-naphthalenyl]-oxy]-2-thienyl]-2-propenoic acid hydrochloride

¹H NMR (200 MHz, DMSO-d₆, δ): 1.7-2.0 (1H, m), 2.3-2.5 (1H, m), 2.7-3.7 (7H, m), 5.06 (1H, m), 6.3-6.7 (4H, m), 6.8-7.4 (5H, m), 8.89 (1H, m), 9.19 (1H, m)

Ms: 470 (M+1)

### (2) 3-[5-[[(8S)-8-[[(2R)-2-(3-Chlorophenyl)-2-hydroxyethyl]amino]-6,7,8,9-tetrahydro-5H-benzo[a]cyclohepten-2-yl]oxy]-2-thienyl]-2-propenoic acid hydrochloride

¹H NMR (200 MHz, DMSO-d₆, δ): 1.1-1.3 (1H, m), 1.7-2.2 (3H, m), 2.5-3.5 (7H, m), 4.96 (1H, m), 6.33 (1H, m), 6.5-7.6 (9H, m), 8.72 (1H, m), 8.95 (1H, m)

Ms: 484 (M+1)

### EXAMPLE 15

To a mixture of (7S)-7-[N-[(2R)-2-(3-chlorophenyl)-2-hydroxyethyl]-N-(tert-butoxycarbonyl)amino]-2-hydroxy-5,6,7,8-tetrahydronaphthalene (400 mg) in dichloromethane (10 ml) were added 2,6-lutidine (0.22 ml) and trifluoromethanesulfonic anhydride (0.16 ml) at −78° C. under nitrogen, then stirred for 1 hour at the same temperature. The mixture was poured into water and the organic layer was washed with 1N-hydrochloric acid and brine respectively, then dried over magnesium sulfate. After filtration, the solvent was evaporated, and the obtained residue was purified by column chromatography on silica gel to give the corresponding sulfonate. To a solution of the sulfonate in diethoxymethane (10 ml) were added (3-methoxycarbonylphenyl)boronic acid (200 mg) and tetrakis(triphenylphosphine)palladium(0) (110 mg) and 2N sodium carbonate (2 mg) at room temperature, and the mixture was stirred at 80° C. for 2 hours. The resulting mixture was filtrated by celite and the mother layer was evaporated under reduced pressure. The residue was purified by column chromatography on silica gel to give 3-[(7S)-7-[N-[(2R)-2-(3-chlorophenyl)-2-hydroxyethyl]-N-(tert-butoxycarbonyl)amino]-5,6,7,8-tetrahydro-2-naphthalenyl]benzoic acid methyl ester (350 mg).

¹H NMR (200 MHz, CDCl₃, δ): 1.52 (9H, s), 1.8-2.0 (2H, m), 2.8-3.1 (4H, m), 3.2-3.7 (2H, m), 3.95 (3H, s), 4.0-4.3 (1H, m), 4.93 (1H, m), 7.0-7.5 (8H, m), 7.78 (1H, d, J=8 Hz), 7.99 (1H, d, J=8 Hz), 8.26 (1H, s)

Ms: 558 (M+22)

### EXAMPLE 16

The following compounds were obtained according to a similar manner to that of Example 15.

### (1) 4-[(7S)-7-[N-[(2R)-2-(3-Chlorophenyl)-2-hydroxyethyl]-N-(tert-butoxycarbonyl)amino]-5,6,7,8-tetrahydro-2-naphthalenyl]benzoic acid methyl ester

¹H NMR (200 MHz, CDCl₃, δ): 1.52 (9H, s), 1.8-2.0 (2H, m), 2.8-3.1 (4H, m), 3.2-3.7 (2H, m), 3.94 (3H, s), 4.0-4.3 (1H, m), 4.93 (1H, m), 7.1-7.4 (8H, m), 7.64 (2H, d, J=8.4 Hz), 8.09 (2H, d, J=8.4 Hz), 8.48 (1H, s)

Ms: 558 (M+22)

### (2) [3-[(7S)-7-[N-[(2R)-2-(3-Chlorophenyl)-2-hydroxyethyl]-N-(tert-butoxycarbonyl)amino]-5,6,7,8-tetrahydro-2-naphthalenyl]phenoxy](tert-butyl)dimethylsilane

¹H NMR (200 MHz, CDCl₃, δ): 0.19 (6H, s), 0.96 (9H, s), 1.54 (9H, s), 1.8-2.0 (2H, m), 2.8-3.1 (4H, m), 3.2-3.7 (2H, m), 4.0-4.3 (1H, m), 4.9 (1H, m), 6.8-7.0 (1H, m), 7.0-7.4 (10H, m)

Ms: 630 (M+22)

### (3) [4-[(7S)-7-[N-[(2R)-2-(3-Chlorophenyl)-2-hydroxyethyl]-N-(tert-butoxycarbonyl)amino]-5,6,7,8-tetrahydro-2-naphthalenyl]phenoxy](tert-butyl)dimethylsilane

¹H NMR (200 MHz, CDCl₃, δ): 0.21 (6H, s), 1.01 (9H, s), 1.57 (9H, s), 1.8-2.0 (2H, m), 2.8-3.1 (4H, m), 3.2-3.7 (2H, m), 4.0-4.3 (1H, m), 4.9 (1H, m), 6.89 (2H, d, J=8 Hz), 7.12 (1H, d, J=8 Hz), 7.2-7.5 (8H, m)

Ms: 630 (M+22)

Preparation 3

The following compound was obtained according to a similar manner to that of Preparation 8.

### (7S)-7-[[(Benzyloxy)carbonyl]amino]-5,6,7,8-tetrahydro-2-naphthalenyl Trifluoromethanesulfonate

Ms (m/z): 430 (M+1)

Preparation 4

To a solution of (7S)-7-[[(benzyloxy)carbonyl]amino]-5,6,7,8-tetrahydro-2-naphthalenyl trifluoromethanesulfonate (750 mg) in 1,2-dimethoxyethane (15 ml) was added 4-(methoxycarbonyl)phenylboronic acid (440 mg), tetrakis(triphenylphosphine)palladium (101 mg) and aqueous solution of sodium carbonate (2M, 7 ml), and the mixture was stirred at 75° C. for 10 hours under nitrogen. The mixture was diluted with ethyl acetate and water. The organic layer was separated, washed with brine, dried over magnesium sulfate and evaporated. The residue was purified by column chromatography on silica gel (hexane/ethyl acetate=2/1) to give methyl 4-[(7S)-7-[[(benzyloxy)carbonyl]amino]-5,6,7,8-tetrahydro-2-naphthalenyl]benzoate (580 mg) as a colorless powder.

Ms (m/z): 416 (M+1)

Preparation 5

The following compounds were obtained according to a similar manner to that of Example 25 starting from the object compound of Preparation 4 or 3.

### (1) Methyl 4-[(7S)-7-amino-5,6,7,8-tetrahydro-2-naphthalenyl]benzoate

Ms (m/z): 282 (M+1)

### (2) (7S)-7-Amino-5,6,7,8-tetrahydro-2-naphthalenol

Ms (m/z): 164 (M+1)

### (3) Ethyl 6-[(7S)-7-amino-5,6,7,8-tetrahydro-2-naphthalenyl]nicotinate

(+)ESI-Ms (m/z): 297 (M+1)⁺

Preparation 6

The following compound was obtained according to a similar manner to that of Example 17.

### (7S)-7-[[(2R)-2-(4-Chlorophenyl)-2-hydroxyethyl]amino]-5,6,7,8-tetrahydro-2-naphthalenol

Ms (m/z): 318 (M+1)

Preparation 7

To a solution of ethyl(7S)-7-[[(2R)-2-(4-chlorophenyl)-2-hydroxyethyl]amino]-5,6,7,8-tetrahydro-2-naphthalenol (9.75 g) in tetrahydrofuran (100 ml) was added di-tert-butyl dicarbonate (6.7 g), and the mixture was stirred at room temperature for 2 hours. The mixture was evaporated. The residue was purified by column chromatography on silica gel (hexane/ethyl acetate=2/1) to give tert-butyl [(2R)-2-(4-chlorophenyl)-2-hydroxyethyl][(2S)-7-hydroxy-1,2,3,4-tetrahydro-2-naphthalenyl]carbamate (12.22 g) as a colorless foam.

Ms (m/z): 418 (M+1)

Preparation 8

Under nitrogen at −60° C., to a solution of tert-butyl [(2R)-2-(4-chlorophenyl)-2-hydroxyethyl][(2S)-7-hydroxy-1,2,3,4-tetrahydro-2-naphthalenyl]carbamate (6.04 g) and 2,6-lutidine (3.37 ml) in dichloromethane (100 ml) was added trifluoromethanesulfonic anhydride (2.43 ml), and the mixture was stirred at the same temperature for 1 hour. The resulting mixture was poured into aqueous ammonia and the aqueous mixture was extracted with ethyl acetate. The organic layer was washed successively with 1N hydrochloric acid, water, saturated aqueous sodium bicarbonate and brine, dried over anhydrous magnesium sulfate and evaporated under reduced pressure. The residue was purified by column chromatography on silica gel (hexane/ethyl acetate=1/1) to give (7S)-7-[-N-(tert-butoxycarbonyl)-N-[(2R)-2-(4-chlorophenyl)-2-hydroxyethyl]amino]-5,6,7,8-tetrahydro-2-naphthalenyl trifluoromethanesulfonate (6.56 g) as a colorless foam.

Ms (m/z): 550 (M+1)

Preparation 9

To a solution of AD mix-beta (10.1 g) (cf. JOC vol. 57, No. 10, 1992, 2768-2771) in a mixture of tert-butanol (60 ml) and water (60 ml) was added 1-chloro-4-vinylbenzene (1.0 g) on ice-cooling and the mixture was stirred at the same temperature for 4 hours. To the mixture was added sodium sulfite (19 g). The resulting mixture was poured into saturated aqueous sodium bicarbonate solution, and extracted with ethyl acetate. The organic layer was washed with brine, dried over magnesium sulfate, and evaporated in vacuo to give (1R)-1-(4-chlorophenyl)-1,2-ethanediol (1.04 g) as a colorless oil.

NMR (CDCl₃, δ): 3.50-3.80 (2H, m), 4.70-4.85 (1H, m), 7.20-7.40 (4H, m)

Preparation 10

Trimethylsilyl chloride (0.956 ml) was added to the solution of (1R)-1-(4-chlorophenyl)-1,2-ethanediol (1.0 g) and trimethyl orthoacetate (0.87 ml) in dichloromethane (30 ml) on ice-cooling. The solution was stirred for 1 hour and evaporated. The crude product was dissolved in dry methanol and potassium carbonate (1.97 g) was added. The suspension was stirred vigorously for 100 minutes, then filtered and the residue was washed with dichloromethane. The filtrate was washed with brine, dried over magnesium sulfate, and evaporated in vacuo to give (2R)-2-(4-chlorophenyl)oxirane (700 mg) as a colorless oil.

NMR (CDCl₃, δ): 2.75 (1H, dd, J=2.5, 5.5 Hz), 3.14 (1H, dd, J=4.0, 5.5 Hz), 3.80-3.86 (1H, m), 7.18-7.40 (4H, m)

Preparation 11

To a solution of methyl 4-bromo-2-methoxybenzoate (2.0 g) in 1,4-dioxane (40 ml) was added bis(pinacolato)diboron (2.07 g), dichlorobis(triphenylphosphine)palladium(II) (286 mg) and potassium acetate (2.4 g), and the mixture was stirred at 95° C. for 10 hours under nitrogen. The mixture was diluted with ethyl acetate and water. The organic layer was separated, washed with brine, dried over magnesium sulfate and evaporated. The residue was purified by column chromatography on silica gel (hexane/ethyl acetate=3/1) to give methyl 2-methoxy-4-(4,4,5,5-tetramethyl-1,3,2-dioxaborolan-2-yl)benzoate (2.0 g).

Ms (m/z): 293 (M+1)

Preparation 12

To a suspension of methyl 2-methoxy-4-(4,4,5,5-tetramethyl-1,3,2-dioxaborolan-2-yl)benzoate (2.0 g) in a mixture of acetone (70 ml) and water (70 ml) were added ammonium acetate (1.11 g) and sodium periodate (3.08 g), and the mixture was stirred at room temperature for 15 hours. The solvent was evaporated and the residue was diluted with ethyl acetate. The organic layer was separated, washed with water and brine, dried over magnesium sulfate and evaporated under reduced pressure to give [3-methoxy-4-(methoxycarbonyl)phenyl]boronic acid (1.4 g) as a colorless powder.

Ms (m/z): 209 (M−1)

### EXAMPLE 17

A solution of methyl 4-[(7S)-7-amino-5,6,7,8-tetrahydro-2-naphthalenyl]benzoate (142 mg), and (2R)-2-(4-chlorophenyl)oxirane (70.2 mg) in ethanol (10 ml) was refluxed for 18 hours. The mixture was evaporated in vacuo. The residue was purified by column chromatography on silica gel (chloroform/methanol=100/1) to give methyl 4-[(7S)-7-[[(2R)-2-(4-chlorophenyl)-2-hydroxyethyl]amino]-5,6,7,8-tetrahydro-2-naphthalenyl]benzoate (130 mg) as a colorless foam.

Ms (m/z): 436 (M+1)

### EXAMPLE 18

The following compound was obtained according to a similar manner to that of Example 17.

### Methyl 4-[(7S)-7-[[(2R)-2-(6-chloro-3-pyridyl)-2-hydroxyethyl]amino]-5,6,7,8-tetrahydro-2-naphthalenyl]-benzoate

Ms (m/z): 437 (M+1)

### EXAMPLE 19

To a solution of methyl 4-[(7S)-7-[[(2R)-2-(4-chlorophenyl)-2-hydroxyethyl]amino]-5,6,7,8-tetrahydro-2-naphthalenyl]benzoate (130 mg) in methanol (5.0 ml) was added 1N sodium hydroxide (0.688 ml) and the mixture was stirred for 2 hours at room temperature. The mixture was evaporated in vacuo to give sodium 4-[(7S)-7-[[(2R)-2-(4-chlorophenyl)-2-hydroxyethyl]amino]-5,6,7,8-tetrahydro-2-naphthalenyl]benzoate (120 mg) as a colorless powder.

NMR (DMSO-d₆, δ): 1.40-1.60 (1H, m), 1.90-2.10 (1H, m), 2.50-3.20 (6H, m), 4.60-4.70 (1H, m), 7.05 (1H, d, J=8 Hz), 7.30-7.40 (6H, m), 7.50 (2H, d, J=8 Hz), 7.90 (2H, d, J=8 Hz)

Ms (m/z): 422 (M+1)

### EXAMPLE 20

The following compound was obtained according to a similar manner to that of Preparation 4.

### Methyl 4-[(7S)-7-[N-(tert-butoxycarbonyl)-N-[(2R)-2-(4-chlorophenyl)-2-hydroxyethyl]amino]-5,6,7,8-tetrahydro-2-naphthalenyl]-2-methoxybenzoate

Ms (m/z): 566 (M+1)

### EXAMPLE 21

The following compound was obtained according to a similar manner to that of Example 26.

### 4-[(7S)-7-[[(2R)-2-(4-Chlorophenyl)-2-hydroxyethyl]-amino]-5,6,7,8-tetrahydro-2-naphthalenyl]-2-methoxybenzoic acid hydrochloride

NMR (DMSO-d₆, δ): 1.80-1.90 (1H, m), 2.30-2.40 (1H, m), 2.80-3.20 (6H, m), 3.90 (3H, s), 5.00-5.05 (1H, m), 7.10-7.30 (3H, m), 7.50-7.60 (6H, m), 7.70 (2H, d, J=8 Hz)

Ms (m/z): 452 (M+1)

### EXAMPLE 22

The following compound was obtained according to a similar manner to that of Preparation 7.

### Methyl 4-[(7S)-7-[N-(tert-butoxycarbonyl)-N-[(2R)-2-(6-chloro-3-pyridyl)-2-hydroxyethyl]amino]-5,6,7,8-tetrahydro-2-naphthalenyl]benzoate

Ms (m/z): 537 (M+1)

### EXAMPLE 23

To a solution of methyl 4-[(7S)-7-[N-(tert-butoxycarbonyl)-N-[(2R)-2-(6-chloro-3-pyridyl)-2-hydroxyethyl]amino]-5,6,7,8-tetrahydro-2-naphthalenyl]-benzoate (1.0 g) in ethanol (15.0 ml) was added 1N sodium hydroxide (5.0 ml) and the mixture was stirred for 2 hours at room temperature. The mixture was diluted with ethyl acetate and 1N hydrochloric acid. The organic layer was separated, washed with brine, dried over magnesium sulfate and evaporated. The residue was purified by column chromatography on silica gel (hexane/ethyl acetate=1/1) to give 4-[(7S)-7-[N-(tert-butoxycarbonyl)-N-[(2R)-2-hydroxy-2-(6-chloro-3-pyridyl)ethyl]amino]-5,6,7,8-tetrahydro-2-naphthalenyl]benzoic acid (800 mg) as a colorless foam.

Ms (m/z): 523 (M+1)

### EXAMPLE 24

The following compound was obtained according to a similar manner to that of Example 23.

### 4-[(7S)-7-[N-(tert-Butoxycarbonyl)-N-[(2R)-2-(4-chlorophenyl)-2-hydroxyethyl]amino]-5,6,7,8-tetrahydro-2-naphthalenyl]-2-methoxybenzoic Acid

Ms (m/z): 552 (M+1)

### EXAMPLE 25

4-[(7S)-7-[N-(tert-Butoxycarbonyl)-N-[(2R)-2-hydroxy-2-(6-chloro-3-pyridyl)ethyl]amino]-5,6,7,8-tetrahydro-2-naphthalenyl]benzoic acid (800 mg), ammonium formate (300 mg) and palladium on carbon powder (100 mg) in a mixture of methanol (25 ml) and water (5.0 ml) was refluxed for 15 minutes. The reaction mixture was filtrated and poured into water and extracted with ethyl acetate. The organic layer was washed with brine, dried over magnesium sulfate, and evaporated in vacuo. A mixture of the residue was purified by column chromatography on silica gel (chloroform/methanol=99/1) to give 4-[(7S)-7-[N-(tert-butoxycarbonyl)-N-[(2R)-2-hydroxy-2-(3-pyridyl)ethyl]amino]-5,6,7,8-tetrahydro-2-naphthalenyl]benzoic acid (620 mg) as a colorless foam.

Ms (m/z): 489 (M+1)

### EXAMPLE 26

A solution of 4-[(7S)-7-[N-(tert-butoxycarbonyl)-N-[(2R)-2-hydroxy-2-(3-pyridyl)ethyl]amino]-5,6,7,8-tetrahydro-2-naphthalenyl]benzoic acid (620 mg) and 4N hydrogen chloride in dioxane (10 ml) was stirred at room temperature for 24 hours. The resultant solid was collected by filtration and dried to give 4-[(7S)-7-[[(2R)-2-hydroxy-2-(3-pyridyl)ethyl]amino]-5,6,7,8-tetrahydro-2-naphthalenyl]benzoic acid dihydrochloride (450 mg) as a white solid.

NMR (DMSO-d₆, δ): 1.80-1.90 (1H, m), 2.30-2.40 (1H, m), 2.80-3.50 (6H, m), 5.30-5.40 (1H, m), 7.20 (1H, d, J=8 Hz), 7.40-7.50 (2H, m), 7.77 (2H, d, J=8 Hz), 7.90-8.05 (3H, m), 8.60 (1H, d, J=8 Hz), 8.88 (1H, d, J=8 Hz), 8.99 (1H, s)

Preparation 13

To a solution of 4-bromo-2-fluorobenzoate (1.5 g) in N,N-dimethylformamide (30 ml) was added bis(pinacolate)-diboron (1.8 g), 1,1′-bis(diphenylphosphino)-ferrocenedichlorobispalladium(II), complex with dichloromethane (263 mg) and potassium acetate (1.9 g), and the mixture was stirred at 100° C. for 18 hours under nitrogen. The mixture was diluted with ethyl acetate and water. The organic layer was separated, washed with brine, dried over magnesium sulfate and evaporated. The residue was purified by column chromatography on silica gel (hexane/ethyl acetate=5/1) to give methyl 2-fluoro-4-(4,4,5,5-tetramethyl-1,3,2-dioxaborolan-2-yl)benzoate (350 mg).

(+)ESI-MS (m/z): 303 (M+Na)⁺

Preparation 14

The following compound was obtained according to a similar manner to that of Preparation 13.

### Benzyl(2S)-7-(4,4,5,5-tetramethyl-1,3,2-dioxaborolan-2-yl)-1,2,3,4-tetrahydro-2-naphthalenylcarbamate

(+)ESI-MS (m/z): 430 (M+Na)⁺

Preparation 15

To a solution of (7S)-7-[[(benzyloxy)carbonyl]amino]-5,6,7,8-tetrahydro-2-naphthalenyl trifluoromethanesulfonate (750 mg) in 1,2-dimethoxyethane (15 ml) was added 4-(methoxycarbonyl)phenylboronic acid (440 mg), tetrakis(triphenylphosphine)palladium (101 mg) and aqueous solution of sodium carbonate (2M, 7 ml), and the mixture was stirred at 75° C. for 10 hours under nitrogen. The mixture was diluted with ethyl acetate and water. The organic layer was separated, washed with brine, dried over magnesium sulfate and evaporated. The residue was purified by column chromatography on silica gel (hexane/ethyl acetate=2/1) to give methyl 4-[(7S)-7-[[(benzyloxy)carbonyl]amino]-5,6,7,8-tetrahydro-2-naphthalenyl]benzoate (580 mg) as a colorless powder.

MS (m/z): 416 (M+1)

Preparation 16

The following compounds were obtained according to a similar manner to that of Preparation 15.

### (1) Ethyl 4-[(7S)-7-[[(benzyloxy)carbonyl]amino]-5,6,7,8-tetrahydro-2-naphthalenyl]-3-methoxybenzoate

MS (m/z): 460 (M+1)

### (2) Ethyl 6-[(7S)-7-[[(benzyloxy)carbonyl]amino]-5,6,7,8-tetrahydro-2-naphthalenyl]nicotinate

(+)ESI-MS (m/z): 453 (M+Na)+

Preparation 17

A solution of methyl 4-[(7S)-7-[[(benzyloxy)carbonyl]-amino]-5,6,7,8-tetrahydro-2-naphthalenyl]benzoate (580 mg), ammonium formate (300 mg) and palladium on carbon powder (100 mg) in methanol (25 ml) and water (5.0 ml) was refluxed for 15 minutes. The reaction mixture was filtrated and poured into water and extracted with ethyl acetate. The organic layer was washed with brine, dried over magnesium sulfate, and evaporated in vacuo. A mixture of the residue was chromatographed (chloroform-methanol) over silica gel to give methyl 4-[(7S)-7-amino-5,6,7,8-tetrahydro-2-naphthalenyl]benzoate (450 mg) as a colorless foam.

MS (m/z): 282 (M+1)

Preparation 18

The following compounds were obtained according to a similar manner to that of Preparation 17.

### (1) (7S)-7-Amino-5,6,7,8-tetrahydro-2-naphthalenol

MS (m/z): 164 (M+1)

### (2) Ethyl 4-[(7S)-7-amino-5,6,7,8-tetrahydro-2-naphthalenyl]-3-methoxybenzoate

MS (m/z): 326 (M+1)

### (3) Ethyl 1-[(7S)-7-amino-5,6,7,8-tetrahydro-2-naphthalenyl]-4-piperidinecarboxylate

MS (m/z): 303 (M+1)

### (4) Methyl 5-[[(7S)-7-amino-5,6,7,8-tetrahydro-2-naphthalenyl]oxy]-2-(1-pyrrolidinyl)benzoate

(+)ESI-MS (m/z): 367 (M+1)+

Preparation 19

A solution of (7S)-7-amino-5,6,7,8-tetrahydro-2-naphthalenol (11.2 g) and (2R)-2-(4-chlorophenyl)oxirane (9.02 g) in ethanol (10 ml) was refluxed for 18 hours. The mixture was evaporated in vacuo. The residue was purified by column chromatography on silica gel (chloroform:methanol 100:1) to give (7S)-7-[[(2R)-2-(4-chlorophenyl)-2-hydroxyethyl]amino]-5,6,7,8-tetrahydro-2-naphthalenol (9.74 g) as a colorless foam.

MS (m/z): 318 (M+1)

Preparation 20

The following compounds were obtained according to a similar manner to that of Preparation 19.

### (1) (7S)-7-[[(2R)-2-(6-Chloro-3-pyridyl)-2-hydroxyethyl]-amino]-5,6,7,8-tetrahydro-2-naphthalenol

MS (m/z): 319 (M+1)

### (2) (7S)-7-[N-Benzyl-N-[(2R)-2-(6-chloro-3-pyridyl)-2-hydroxyethyl]amino]-5,6,7,8-tetrahydro-2-naphthalenol

MS (m/z): 409 (M+1)

### (3) (7S)-7-[[(2R)-2-Hydroxy-2-(4-methylphenyl)ethyl]amino]-5,6,7,8-tetrahydro-2-naphthalenol

MS (m/z): 298 (M+1)

### (4) (7S)-7-[[(2R)-2-(5,6-Dichloro-3-pyridyl)-2-hydroxyethyl]amino]-5,6,7,8-tetrahydro-2-naphthalenol

MS (m/z): 353 (M+1)

Preparation 21

To a solution of ethyl(7S)-7-[[(2R)-2-(4-chlorophenyl)-2-hydroxyethyl]amino-5,6,7,8-tetrahydro-2-naphthalenol (9.75 g) in tetrahydrofuran (100 ml) was added di-tert-butyl dicarbonate (6.7 g), and the mixture was stirred at room temperature for 2 hours. The mixture was evaporated. The residue was purified by column chromatography on silica gel (hexane/ethyl acetate=2/1) to give tert-butyl N-[(2R)-2-(4-chlorophenyl)-2-hydroxyethyl]-N-[(2S)-7-hydroxy-1,2,3,4-tetrahydro-2-naphthalenyl]-carbamate (12.22 g) as a colorless foam.

MS (m/z): 418 (M+1)

Preparation 22

The following compounds were obtained according to a similar manner to that of Preparation 21.

### (1) tert-Butyl N-[(2R)-2-(6-chloro-3-pyridyl)-2-hydroxyethyl]-N-[(2S)-7-hydroxy-1,2,3,4-tetrahydro-2-naphthalenyl]carbamate

MS (m/z): 419 (M+1)

### (2) tert-Butyl N-[(2R)-2-hydroxy-2-(4-methylphenyl)ethyl]-N-[(2S)-7-hydroxy-1,2,3,4-tetrahydro-2-naphthalenyl]-carbamate

MS (m/z): 398 (M+1)

### (3) tert-Butyl N-[(2R)-2-(5,6-dichloro-3-pyridyl)-2-hydroxyethyl]-N-[(2S)-7-hydroxy-1,2,3,4-tetrahydro-2-naphthalenyl]carbamate

MS (m/z): 475 (M+Na)

Preparation 23

Under nitrogen at −60° C., to a solution of tert-butyl N-[(2R)-2-(4-chlorophenyl)-2-hydroxyethyl]-N-[(2S)-7-hydroxy-1,2,3,4-tetrahydro-2-naphthalenyl]carbamate (6.04 g) and 2,6-lutidine (3.37 ml) in dichloromethane (100 ml) was added trifluoromethanesulfonic anhydride (2.43 ml), and the mixture was stirred at the same temperature for 1 hour. The mixture was diluted with ethyl acetate and water. The organic layer was separated and washed successively with 1N hydrochloric acid, water, saturated aqueous sodium bicarbonate and brine, dried over anhydrous magnesium sulfate and evaporated under reduced pressure. The residue was purified by column chromatography on silica gel (hexane/ethyl acetate=1:1) to give (7S)-7-[N-(tert-butoxycarbonyl)-N-[(2R)-2-(4-chlorophenyl)-2-hydroxyethyl]amino]-5,6,7,8-tetrahydro-2-naphthalenyl trifluoromethanesulfonate (6.56 g) as a colorless foam.

MS (m/z): 550 (M+1)

Preparation 24

The following compounds were obtained according to a similar manner to that of Preparation 23.

### (1) (7S)-7-[[(Benzyloxy)carbonyl]amino]-5,6,7,8-tetrahydro-2-naphthalenyl Trifluoromethanesulfonate

MS (m/z): 430 (M+1)

### (2) (7S)-7-[N-(tert-Butoxycarbonyl)-N-[(2R)-2-(5,6-dichloro-3-pyridyl)-2-hydroxyethyl]amino]-5,6,7,8-tetrahydro-2-naphthalenyl Trifluoromethanesulfonate

MS (m/z): 585 (M+1)

### (3) (7S)-7-[N-(tert-Butoxycarbonyl)-N-[(2R)-2-hydroxy-2-(4-methylphenyl)ethyl]amino]-5,6,7,8-tetrahydro-2-naphthalenyl Trifluoromethanesulfonate

MS (m/z): 530 (M+1)

### (4) (7S)-7-[N-(tert-Butoxycarbonyl)-N-[(2R)-2-(6-chloro-3-pyridyl)-2-hydroxyethyl]amino]-5,6,7,8-tetrahydro-2-naphthalenyl Trifluoromethanesulfonate

MS (m/z): 573 (M+Na)

### (5) (7S)-7-[N-Benzyl-N-[(2R)-2-hydroxy-2-(6-methyl-3-pyridyl)ethyl]amino]-5,6,7,8-tetrahydro-2-naphthalenyl Trifluoromethanesulfonate

MS (m/z): 521 (M+1)

Preparation 25

To a solution of AD-mix-beta (10.1 g) (cf. J. Org. Chem. vol. 57, No. 10, 1992, 2768-2771) in tert-butanol (60 ml) and water (60 ml) was added 1-chloro-4-vinylbenzene (1.0 g) on ice-cooling and the mixture was stirred at the same temperature for 4 hours. To the mixture was added sodium sulfite (19 g). The resulting mixture was poured into saturated aqueous sodium bicarbonate solution, and extracted with ethyl acetate. The organic layer was washed with brine, dried over magnesium sulfate, and evaporated in vacuo to give (1R)-1-(4-chlorophenyl)-1,2-ethanediol (1.04 g) as a colorless oil.

NMR (CHCl₃, δ): 3.50-3.80 (2H, m), 4.70-4.85 (1H, m), 7.20-7.40 (4H, m)

Preparation 26

The following compound was obtained according to a similar manner to that of Preparation 25.

### (1R)-1-(4-Methylphenyl)-1,2-ethanediol

NMR (CDCl₃, δ): 3.50-3.80 (2H, m), 4.70-4.80 (1H, m), 7.10-7.30 (4H, m)

Preparation 27

Trimethylsilyl chloride (0.956 ml) was added to a solution of (1R)-1-(4-chlorophenyl)-1,2-ethanediol (1.0 g) and trimethyl orthoacetate (0.87 ml) in dichloromethane (30 ml) on ice-cooling. The solution was stirred for 1 hour and evaporated. The crude product was dissolved in dry methanol and potassium carbonate (1.97 g) was added. The suspension was stirred vigorously for 100 minutes, then filtered and the residue was washed with dichloromethane. The filtrate was washed with brine, dried over magnesium sulfate, and evaporated in vacuo to give (2R)-2-(4-chlorophenyl)oxirane (700 mg) as a colorless oil.

NMR (CHCl₃, δ): 2.75 (1H, dd, J=2.5, 5.5 Hz), 3.14 (1H, dd, J=4.0, 5.5 Hz), 3.80-3.86 (1H, m), 7.18-7.40 (4H, m)

Preparation 28

The following compound was obtained according to a similar manner to that of Preparation 27.

### (2R)-2-(4-Methylphenyl)oxirane

NMR (CDCl₃, δ): 2.34 (3H, s), 2.80 (1H, dd, J=2.5, 5.5 Hz), 3.13 (1H, dd, J=4, 5.5 Hz), 3.82 (1H, dd, J=2.5, 4 Hz), 7.10-7.30 (4H, m)

Preparation 29

Under nitrogen at room temperature, to a solution of (7S)-7-amino-5,6,7,8-tetrahydro-2-naphthalenol (3.0 g) in dichloromethane (30 ml) was added benzaldehyde (1.95 g), and the mixture was stirred at the same temperature for 20 minutes. To the mixture was added toluent and evaporated under reduced pressure. Under nitrogen, to a solution of the residue in tetrahydrofuran (20 ml) was added sodium borohydride (1.04 g) followed by methanol (10 ml) dropwise at 5° C. and the mixture was stirred at room temperature for 40 minutes. The resulting mixture was poured into a mixture of ethyl acetate and water, and stirred for 10 minutes. After separation, the organic layer was washed with brine, dried over anhydrous magnesium sulfate and evaporated under reduced pressure. The residue was purified by column chromatography on silica gel (chloroform:methanol=100:1 to 20:1) to give (7S)-7-(benzylamino)-5,6,7,8-tetrahydro-2-naphthalenol (4.0 g).

MS (m/z): 254 (M+1)

Preparation 30

Under nitrogen, to a solution of (7S)-7-[N-benzyl-N-[(2R)-2-(6-chloro-3-pyridyl)-2-hydroxyethyl]amino]-5,6,7,8-tetrahydro-2-naphthalenol (1.3 g) in tetrahydrofuran (10 ml) was added 1M methylzinc chloride in tetrahydrofuran (19 ml) and tetrakis(triphenylphosphine)palladium (147 mg) at room temperature. The mixture was stirred at 80° C. for 24 hours, and then poured into an aqueous solution (60 ml) of ethylenediaminetetraacetic acid (11 g). The resulting mixture was nutralized with saturated aqueous sodium bicarbonate and extracted with ethyl acetate. The organic layer was washed with brine, dried over anhydrous magnesium sulfate and evaporated under reduced pressure. The residue was purified by column chromatography on silica gel (chloroform:methanol=100:1) to give (7S)-7-[N-benzyl-N-[(2R)-2-hydroxy-2-(6-methyl-3-pyridyl)ethyl]amino]-5,6,7,8-tetrahydro-2-naphthalenol (1.26 g).

MS (m/z): 389 (M+1)

Preparation 31

The following compound was obtained according to a similar manner to that of Preparation 30.

### Tert-Butyl N-[(2R)-2-hydroxy-2-(6-methyl-3-pyridyl)ethyl]-N-[(2S)-7-hydroxy-1,2,3,4-tetrahydro-2-naphthalenyl]carbamate

MS (m/z): 399 (M+1)

Preparation 32

To a mixture of 1-(5,6-dichloro-3-pyridyl)ethanone (8.5 g), 1M hydrogen chloride in acetic acid (50 ml) and acetic acid (50 ml) was added N-chlorosuccinimide (7.66 g) on ice-cooling, and the mixture was stirred at room temperature for 18 hours. The resulting mixture was evaporated and poured into a mixture of water and ethyl acetate, and then stirred for 10 minutes. After separation, the organic layer was washed with brine, dried over anhydrous magnesium sulfate and evaporated under reduced pressure. The residue was purified by column chromatography on silica gel (hexane:ethyl acetate=5:1) to give 2-chloro-1-(5,6-dichloro-3-pyridyl)ethanone (6.3 g).

NMR (DMSO-d₆, δ): 4.60 (2H, s), 8.30 (1H, d, J=2 Hz), 8.80 (1H, d, J=2 Hz)

Preparation 33

To a solution of 2-chloro-1-(5,6-dichloro-3-pyridyl)ethanone (6.33 g) in tetrahydrofuran (30 ml) was added 1M (−)-B-chlorodiisopinocampheylborane in tetrahydrofuran (120 ml) on ice-cooling, and the mixture was stirred at the same temperature for 18 hours. The resulting mixture was poured into a mixture of water and ethyl acetate on ice-cooling and stirred for 10 minutes. After separation, the organic layer was washed with brine, dried over anhydrous magnesium sulfate and evaporated under reduced pressure. The residue was purified by column chromatography on silica gel (hexane:ethyl acetate=5:1) to give (1R)-2-chloro-1-(5,6-dichloro-3-pyridyl)ethanol (7.47 g).

NMR (CDCl₃, δ): 2.80 (1H, d, J=3 Hz), 3.50-3.81 (2H, m), 4.90-5.00 (1H, m), 7.88 (1H, d, J=2 Hz), 8.30 (1H, d, J=2 Hz)

Preparation 34

A solution of (1R)-2-chloro-1-(5,6-dichloro-3-pyridyl)ethanol (7.47 g) in 1N sodium hydroxide (75 ml), water (75 ml) and diethyl ether (75 ml) was stirred at room temperature for 1 hour. The resulting mixture was poured into saturated aqueous sodium bicarbonate solution, and extracted with ethyl acetate. The organic layer was washed with brine, dried over magnesium sulfate, and evaporated in vacuo to give 2,3-dichloro-5-[(2R)-2-oxiranyl]pyridine (5.88 g) as a colorless oil.

NMR (CDCl₃, δ): 2.80 (1H, dd, J=2, 5 Hz), 3.22 (1H, dd, J=4, 5 Hz), 3.80-3.90 (1H, m), 7.62 (1H, d, J=2 Hz), 8.27 (1H, d, J=2 Hz)

Preparation 35

To a solution of (7S)-7-[[(benzyloxy)carbonyl]amino]-5,6,7,8-tetrahydro-2-naphthalenyl trifluoromethanesulfonate (1.95 g) in toluene (20 ml) were added ethyl 4-piperidinecarboxylate (857 mg), palladium acetate (102 mg) and sodium tert-butoxide (611 mg), and the mixture was stirred at 70° C. for 2 hours under nitrogen. The mixture was diluted with ethyl acetate and water. The organic layer was separated, washed with brine, dried over magnesium sulfate and evaporated. The residue was purified by column chromatography on silica gel (hexane/ethyl acetate=2/1) to give ethyl 1-[(7S)-7-[[(benzyloxy)carbonyl]amino]-5,6,7,8-tetrahydro-2-naphthalenyl]-4-piperidinecarboxylate (950 mg) as a colorless powder.

MS (m/z): 437 (M+1)

Preparation 36

To a solution 2,5-dichloroisonicotinic acid (3.0 g) and potassium carbonate (2.16 g) in N,N-dimethylformamide (30 ml) was added iodoethane (1.26 ml), and the mixture was stirred at room temperature for 16 hours. The mixture was partitioned between ethyl acetate and water. The organic layer was separated, washed with water and brine, dried over magnesium sulfate and evaporated under reduced pressure to give ethyl 2,5-dichloroisonicotinate (2.76 g).

(+)ESI-MS (m/z): 242, 244 (M+Na)⁺

Preparation 37

To a solution of ethyl 3-methoxy-4-[[(trifluoromethyl)-sulfonyl]oxy]benzoate (1.52 g) in 1,4-dioxane (35 ml) were added bis(pinacolato)diboron (1.18 g), [1,1′-bis(diphenylphosphino)ferrocene]palladium(II) chloride-dichloromethane complex (309 mg) and potassium acetate (1.36 g), and the mixture was stirred at 100° C. for 10 hours under nitrogen. The mixture was diluted with ethyl acetate and water. The organic layer was separated, washed with brine, dried over magnesium sulfate and evaporated. The residue was purified by column chromatography on silica gel (hexane/ethyl acetate=5/1) to give ethyl 3-methoxy-4-(4,4,5,5-tetramethyl-1,3,2-dioxaborolan-2-yl)benzoate (700 mg).

(+)ESI-MS (m/z): 293 (M+1)⁺

Preparation 38

The following compounds were obtained according to a similar manner to that of Preparation 37.

### (1) Methyl 3-chloro-4-(4,4,5,5-tetramethyl-1,3,2-dioxaborolan-2-yl)benzoate

(+)ESI-MS (m/z): 297 (M+1)⁺

### (2) Methyl 3-fluro-4-(4,4,5,5-tetramethyl-1,3,2-dioxaborolan-2-yl)benzoate

NMR (CDCl₃, δ): 1.37 (12H, s), 3.93 (3H, s), 7.61-7.87 (3H, m)

Preparation 39

To a suspension of methyl 3-chloro-4-(4,4,5,5-tetramethyl-1,3,2-dioxaborolan-2-yl)benzoate (2.2 g) in acetone (80 ml) and water (80 ml) were added ammonium acetate (1.2 g) and sodium periodate (3.33 g), and the mixture was stirred at room temperature for 15 hours. The mixture was evaporated and the residue was diluted with ethyl acetate. The organic layer was separated, washed with water and brine, dried over magnesium sulfate and evaporated under reduced pressure. The resultant solid was triturated with diisopropyl ether to give 2-chloro-4-(methoxycarbonyl)phenylboronic acid (275 mg).

(+)ESI-MS (m/z): 213 (M−1)⁻

Preparation 40

To a solution of methyl 4-bromo-2-methylbenzoate (6.9 g) in 1,4-dioxane (150 ml) were added bis(pinacolato)diboron (8.03 g), dichlorobis(triphenylphosphine)palladium(II) (1.69 g) and potassium acetate (8.87 g), and the mixture was stirred at 95° C. for 2 hours under nitrogen. The mixture was diluted with ethyl acetate and water. The organic layer was separated, washed with 1N hydrochloric acid and brine, dried over magnesium sulfate and evaporated. To a suspension of the crude product (11 g) in acetone (200 ml) and water (200 ml) were added ammonium acetate (5.1 g) and sodium periodate (14.1 g), and the mixture was stirred at room temperature for 6 hours. The solvent was evaporated, and the mixture was diluted with ethyl acetate. The organic layer was separated, washed with water and brine, dried over magnesium sulfate and evaporated under reduced pressure. The resultant solid was triturated with diisopropyl ether to give 3-methyl-4-(methoxycarbonyl)phenylboronic acid (2.65 g).

(+)ESI-MS (m/z): 193 (M−1)⁻

Preparation 41

The following compound was obtained according to a similar manner to that of Preparation 40.

### 3-Chloro-4-(methoxycarbonyl)phenylboronic Acid

NMR (DMSO-d₆, δ): 3.86 (3H, s), 7.76 (1H, d, J=3.8 Hz), 7.80 (1H, d, J=3.8 Hz), 8.46 (2H, s)

(−)ESI-MS (m/z): 213 (M−1)⁻

Preparation 42

To an ice-cooled solution of methyl 3-fluoro-4-hydroxybenzoate (10.14 g) and 2,6-lutidine (8.28 g) in dichloromethane (81 ml) was added dropwise trifluoromethanesulfonic anhydride (18.4 g) for 5 minutes, and the mixture was stirred at the same temperature for 30 minutes. The mixture was partitioned between chloroform and water. The organic layer was separated, washed successively with water and brine, dried over magnesium sulfate, and filtered. The filtrate was concentrated and the residue was purified by column chromatography (silica gel, hexane/ethyl acetate) to give methyl 3-fluoro-4-[[(trifluoromethyl)-sulfonyl]oxy]benzoate (16.95 g) as a colorless oil.

NMR (CDCl₃, δ): 3.95 (3H, s), 7.43 (1H, dd, J_{F-H}=8, J_{H-H}=8 Hz), 7.83-8.03 (2H, m)

Preparation 43

The following compounds were obtained according to a similar manner to that of Preparation 12.

### (1) 2-Fluoro-4-(methoxycarbonyl)phenylboronic Acid

NMR (DMSO-d₆, δ): 3.87 (3H, s), 7.50-7.82 (3H, m), 8.47 (2H, br s)

### (2) (7S)-7-[[(Benzyloxy)carbonyl]amino]-5,6,7,8-tetrahydro-2-naphthalenylboronic Acid

(−)ESI-MS (m/z): 324 (M−1)⁻

### (3) 3-Fluoro-4-(methoxycarbonyl)phenylboronic Acid

(+)ESI-MS (m/z): 197 (M−1)⁻

### (4) 4-(Ethoxycarbonyl)-2-methoxyphenylboronic Acid

(+)ESI-MS (m/z): 223 (M−1)⁻

Preparation 44

To a solution of benzyl(2S)-7-hydroxy-1,2,3,4-tetrahydro-2-naphthalenylcarbamate (3.2 g) in dichloromethane (48 ml) were added 4-[(tert-butoxycarbonyl)-amino]-3-(methoxycarbonyl)phenylboronic acid (3.49 g), copper(II)acetate (2.93 g), pyridine (4.35 ml) and dried molecular sieves 4A (3.2 g). The reaction mixture was stirred at room temperature for 16 hours. The precipitate was filtered through a pad of Celite® and the filtrate was concentrated under reduced pressure. The residue was purified by column chromatography on silica gel with ethyl acetate and hexane (1:3 to 1:2) to give methyl 5-[[(7S)-7-[[(benzyloxy)carbonyl]amino]-5,6,7,8-tetrahydro-2-naphthalenyl]oxy]-2-[(tert-butoxycarbonyl)amino]benzoate (3.5 g) as a yellow solid.

(+)ESI-MS (m/z): 569 (M+Na)⁺

Preparation 45

To a solution of methyl 5-[[(7S)-7-[[(benzyloxy)-carbonyl]amino]-5,6,7,8-tetrahydro-2-naphthalenyl]oxy]-2-[(tert-butoxycarbonyl)amino]benzoate (250 mg) in dioxane (1 ml) was added 4N hydrogen chloride in 1,4-dioxane (2.5 ml) and the solution was stirred at room temperature for 3 hours. The mixture was concentrated under reduced pressure. To the residue were added ethyl acetate and aqueous sodium bicarbonate and the mixture was stirred at room temperature for 20 minutes. The organic layer was separated and the aqueous layer was extracted with ethyl acetate. The combined organic layer was dried over magnesium sulfate, filtrated and concentrated under reduced pressure to give methyl 2-amino-5-[[(7S)-7-[[(benzyloxy)carbonyl]amino]-5,6,7,8-tetrahydro-2-naphthalenyl]oxy]benzoate (194 mg) as a yellow oil.

(+)ESI-MS (m/z): 469 (M+Na)⁺

Preparation 46

A tetrahydrofuran solution (1.5 ml) of 2,5-dimethoxytetrahydrofuran (0.29 ml) and 2.5M sulfuric acid (1.12 ml) was added dropwise to a solution of methyl 2-amino-5-[[(7S)-7-[[(benzyloxy)carbonyl]amino]-5,6,7,8-tetrahydro-2-naphthalenyl]oxy]benzoate (500 mg) in a mixture of methanol (2.2 ml) and tetrahydrofuran (2.2 ml) and then sodium borohydride (169 mg) was added portionwise under ice bath. The mixture was stirred at room temperature for 18 hours. The mixture was diluted with water and alkalinized with 3N sodium hydroxide solution. The mixture was extracted with ether and washed with brine. The extract was dried over magnesium sulfate, filtrated and concentrated under reduced pressure. The residue was purified by column chromatography on silica gel with ethyl acetate and hexane (1:4 to 1:3) to give methyl 5-[[(7S)-7-[[(benzyloxy)-carbonyl]amino]-5,6,7,8-tetrahydro-2-naphthalenyl]oxy]-2-(1-pyrrolidinyl)benzoate (443 mg) as a colorless oil.

(+)ESI-MS (m/z): 501 (M+1)⁺

### EXAMPLE 27

The following compounds were obtained according to a similar manner to that of Preparation 4.

### (1) Methyl 4-[(7S)-7-[N-(tert-butoxycarbonyl)-N-[(2R)-2-(3-chlorophenyl)-2-hydroxyethyl]amino]-5,6,7,8-tetrahydro-2-naphthalenyl]-3-methylbenzoate

(+)ESI-MS (m/z): 572 (M+Na)⁺

### (2) Tert-Butyl N-[(2R)-2-(3-chlorophenyl)-2-hydroxyethyl]-N-[(2S)-7-(5-formyl-2-thienyl)-1,2,3,4-tetrahydro-2-naphthalenyl]carbamate

(+)ESI-MS (m/z): 512 (M+1)⁺

### (3) Methyl 4-[(7S)-7-[N-(tert-butoxycarbonyl)-N-[(2R)-2-(3-chlorophenyl)-2-hydroxyethyl]amino]-5,6,7,8-tetrahydro-2-naphthalenyl]-2-methoxybenzoate

(+)ESI-MS (m/z): 566 (M+1)⁺

### (4) Methyl 4-[(7S)-7-[N-(tert-butoxycarbonyl)-N-[(2R)-2-(3-chlorophenyl)-2-hydroxyethyl]amino]-5,6,7,8-tetrahydro-2-naphthalenyl]-2-fluorobenzoate

(+)ESI-MS (m/z): 553 (M+1)⁺

### (5) Ethyl 4-[(7S)-7-[N-(tert-butoxycarbonyl)-N-[(2R)-2-(3-chlorophenyl)-2-hydroxyethyl]amino]-5,6,7,8-tetrahydro-2-naphthalenyl]-3-methoxybenzoate

(+)ESI-MS (m/z): 580 (M+1)⁺

### (6) Ethyl 3-[(7S)-7-[N-(tert-butoxycarbonyl)-N-[(2R)-2-(4-chlorophenyl)-2-hydroxyethyl]amino]-5,6,7,8-tetrahydro-2-naphthalenyl]benzoate

MS (m/z): 572 (M+Na)

### (7) Methyl 4-[(7S)-7-[N-(tert-butoxycarbonyl)-N-[(2R)-2-(4-chlorophenyl)-2-hydroxyethyl]amino]-5,6,7,8-tetrahydro-2-naphthalenyl]-3-methylbenzoate

MS (m/z): 550 (M+1)

### (8) tert-Butyl N-[(2R)-2-(4-chlorophenyl)-2-hydroxyethyl]-N-[(2S)-7-(4-fluoro-3-formylphenyl)-1,2,3,4-tetrahydro-2-naphthalenyl]carbamate

MS (m/z): 524 (M+1)

### (9) Methyl 4-[(7S)-7-[N-benzyl-N-[(2R)-2-hydroxy-2-(6-methyl-3-pyridyl)ethyl]amino]-5,6,7,8-tetrahydro-2-naphthalenyl]benzoate

MS (m/z): 507 (M+1)

### (10) Methyl 4-[(7S)-7-[N-(tert-butoxycarbonyl)-N-[(2R)-2-(5,6-dichloro-3-pyridyl)-2-hydroxyethyl]amino]-5,6,7,8-tetrahydro-2-naphthalenyl]benzoate

MS (m/z): 571 (M+1)

### (11) Methyl 4-[(7S)-7-[N-(tert-butoxycarbonyl)-N-[(2R)-2-(4-chlorophenyl)-2-hydroxyethyl]amino]-5,6,7,8-tetrahydro-2-naphthalenyl]-2-fluorobenzoate

MS (m/z): 554 (M+1)

### (12) Methyl 4-[(7S)-7-[N-(tert-butoxycarbonyl)-N-[(2R)-2-(4-chlorophenyl)-2-hydroxyethyl]amino]-5,6,7,8-tetrahydro-2-naphthalenyl]-3-fluorobenzoate

MS (m/z): 554 (M+1)

### (13) Methyl 4-[(7S)-7-[N-(tert-butoxycarbonyl)-N-[(2R)-2-(4-chlorophenyl)-2-hydroxyethyl]amino]-5,6,7,8-tetrahydro-2-naphthalenyl]-2-chlorobenzoate

MS (m/z): 570 (M+1)

### (14) Methyl 4-[(7S)-7-[N-(tert-butoxycarbonyl)-N-[(2R)-2-(4-chlorophenyl)-2-hydroxyethyl]amino]-5,6,7,8-tetrahydro-2-naphthalenyl]-3-chlorobenzoate

MS (m/z): 570 (M+1)

### (15) Methyl 4-[(7S)-7-[N-(tert-butoxycarbonyl)-N-[(2R)-2-(4-chlorophenyl)-2-hydroxyethyl]amino]-5,6,7,8-tetrahydro-2-naphthalenyl]-2-methylbenzoate

MS (m/z): 550 (M+1)

### (16) Methyl 4-[(7S)-7-[N-(tert-butoxycarbonyl)-N-[(2R)-2-hydroxy-2-(4-methylphenyl)ethyl]amino]-5,6,7,8-tetrahydro-2-naphthalenyl]benzoate

MS (m/z): 516 (M+1)

### (17) tert-Butyl N-[(2R)-2-(4-chlorophenyl)-2-hydroxyethyl]-N-[(2S)-7-(3-formyl-4-methoxyphenyl)-1,2,3,4-trahydro-2-naphthalenyl]carbamate

MS (m/z): 536 (M+1)

### (18) tert-Butyl N-[(2R)-2-(4-chlorophenyl)-2-hydroxyethyl]-N-[(2S)-7-(4-formylphenyl)-1,2,3,4-trahydro-2-naphthalenyl]carbamate

MS (m/z): 506 (M+1)

### EXAMPLE 28

The following compound was obtained according to a similar manner to that of Example 25.

### Ethyl 3-[[(7S)-7-[N-(tert-butoxycarbonyl)-N-[(2R)-2-hydroxy-2-(3-pyridyl)ethyl]amino]-5,6,7,8-tetrahydro-2-naphthalenyl]oxy]benzoate

MS (m/z): 533 (M+1)

### EXAMPLE 29

The following compounds were obtained according to a similar manner to that of Preparation 17.

### (1) Ethyl 3-[[(7S)-7-[[(2R)-2-hydroxy-2-(6-methyl-3-pyridyl)ethyl]amino]-5,6,7,8-tetrahydro-2-naphthalenyl]oxy]benzoate

MS (m/z): 447 (M+1)

### (2) Methyl 4-[(7S)-7-[[(2R)-2-hydroxy-2-(6-methyl-3-pyridyl)ethyl]amino]-5,6,7,8-tetrahydro-2-naphthalenyl]benzoate

MS (m/z): 417 (M+1)

### EXAMPLE 30

The following compound was obtained according to a similar manner to that of Example 17.

### Ethyl 6-[(7S)-7-[[(2R)-2-(3-chlorophenyl)-2-hydroxyethyl]amino]-5,6,7,8-tetrahydro-2-naphthalenyl]-nicotinate

(+)ESI-MS (m/z): 451 (M+1)⁺

### EXAMPLE 31

The following compounds were obtained according to a similar manner to that of Preparation 19.

### (1) Methyl 4-[(7S)-7-[[(2R)-2-(6-chloro-3-pyridyl)-2-hydroxyethyl]amino]-5,6,7,8-tetrahydro-2-naphthalenyl]benzoate

MS (m/z): 437 (M+1)

### (2) Ethyl 4-[(7S)-7-[[(2R)-2-(4-chlorophenyl)-2-hydroxyethyl]amino]-5,6,7,8-tetrahydro-2-naphthalenyl]-3-methoxybenzoate

MS (m/z): 480 (M+1)

### (3) Ethyl 1-[(7S)-7-[[(2R)-2-(4-chlorophenyl)-2-hydroxyethyl]amino]-5,6,7,8-tetrahydro-2-naphthalenyl]-4-piperidinecarboxylate

MS (m/z): 456 (M+1)

### EXAMPLE 32

The following compound was obtained according to a similar manner to that of Preparation 21.

### Ethyl 6-[(7S)-7-[N-(tert-butoxycarbonyl)-N-[(2R)-2-(3-chlorophenyl)-2-hydroxyethyl]amino]-5,6,7,8-tetrahydro-2-naphthalenyl]nicotinate

(+)ESI-MS (m/z): 573 (M+Na)⁺

### EXAMPLE 33

To a solution of tert-butyl N-[(2R)-2-(3-chlorophenyl)-2-hydroxyethyl]-N-[(2S)-7-hydroxy-1,2,3,4-tetrahydro-2-naphthalenyl]carbamate in dichloromethane (300 mg) were added 3-formyl-4-methoxyphenylboronic acid (194 mg), copper(II)acetate (143 mg), pyridine (0.5 ml) and molecular sieves 4A (600 mg). The reaction mixture was stirred at room temperature for 16 hours. The precipitate was filtered through a pad of Celite® and the filtrate was concentrated under reduced pressure. The residue was purified by column chromatography on silica gel with ethyl acetate and hexane (1:3 to 1:2) to give tert-butyl N-[(2R)-2-(3-chlorophenyl)-2-hydroxyethyl]-N-[(2S)-7-(3-formyl-4-methoxyphenoxy)-1,2,3,4-tetrahydro-2-naphthalenyl]carbamate (80 mg) as a white solid.

(+)ESI-MS (m/z): 574 (M+Na)⁺

### EXAMPLE 34

The following compounds were obtained according to a similar manner to that of Example 33.

### (1) tert-Butyl N-[(2R)-2-(3-chlorophenyl)-2-hydroxyethyl]-N-[(2S)-7-(4-fluoro-3-formylphenoxy)-1,2,3,4-tetrahydro-2-naphthalenyl]carbamade

(+)ESI-MS (m/z): 562 (M+Na)⁺

### (2) Methyl 5-[[(7S)-7-[N-(tert-butoxycarbonyl)-N-[2R)-2-(3-chlorophenyl)-2-hydroxyethyl]amino]-5,6,7,8-tetrahydro-2-naphthalenyl]oxy]-2-[[tert-butyl-(dimethyl)silyl]oxy]benzoate

(+)ESI-MS (m/z): 704 (M+Na)⁺

### (3) Methyl 3-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]-5-methoxybenzoate

(+)ESI-MS (m/z): 604 (M+Na)⁺

### (4) Methyl 3-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]-5-nitrobenzoate

(+)ESI-MS (m/z): 619 (M+Na)⁺

### (5) Tert-Butyl N-[(2R)-2-(3-chlorophenyl)-2-hydroxyethyl]-N-[(2S)-7-(5-formyl-2-methoxyphenoxy)-1,2,3,4-tetrahydro-2-naphthalenyl]carbamade

(+)ESI-MS (m/z): 576 (M+Na)⁺

### (6) Methyl 5-([(7S)-7-[N-(tert-butoxycarbonyl)-N-[(2R)-2-(3-chlorophenyl)-2-hydroxyethyl]amino)-5,6,7,8-tetrahydro-2-naphthalenyl]oxy]-2-cyanobenzoate

(+)ESI-MS (m/z): 599 (M+Na)⁺

### (7) Methyl 5-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]-2-methylbenzoate

(+)ESI-MS (m/z): 588 (M+Na)⁺

### (8) Methyl 2-[(tert-butoxycarbonyl)amino]-5-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]-oxy]benzoate

(+)ESI-MS (m/z): 689 (M+Na)⁺

### (9) Methyl 3-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]-5-[[(tert-butyl(dimethyl)silyl]oxy]benzoate

(+)ESI-MS (m/z): 704 (M+Na)⁺

### (10) Methyl 5-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]-2-[N-(tert-butoxycarbonyl)-N-methylamino]benzoate

(+)ESI-MS (m/z): 703 (M+Na)⁺

### (11) Methyl 2-(acetylamino)-5-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]benzoate

(+)ESI-MS (m/z): 631 (M+Na)⁺

### (12) Methyl 5-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]-2-[(methylsulfonyl)-amino]benzoate

(+)ESI-MS (m/z): 667 (M+Na)⁺

### (13) Methyl 5-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]-2-[(ethoxycarbonyl)-amino]benzoate

(+)ESI-MS (m/z): 661 (M+Na)⁺

### (14) Methyl 2-[N-acetyl-N-methylamino]-5-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]benzoate

(+)ESI-MS (m/z): 645 (M+Na)⁺

### (15) Methyl 2-(benzoylamino)-5-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]-oxy]benzoate

(+)ESI-MS (m/z): 693 (M+Na)⁺

### (16) Methyl 5-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]-2-[(2,2-dimethylpropanoyl)amino]benzoate

(+)ESI-MS (m/z): 673 (M+Na)⁺

### (17) Methyl 5-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]-2-(2-oxo-1-pyrrolidinyl)benzoate

(+)ESI-MS (m/z): 657 (M+Na)⁺

### (18) Ethyl 3-[[(7S)-7-[N-benzyl-N-[(2R)-2-hydroxy-2-(6-methyl-3-pyridyl)ethyl]amino]-5,6,7,8-tetrahydro-2-naphthalenyl]oxy]benzoate

MS (m/z): 537 (M+1)

### (19) Ethyl 3-[[(7S)-7-[N-(tert-butoxycarbonyl)-N-[(2R)-2-(6-chloro-3-pyridyl)-2-hydroxyethyl]amino]-5,6,7,8-tetrahydro-2-naphthalenyl]oxy]benzoate

MS (m/z): 567 (M+1)

### (20) Tert-Butyl N-[(2R)-2-(4-chlorophenyl)-2-hydroxyethyl]-N-[(2S)-7-(3-formyl-4-methoxyphenoxy)-1,2,3,4-tetrahydro-2-naphthalenyl]carbamate

MS (m/z): 574 (M+Na)

### (21) Ethyl 3-[[(7S)-7-[N-(tert-butoxycarbonyl)-N-[(2R)-2-(4-chlorophenyl)-2-hydroxyethyl]amino]-5,6,7,8-tetrahydro-2-naphthalenyl]oxy]benzoate

MS (m/z): 588 (M+Na)

### (22) Methyl 4-[[(7S)-7-[N-(tert-butoxycarbonyl)-N-[(2R)-2-(4-chlorophenyl)-2-hydroxyethyl]amino]-5,6,7,8-tetrahydro-2-naphthalenyl]oxy]benzoate

MS (m/z): 574 (M+Na)

### (23) Methyl 4-[[(7S)-7-[N-(tert-butoxycarbonyl)-N-[(2R)-2-(4-chlorophenyl)-2-hydroxyethyl]amino]-5,6,7,8-tetrahydro-2-naphthalenyl]oxy]-2-methoxybenzoate

MS (m/z): 582 (M+1)

### (24) Methyl 5-[[(7S)-7-[N-(tert-butoxycarbonyl)-N-[(2R)-2-(4-chlorophenyl)-2-hydroxyethyl]amino]-5,6,7,8-tetrahydro-2-naphthalenyl]oxy]-2-chlorobenzoate

MS (m/z): 586 (M+1)

### (25) Ethyl 3-[[(7S)-7-[N-(tert-butoxycarbonyl)-N-[(2R)-2-(5,6-dichloro-3-pyridyl)-2-hydroxyethyl]amino]-5,6,7,8-tetrahydro-2-naphthalenyl]oxy]benzoate

MS (m/z): 601 (M+1)

### (26) Tert-Butyl N-[(2R)-2-(6-chloro-3-pyridyl)-2-hydroxyethyl]-N-[(2S)-7-(3-formyl-4-methoxyphenoxy)-1,2,3,4-tetrahydro-2-naphthalenyl]carbamate

MS (m/z): 553 (M+1)

### (27) Ethyl 3-[[(7S)-7-[N-(tert-butoxycarbonyl)-N-[(2R)-2-hydroxy-2-(4-methylphenyl)ethyl]amino]-5,6,7,8-tetrahydro-2-naphthalenyl]oxy]benzoate

MS (m/z): 546 (M+1)

### (28) Methyl 5-[[(7S)-7-[N-(tert-butoxycarbonyl)-N-[(2R)-2-(6-chloro-3-pyridyl)-2-hydroxyethyl]amino]-5,6,7,8-tetrahydro-2-naphthalenyl]oxy]-2-chlorobenzoate

MS (m/z): 587 (M+1)

### (29) Methyl 5-[[(7S)-7-[N-(tert-butoxycarbonyl)-N-[(2R)-2-hydroxy-2-(6-methyl-3-pyridyl)ethyl]amino]-5,6,7,8-tetrahydro-2-naphthalenyl]oxy]-2-chlorobenzoate

MS (m/z): 567 (M+1)

### (30) Tert-Butyl N-[(2R)-2-(5,6-dichloro-3-pyridyl)-2-hydroxyethyl]-N-[(2S)-7-(3-formyl-4-methoxyphenoxy)-1,2,3,4-tetrahydro-2-naphthalenyl]carbamate

MS (m/z): 587 (M+1)

### EXAMPLE 35

To a solution of tert-butyl N-[(2R)-2-(3-chlorophenyl)-2-hydroxyethyl]-N-[(2S)-7-(3-formyl-4-methoxyphenoxy)-1,2,3,4-tetrahydro-2-naphthalenyl]carbamate (80 mg) in a mixture of acetonitrile (1 ml) and water (0.3 ml) were added 35% solution of hydrogen peroxide in water (28 μl) and potassium dihydrogenphosphate (78.9 mg). After cooling to 4° C., a solution of sodium chlorite (26.2 mg) in water (0.3 ml) was added dropwise to the solution. The solution was stirred at room temperature for 1 hour. To the solution was added sodium sulfite (73.1 mg) at 4° C. After adding 1M citric acid aqueous solution, the solution was extracted with ethyl acetate. The organic layer was separated and washed with water and brine. The extract was dried over magnesium sulfate and concentrated under reduced pressure. The residue was purified by column chromatography on silica gel with chloroform and methanol (100:0 to 90:10) to give 5-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]-2-methoxybenzoic acid (46.8 mg) as a white solid.

(−)ESI-MS (m/z): 566 (M-1)⁻

### EXAMPLE 36

The following compounds were obtained according to a similar manner to that of Example 35.

### (1) 5-[[(7S)-7-[N-(tert-Butoxycarbonyl)-N-[(2R)-2-(3-chlorophenyl)-2-hydroxyethyl]amino]-5,6,7,8-tetrahydro-2-naphthalenyl]oxy]-2-fluorobenzoic Acid

(−)ESI-MS (m/z): 554 (M−1)⁻

### (2) 3-[[(7S)-7-[N-(tert-Butoxycarbonyl)-N-[(2R)-2-(3-chlorophenyl)-2-hydroxyethyl]amino]-5,6,7,8-tetrahydro-2-naphthalenyl]oxy]-4-methoxybenzoic Acid

(−)ESI-MS (m/z): 566 (M−1)⁻

### (3) 5-[[(7S)-7-[N-(tert-Butoxycarbonyl)-N-[(2R)-2-(3-chlorophenyl)-2-hydroxyethyl]amino]-5,6,7,8-tetrahydro-2-naphthalenyl]oxy]-2-phenoxybenzoic Acid

(−)ESI-MS (m/z): 628 (M−1)⁻

### (4) 5-[(7S)-7-[(N-(tert-Butoxycarbonyl)-N-[(2R)-2-(3-chlorophenyl)-2-hydroxyethyl]amino]-5,6,7,8-tetrahydro-2-naphthalenyl]-2-thiophenecarboxylic Acid

(−)ESI-MS (m/z): 526 (M−1)⁻

### (5) 5-[[(7S)-7-[N-(tert-Butoxycarbonyl)-N-[(2R)-2-(4-chlorophenyl)-2-hydroxyethyl]amino]-5,6,7,8-tetrahydro-2-naphthalenyl]oxy]-2-methoxybenzoic Acid

MS (m/z): 568 (M+1)

### (6) 5-[(7S)-7-[N-(tert-Butoxycarbonyl)-N-[(2R)-2-(4-chlorophenyl)-2-hydroxyethyl]amino]-5,6,7,8-tetrahydro-2-naphthalenyl]-2-fluorobenzoic Acid

MS (m/z): 540 (M+1)

### (7) 5-[[(7S)-7-[N-(tert-Butoxycarbonyl)-N-[(2R)-2-(6-chloro-3-pyridyl)-2-hydroxyethyl]amino]-5,6,7,8-tetrahydro-2-naphthalenyl]oxy]-2-methoxybenzoic Acid

MS (m/z): 569 (M+1)

### (8) 5-[[(7S)-7-[N-(tert-Butoxycarbonyl)-N-[(2R)-2-(5,6-dichloro-3-pyridyl)-2-hydroxyethyl]amino]-5,6,7,8-tetrahydro-2-naphthalenyl]oxy]-2-methoxybenzoic Acid

MS (m/z): 603 (M+1)

### (9) 5-[(7S)-7-[N-(tert-Butoxycarbonyl)-N-[(2R)-2-(4-chlorophenyl)-2-hydroxyethyl]amino]-5,6,7,8-tetrahydro-2-naphthalenyl]-2-methoxybenzoic Acid

MS (m/z): 552 (M+1)

### EXAMPLE 37

To a solution of 5-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]-2-methoxybenzoic acid (46.8 mg) in 1,4-dioxane (0.2 ml) was added 4N hydrogen chloride in 1,4-dioxane (1 ml) dropwise. The solution was stirred at room temperature for 3 hours. The solution was concentrated under reduced pressure to give 5-[[(7S)-7-[[(2R)-2-(3-chlorophenyl)-2-hydroxyethyl]amino]-5,6,7,8-tetrahydro-2-naphthalenyl]oxy]-2-methoxybenzoic acid hydrochloride (41.0 mg) as a white solid.

NMR (DMSO-d₆, δ): 1.79-1.91 (1H, m), 2.28-2.33 (1H, m), 2.77-2.91 (2H, m), 3.16-3.61 (5H, m), 3.80 (3H, s), 5.04-5.08 (1H, m), 6.34-6.36 (1H, m), 6.69-7.50 (10H, m), 8.94 (1H, br s), 9.40 (1H, br s), 12.72 (1H, br s)

(+)ESI-MS (m/z): 482 (M−HCl+Na)⁺

### EXAMPLE 38

The following compounds were obtained according to a similar manner to that of Example 37.

### (1) 2-Chloro-5-[[(7S)-7-[[(2R)-2-(3-chlorophenyl)-2-hydroxyethyl]amino]-5,6,7,8-tetrahydro-2-naphthalenyl]oxy]benzoic Acid Hydrochloride

NMR (DMSO-d₆, δ): 1.12-1.28 (1H, m), 1.83-1.91 (2H, m), 2.32-2.57 (1H, m), 2.83-3.13 (2H, m), 3.24-3.56 (2H, m), 3.64-3.73 (1H, m), 5.09-5.13 (1H, m), 6.38 (1H, m), 6.84-7.71 (10H, m), 9.03 (1H, br s), 9.61 (1H, br s), 13.38 (1H, br s)

(−)ESI-MS (m/z): 470 (M−HCl−1)⁻

### (2) 5-[[(7S)-7-[[(2R)-2-(3-Chlorophenyl)-2-hydroxyethyl]-amino]-5,6,7,8-tetrahydro-2-naphthalenyl]oxy]-2-fluorobenzoic Acid Hydrochloride

NMR (DMSO-d₆, δ): 1.14-1.35 (1H, m), 1.83-1.86 (2H, m), 2.28-2.52 (1H, m), 2.92-3.10 (2H, m), 3.22-3.68 (3H, m), 5.03-5.08 (1H, m), 6.35-6.37 (1H, m), 6.78-6.89 (2H, m), 7.14-7.50 (8H, m), 8.92 (1H, br s), 9.34 (1H, br s), 13.41 (1H, br s)

(−)ESI-MS (m/z): 454 (M−HCl−1)⁻

### (3) 3-[[(7S)-7-[[(2R)-2-(3-Chlorophenyl)-2-hydroxyethyl]-amino]-5,6,7,8-tetrahydro-2-naphthalenyl]oxy]-4-methoxybenzoic Acid Hydrochloride

NMR (DMSO-d₆, δ): 1.23-1.28 (1H, m), 1.78-1.84 (2H, m), 2.24-2.29 (1H, m), 2.74-2.83 (2H, m), 3.11-3.64 (3H, m), 3.83 (3H, s), 4.98-5.03 (1H, m), 6.33 (1H, m), 6.63-6.76 (2H, m), 7.07-7.50 (7H, m), 7.77 (1H, dd, J=2, 8 Hz), 8.89=9.09 (2H, br), 12.74 (1H, br s)

(−)ESI-MS (m/z): 466 (M−HCl−1)⁻

### (4) 5-[[(7S)-7-[[(2R)-2-(3-Chlorophenyl)-2-hydroxyethyl]-amino]-5,6,7,8-tetrahydro-2-naphthalenyl]oxy]-2-phenoxybenzoic Acid Hydrochloride

NMR (DMSO-d₆, δ): 1.52-1.56 (1H, m), 1.72-1.86 (2H, m), 2.29-2.35 (1H, m), 2.78-2.95 (2H, m), 3.11-3.68 (3H, m), 5.03-5.08 (1H, m), 6.34-6.36 (1H, m), 6.82-7.50 (15H, m), 8.94 (1H, br s), 9.29 (1H, br s), 12.90 (1H, br s)

(+)ESI-MS (m/z): 530 (M−HCl+1)⁺

### (5) 5-[(7S)-7-[[(2R)-2-(3-Chlorophenyl)-2-hydroxyethyl]-amino]-5,6,7,8-tetrahydro-2-naphthalenyl]-2-thiophenecarboxylic Acid Hydrochloride

NMR (DMSO-d₆, δ): 1.74-1.77 (1H, m), 1.80-1.95 (1H, m), 2.30-2.33 (1H, m), 2.80-2.95 (3H, m), 3.13-3.16 (1H, m), 3.29-3.36 (1H, m), 3.52-3.62 (2H, m), 5.04 (1H, d, J=9.2 Hz), 6.36 (1H, br), 7.20 (1H, d, J=8.0 Hz), 7.39-7.53 (7H, m), 7.71 (1H, d, J=4.0 Hz), 9.01 (1H, br), 13.1 (1H, br)

(−)ESI-MS (m/z): 426 (M−HCl−1)⁻

### (6) 3-[[(7S)-7-[[(2R)-2-Hydroxy-2-(3-pyridyl)ethyl]amino]-5,6,7,8-tetrahydro-2-naphthalenyl]oxy]benzoic Acid Dihydrochloride

NMR (DMSO-d₆, δ): 1.90-2.05 (1H, m), 2.30-2.40 (1H, m), 2.70-3.10 (3H, m), 3.20-3.60 (4H, m), 5.30-5.45 (1H, m), 6.80-6.95 (2H, m), 7.10-7.70 (6H, m), 8.00 (1H, dd, J=5, 8 Hz), 8.60 (1H, d, J=8 Hz), 8.85 (1H, d, J=5 Hz)

MS (m/z): 405 (M+1)

### (7) 3-[[(7S)-7-[[(2R)-2-(6-Chloro-3-pyridyl)-2-hydroxyethyl]amino]-5,6,7,8-tetrahydro-2-naphthalenyl]oxy]benzoic Acid Hydrochloride

NMR (DMSO-d₆, δ): 1.80-1.90 (1H, m), 2.30-2.40 (1H, m), 2.50-3.50 (7H, m), 5.10-5.20 (1H, m), 6.80-7.00 (2H, m), 7.15-7.70 (6H, m), 7.90-8.00 (1H, m), 8.48 (1H, s)

MS (m/z): 439 (M+1)

### (8) 3-[[(7S)-7-[[(2R)-2-(4-Chlorophenyl)-2-hydroxyethyl]-amino]-5,6,7,8-tetrahydro-2-naphthalenyl]oxy]benzoic Acid Hydrochloride

NMR (DMSO-d₆, δ): 1.85-2.05 (1H, m), 2.30-2.50 (1H, m), 2.70-3.60 (7H, m), 5.10-5.20 (1H, m), 6.80-6.90 (2H, m), 7.20-7.80 (9H, m)

MS (m/z): 43.8 (M+1)

### (9) 5-[[(7S)-7-[[(2R)-2-(4-Chlorophenyl)-2-hydroxyethyl]-amino]-5,6,7,8-tetrahydro-2-naphthalenyl]oxy]-2-methoxybenzoic Acid Hydrochloride

NMR (DMSO-d₆, δ): 1.75-2.00 (1H, m), 2.20-2.40 (1H, m), 2.60-3.60 (7H, m), 3.80 (3H, s), 5.05-5.15 (1H, m), 6.75-6.90 (2H, m), 7.05-7.25 (4H, m), 7.40-7.50 (4H, m)

MS (m/z): 468 (M+1)

### (10) 3-[(7S)-7-[[(2R)-2-(4-Chlorophenyl)-2-hydroxyethyl]-amino]-5,6,7,8-tetrahydro-2-naphthalenyl]benzoic Acid Hydrochloride

NMR (DMSO-d₆, δ): 1.80-2.05 (1H, m), 2.30-2.50 (1H, m), 2.70-3.60 (7H, m), 5.10-5.20 (1H, m), 7.20 (1H, d, J=8 Hz), 7.40-7.75 (7H, m), 7.90 (2H, t, J=8 Hz), 8.18 (1H, s)

MS (m/z): 422 (M+1)

### (11) 4-[[(7S)-7-[[(2R)-2-(4-Chlorophenyl)-2-hydroxyethyl]-amino]-5,6,7,8-tetrahydro-2-naphthalenyl]oxy]benzoic Acid Hydrochloride

NMR (DMSO-d₆, δ): 1.75-2.00 (1H, m), 2.30-2.45 (1H, m), 2.70-3.60 (7H, m), 5.00-5.10 (1H, m), 6.80-7.00 (4H, m), 7.20 (1H, d, J=8 Hz), 7.40-7.50 (4H, m), 7.90 (1H, d, J=8 Hz)

MS (m/z): 438 (M+1)

### (12) 4-[[(7S)-7-[[(2R)-2-(4-Chlorophenyl)-2-hydroxyethyl]-amino]-5,6,7,8-tetrahydro-2-naphthalenyl]oxy]-2-methoxybenzoic Acid Hydrochloride

NMR (DMSO-d₆, δ): 1.85-2.00 (1H, m), 2.30-2.40 (1H, m), 2.70-3.60 (7H, m), 3.78 (3H, s), 5.00-5.10 (1H, m), 6.40-6.50 (1H, m), 6.70-6.95 (3H, m), 7.20 (1H, d, J=8 Hz), 7.40-7.50 (4H, m), 7.70 (1H, d, J=8 Hz).

MS (m/z): 468 (M+1)

### (13) 4-[(7S)-7-[[(2R)-2-(4-Chlorophenyl)-2-hydroxyethyl]-amino]-5,6,7,8-tetrahydro-2-naphthalenyl]-3-methoxybenzoic Acid Hydrochloride

NMR (DMSO-d₆, δ): 1.75-1.85 (1H, m), 2.40 (3H, s), 2.40-2.50 (1H, m), 2.70-3.00 (7H, m), 5.00-5.10 (1H, m), 7.00-7.30 (4H, m), 7.35-7.45 (5H, m), 7.80-7.90 (1H, m)

MS (m/z): 436 (M+1)

### (14) 5-[(7S)-7-[[(2R)-2-(4-Chlorophenyl)-2-hydroxyethyl]-amino]-5,6,7,8-tetrahydro-2-naphthalenyl]-2-fluorobenzoic Acid Hydrochloride

NMR (DMSO-d₆, δ): 1.80-2.00 (1H, m), 2.30-2.40 (1H, m), 2.70-3.60 (7H, m), 5.00-5.10 (1H, m), 7.15-7.50 (8H, m), 7.85-7.95 (1H, m), 8.00-8.10 (1H, m)

MS (m/z): 440 (M+1)

### (15) 2-Chloro-5-[[(7S)-7-[[(2R)-2-(4-chlorophenyl)-2-hydroxyethyl]amino]-5,6,7,8-tetrahydro-2-naphthalenyl]-oxy]benzoic Acid Hydrochloride

NMR (DMSO-d₆, δ): 1.75-1.90 (1H, m), 2.25-2.40 (1H, m), 2.70-3.60 (7H, m), 5.00-5.10 (1H, m), 6.85-6.95 (2H, m), 7.15-7.30 (3H, m), 7.45-7.55 (5H, m)

MS (m/z): 471 (M+1)

### (16) 3-Chloro-2-[[(7S)-7-[[(2R)-2-(4-chlorophenyl)-2-hydroxyethyl]amino]-5,6,7,8-tetrahydro-2-naphthalenyl]-oxy]isonicotinic Acid Hydrochloride

NMR (DMSO-d₆, δ): 1.75-1.90 (1H, m), 2.20-2.40 (1H, m), 2.70-3.70 (7H, m), 5.00-5.10 (1H, m), 6.85-7.20 (3H, m), 7.33 (1H, d, J=5 Hz), 7.40-7.50 (4H, m), 8.10 (1H, d, J=5 Hz)

MS (m/z): 473 (M+1)

### (17) 3-[[(7S)-7-[[(2R)-2-(5,6-Dichloro-3-pyridyl)-2-hydroxyethyl]amino]-5,6,7,8-tetrahydro-2-naphthalenyl]-oxy]benzoic Acid Hydrochloride

NMR (DMSO-d₆, δ): 1.80-1.95 (1H, m), 2.30-2.40 (1H, m), 2.70-3.40 (7H, m), 5.10-5.20 (1H, m), 6.80-6.95 (2H, m), 7.10-7.75 (5H, m), 8.20 (1H, d, J=2 Hz), 8.40 (1H, d, J=2 Hz)

MS (m/z): 473 (M+1)

### (18) 5-[[(7S)-7-[[(2R)-2-(6-Chloro-3-pyridyl)-2-hydroxyethyl]amino]-5,6,7,8-tetrahydro-2-naphthalenyl]-oxy]-2-methoxybenzoic Acid Hydrochloride

NMR (DMSO-d₆, δ): 1.75-1.85 (1H, m), 2.30-2.40 (1H, m), 2.70-3.30 (7H, m), 3.80 (3H, s), 5.00-5.10 (1H, m), 6.65-6.80 (2H, m), 7.00-7.20 (4H, m), 7.55 (1H, d, J=8 Hz), 7.90 (1H, dd, J=2, 8 Hz), 8.45 (1H, d, J=2 Hz)

### (19) 3-[[(7S)-7-[[(2R)-2-Hydroxy-2-(4-methylphenyl]ethyl]-amino]-5,6,7,8-tetrahydro-2-naphthalenyl)oxy]benzoic Acid Hydrochloride

NMR (DMSO-d₆, δ): 1.75-2.00 (2H, m), 2.30 (3H, s), 2.70-3.70 (7H, m), 5.00-5.10 (1H, m), 6.80-6.95 (2H, m), 7.10-7.70 (9H, m)

MS (m/z): 418 (M+1)

### (20) 2-Chloro-5-[[(7S)-7-[[(2R)-2-(6-chloro-3-pyridyl)-2-hydroxyethyl]amino]-5,6,7,8-tetrahydro-2-naphthalenyl]oxy]benzoic Acid Hydrochloride

NMR (DMSO-d₆, δ): 1.80-1.95 (1H, m), 2.30-2.40 (1H, m), 2.70-3.70 (7H, m), 5.10-5.15 (1H, m), 6.85-6.95 (2H, m), 7.15-7.30 (3H, m), 7.50-7.60 (2H, m), 7.90 (1H, dd, J=2, 8 Hz), 8.45 (1H, d, J=2 Hz)

MS (m/z): 473 (M+1)

### (21) 5-[[(7S)-7-[[(2R)-2-(5,6-Dichloro-3-pyridyl)-2-hydroxyethyl]amino]-5,6,7,8-tetrahydro-2-naphthalenyl]oxy]-2-methoxybenzoic Acid Hydrochloride

NMR (DMSO-d₆, δ): 1.75-1.95 (1H, m), 2.25-2.40 (1H, m), 2.70-3.70 (7H, m), 3.90 (3H, s), 5.10-5.20 (1H, m), 6.65-6.85 (2H, m), 7.10-7.30 (4H, m), 8.20 (1H, d, J=2 Hz), 8.45 (1H, d, J=2 Hz)

MS (m/z): 503 (M+1)

### (22) 2-Chloro-5-[[(7S)-7-[[(2R)-2-hydroxy-2-(6-methyl-3-pyridyl)ethyl]amino]-5,6,7,8-tetrahydro-2-naphthalenyl]oxy]benzoic Acid Dihydrochloride

NMR (DMSO-d₆, δ): 1.70-1.95 (1H, m), 2.25-2.40 (1H, m), 2.75 (3H, s), 2.70-3.70 (7H, m), 5.20-5.35 (1H, m), 6.85-6.95 (2H, m), 7.10-7.30 (3H, m), 7.40-7.55 (1H, m), 7.80 (1H, d, J=8 Hz), 8.50 (1H, d, J=8 Hz), 8.80 (1H, s)

MS (m/z): 453 (M+1)

### (23) 4-[(7S)-7-[[(2R)-2-(5,6-Dichloro-3-pyridyl)-2-hydroxyethyl]amino]-5,6,7,8-tetrahydro-2-naphthalenyl]benzoic Acid Hydrochloride

NMR (DMSO-d₆, δ): 1.75-1.95 (1H, m), 2.25-2.40 (1H, m), 2.70-3.60 (7H, m), 5.10-5.20 (1H, m), 7.20 (1H, d, J=8 Hz), 7.40-7.50 (2H, m), 7.70 (1H, d, J=8 Hz), 8.00 (1H, d, J=8 Hz), 8.20 (1H, d, J=2 Hz), 8.50 (1H, d, J=2 Hz)

MS (m/z): 457 (M+1)

### (24) 4-[(7S)-7-[[(2R)-2-(4-Chlorophenyl)-2-hydroxyethyl]-amino]-5,6,7,8-tetrahydro-2-naphthalenyl]-2-fluorobenzoic Acid Hydrochloride

NMR (DMSO-d₆, δ): 1.80-1.95 (1H, m), 2.25-2.40 (1H, m), 2.70-3.60 (7H, m), 5.00-5.10 (1H, m), 7.20 (1H, d, J=8 Hz), 7.40-7.65 (8H, m), 7.90 (1H, t, J=8 Hz)

MS (m/z): 440 (M+1)

### (25) 4-[(7S)-7-[[(2R)-2-(4-Chlorophenyl)-2-hydroxyethyl]-amino]-5,6,7,8-tetrahydro-2-naphthalenyl]-3-fluorobenzoic Acid Hydrochloride

NMR (DMSO-d₆, δ): 1.70-1.95 (1H, m), 2.30-2.40 (1H, m), 2.70-3.50 (7H, m), 5.00-5.10 (1H, m), 7.20-7.90 (10H, m)

MS (m/z): 440 (M+1)

### (26) 2-Chloro-4-[(7S)-7-[[(2R)-2-(4-chlorophenyl)-2-hydroxyethyl]amino]-5,6,7,8-tetrahydro-2-naphthalenyl]-benzoic Acid Hydrochloride

NMR (DMSO-d₆, δ): 1.80-2.00 (1H, m), 2.25-2.40 (1H, m), 2.70-3.70 (7H, m), 5.10-5.20 (1H, m), 7.15-7.20 (1H, m), 7.35-7.90 (9H, m)

MS (m/z): 456 (M+1)

### (27) 3-Chloro-4-[(7S)-7-[[(2R)-2-(4-chlorophenyl)-2-hydroxyethyl]amino]-5,6,7,8-tetrahydro-2-naphthalenyl]-benzoic Acid Hydrochloride

NMR (DMSO-d₆, δ): 1.80-2.00 (1H, m), 2.30-2.40 (1H, m), 2.70-3.40 (7H, m), 5.00-5.15 (1H, m), 7.20-7.30 (2H, m), 7.40-7.60 (6H, m), 7.90-8.05 (2H, m)

MS (m/z): 456 (M+1)

### (28) 4-[(7S)-7-[[(2R)-2-(4-Chlorophenyl)-2-hydroxyethyl]-amino]-5,6,7,8-tetrahydro-2-naphthalenyl]-2-methylbenzoic acid hydrochloride

NMR (DMSO-d₆, δ): 1.80-2.00 (1H, m), 2.30-2.40 (1H, m), 2.59 (3H, s), 2.70-3.40 (7H, m), 5.05-5.15 (1H, m), 7.24 (1H, d, J=8 Hz), 7.30-7.65 (8H, m), 7.90 (1H, d, J=8 Hz)

MS (m/z): 436 (M+1)

### (29) 4-[(7S)-7-[[(2R)-2-Hydroxy-2-(4-methylphenyl)ethyl]-amino]-5,6,7,8-tetrahydro-2-naphthalenyl]benzoic Acid Hydrochloride

NMR (DMSO-d₆, δ): 1.80-2.00 (1H, m), 2.31 (3H, s), 2.25-2.50 (1H, m), 2.70-3.70 (7H, m), 5.00-5.10 (1H, m), 6.85-6.95 (2H, m), 7.10-7.55 (7H, m), 7.80 (2H, d, J=8 Hz), 8.00 (2H, d, J=8 Hz)

MS (m/z): 402 (M+1)

### (30) 5-[(7S)-7-[[(2R)-2-(4-Chlorophenyl)-2-hydroxyethyl]-amino]-5,6,7,8-tetrahydro-2-naphthalenyl]-2-methoxybenzoic Acid Hydrochloride

NMR (DMSO-d₆, δ): 1.70-2.00 (1H, m), 2.25-2.40 (1H, m), 2.70-3.70 (7H, m), 3.85 (3H, s), 5.00-5.15 (1H, m), 7.15-7.35 (2H, m), 7.40-7.60 (6H, m), 7.70-7.90 (2H, m)

MS (m/z): 452 (M+1)

### (31) (2E)-3-[4-[(7S)-7-[[(2R)-2-(4-Chlorophenyl)-2-hydroxyethyl]amino]-5,6,7,8-tetrahydro-2-naphthalenyl)phenyl]-2-propenoic Acid Hydrochloride

NMR (DMSO-d₆, δ): 1.80-2.00 (1H, m), 2.30-2.45 (1H, m), 2.70-3.70 (7H, m), 5.05-5.15 (1H, m), 6.60 (1H, d, J=16 Hz), 7.20 (1H, d, J=8 Hz), 7.40-7.80 (11H, m)

MS (m/z): 448 (M+1)

### EXAMPLE 39

Under nitrogen gas, to a solution of tert-butyl N-[(2R)-2-(3-chlorophenyl)-2-hydroxyethyl]-N-[(2S)-7-hydroxy-1,2,3,4-tetrahydro-2-naphthalenyl]carbamate (500 mg) in toluene (5 ml) was added methyl 5-bromo-2-chlorobenzoate (358 ml), 2-(di-tert-butylphosphino)biphenyl (42.8 mg), potassium phosphate (509 mg) and palladium(II)acetate (32.2 mg) and the mixture was stirred at 100° C. for 17 hours. The reaction mixture was diluted with ethyl acetate and the precipitate was filtered through a pad of Celite®. After concentration under reduced pressure, the residue was purified by column chromatography on silica gel with ethyl acetate and hexane (1:4 to 1:3) to give methyl 5-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]-2-chlorobenzoate (118 mg) as a white solid.

(+)ESI-MS (m/z): 608 (M+Na)⁺

### EXAMPLE 40

To a solution of methyl 5-[[(7S)-7-[N-(tert-butoxycarbonyl)-N-[(2R)-2-(3-chlorophenyl)-2-hydroxyethyl]-amino]-5,6,7,8-tetrahydro-2-naphthalenyl]oxy]-2-chlorobenzoate (118 mg) in methanol (1.2 ml) was added 1N sodium hydroxide (0.4 ml) and the solution was stirred at 60° C. for 1 hour. The solution was cooled to room temperature. To the solution was added 1N hydrochloric acid (0.45 ml) dropwise. The solution was extracted with ethyl acetate and washed with 1N hydrochloric acid and water. The extract was dried over magnesium sulfate, filtrated and concentrated under reduced pressure to give 5-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]-2-chlorobenzoic acid (89.2 mg) as a white solid.

(−)ESI-MS (m/z): 570 (M−1)⁻

### EXAMPLE 41

The following compounds were obtained according to a similar manner to that of Example 40.

### (1) 3-[[(7S)-7-[N-(tert-Butoxycarbonyl)-N-[(2R)-2-hydroxy-2-(3-pyridyl)ethyl]amino]-5,6,7,8-tetrahydro-2-naphthalenyl]oxy]benzoic Acid

MS (m/z): 505 (M+1)

### (2) 3-[[(7S)-7-[N-(tert-Butoxycarbonyl)-N-[(2R)-2-(6-chloro-3-pyridyl)-2-hydroxyethyl]amino]-5,6,7,8-tetrahydro-2-naphthalenyl]oxy]benzoic Acid

MS (m/z): 539 (M+1)

### (3) 3-[[(7S)-7-[N-(tert-Butoxycarbonyl)-N-[(2R)-2-(4-chlorophenyl)-2-hydroxyethyl]amino]-5,6,7,8-tetrahydro-2-naphthalenyl]oxy]benzoic Acid

MS (m/z): 538 (M+1)

### (4) 3-[(7S)-7-[N-(tert-Butoxycarbonyl)-N-[(2R)-2-(4-chlorophenyl)-2-hydroxyethyl]amino]-5,6,7,8-tetrahydro-2-naphthalenyl]benzoic Acid

MS (m/z): 522 (M+1)

### (5) 4-[[(7S)-7-[N-(tert-Butoxycarbonyl)-N-[(2R)-2-(4-chlorophenyl)-2-hydroxyethyl]amino]-5,6,7,8-tetrahydro-2-naphthalenyl]oxy]benzoic Acid

MS (m/z): 536 (M−1)

### (6) 4-[[(7S)-7-[N-(tert-Butoxycarbonyl)-N-[(2R)-2-(4-chlorophenyl)-2-hydroxyethyl]amino]-5,6,7,8-tetrahydro-2-naphthalenyl]oxy]-2-methoxybenzoic Acid

MS (m/z): 568 (M+1)

### (7) 4-[(7S)-7-[N-(tert-Butoxycarbonyl)-N-[(2R)-2-(4-chlorophenyl)-2-hydroxyethyl]amino]-5,6,7,8-tetrahydro-2-naphthalenyl]-3-methylbenzoic Acid

MS (m/z): 536 (M+1)

### (8) 5-[[(7S)-7-[N-(tert-Butoxycarbonyl)-N-[(2R)-2-(4-chlorophenyl)-2-hydroxyethyl]amino]-5,6,7,8-tetrahydro-2-naphthalenyl]oxy]-2-chlorobenzoic Acid

MS (m/z): 572 (M+1)

### (9) 2-[[(7S)-7-[N-(tert-Butoxycarbonyl)-N-[(2R)-2-(4-chlorophenyl)-2-hydroxyethyl]amino]-5,6,7,8-tetrahydro-2-naphthalenyl]oxy]-2-chloroisonicotinic Acid

MS (m/z): 574 (M+1)

### (10) 3-[[(7S)-7-[N-(tert-Butoxycarbonyl)-N-[(2R)-2-(5,6-dichloro-3-pyridyl)-2-hydroxyethyl]amino]-5,6,7,8-tetrahydro-2-naphthalenyl]oxy]benzoic Acid

MS (m/z): 573 (M+1)

### (11) 3-[[(7S)-7-[N-(tert-Butoxycarbonyl)-N-[(2R)-2-hydroxy-2-(4-methylphenyl)ethyl]amino]-5,6,7,8-tetrahydro-2-naphthalenyl]oxy]benzoic Acid

MS (m/z): 518 (M+1)

### (12) 5-[[(7S)-7-[N-(tert-Butoxycarbonyl)-N-[(2R)-2-(6-chloro-3-pyridyl)-2-hydroxyethyl]amino]-5,6,7,8-tetrahydro-2-naphthalenyl]oxy]-2-chlorobenzoic Acid

MS (m/z): 573 (M+1)

### (13) 5-[[(7S)-7-[N-(tert-Butoxycarbonyl)-N-[(2R)-2-hydroxy-2-(6-methyl-3-pyridyl)ethyl]amino]-5,6,7,8-tetrahydro-2-naphthalenyl]oxy]-2-chlorobenzoic Acid

MS (m/z): 553 (M+1)

### (14) 4-[(7S)-7-[N-(tert-Butoxycarbonyl)-N-[(2R)-2-(5,6-dichloro-3-pyridyl)-2-hydroxyethyl]amino]-5,6,7,8-tetrahydro-2-naphthalenyl]benzoic Acid

MS (m/z): 557 (M+1)

### (15) 4-[(7S)-7-[N-(tert-Butoxycarbonyl)-N-[(2R)-2-(4-chlorophenyl)-2-hydroxyethyl]amino]-5,6,7,8-tetrahydro-2-naphthalenyl]-2-fluorobenzoic Acid

MS (m/z): 541 (M+1)

### (16) 4-[(7S)-7-[N-(tert-Butoxycarbonyl)-N-[(2R)-2-(4-chlorophenyl)-2-hydroxyethyl]amino]-5,6,7,8-tetrahydro-2-naphthalenyl]-3-fluorobenzoic Acid

MS (m/z): 540 (M+1)

### (17) 4-[(7S)-7-[N-(tert-Butoxycarbonyl)-N-[(2R)-2-(4-chlorophenyl)-2-hydroxyethyl]amino]-5,6,7,8-tetrahydro-2-naphthalenyl]-2-chlorobenzoic Acid

MS (m/z): 556 (M+1)

### (18) 4-[(7S)-7-[N-(tert-Butoxycarbonyl)-N-[(2R)-2-(4-chlorophenyl)-2-hydroxyethyl]amino]-5,6,7,8-tetrahydro-2-naphthalenyl]-3-chlorobenzoic Acid

MS (m/z): 556 (M+1)

### (19) 4-[(7S)-7-[N-(tert-Butoxycarbonyl)-N-[(2R)-2-(4-chlorophenyl)-2-hydroxyethyl]amino]-5,6,7,8-tetrahydro-2-naphthalenyl]-2-methylbenzoic Acid

MS (m/z): 536 (M+1)

### (20) 4-[(7S)-7-[N-(tert-Butoxycarbonyl)-N-[(2R)-2-hydroxy-2-(4-methylphenyl)ethyl]amino]-5,6,7,8-tetrahydro-2-naphthalenyl]benzoic Acid

MS (m/z): 502 (M+1)

### (21) (2E)-3-[4-[(7S)-7-[N-(tert-Butoxycarbonyl)-N-[(2R)-2-(4-chlorophenyl)-2-hydroxyethyl]amino]-5,6,7,8-tetrahydro-2-naphthalenyl]phenyl]-2-propenoic Acid

MS (m/z): 548 (M+1)

### EXAMPLE 42

To a solution of methyl 5-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]-2-[[tert-butyl(dimethyl)silyl]oxy]benzoate (150 mg) in tetrahydrofuran (1.5 ml) was added 1M tetrabutylammonium fluoride in tetrahydrofuran (0.22 ml) at 4° C. The mixture was stirred at room temperature for 1.5 hours. The mixture was extracted with ethyl acetate and washed with water and brine. The extract was dried over magnesium sulfate and concentrated under reduced pressure. The residue was purified by column chromatography on silica gel with ethyl acetate and hexane (1:3 to 1:1) to give methyl 5-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]-2-hydroxybenzoate (123 mg) as a white solid.

(+)ESI-MS (m/z): 590 (M+Na)⁺

### EXAMPLE 43

The following compound was obtained according to a similar manner to that of Example 42.

### Methyl 3-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]-5-hydroxybenzoate

(+)ESI-MS (m/z): 590 (M+Na)⁺

### EXAMPLE 44

To a solution of 5-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]-2-hydroxybenzoate (123 mg) in methanol (1.2 ml) was added 1N sodium hydroxide (0.434 ml) and the solution was stirred at 60° C. for 1 hour. The solution was cooled to room temperature and to the solution was added 1N hydrochloric acid (0.45 ml) dropwise. The solution was extracted with ethyl acetate and washed with 1N hydrochloric acid and water. The extract was dried over magnesium sulfate, filtrated and concentrated under reduced pressure to give the carboxylic acid as a white solid. The carboxylic acid was dissolved in 1,4-dioxane (0.5 ml) and to the solution was added 4N hydrogen chloride in 1,4-dioxane (2 ml) dropwise. The solution was stirred at room temperature for 3 hours. The solution was concentrated under reduced pressure to give 5-[[(7S)-7-[[(2R)-2-(3-chlorophenyl)-2-hydroxyethyl]amino]-5,6,7,8-tetrahydro-2-naphthalenyl]oxy]-2-hydroxybenzoic acid hydrochloride (99.0 mg) as a white solid.

NMR (DMSO-d₆, δ): 1.23 (1H, m), 1.81-1.87 (2H, m), 2.27 (1H, m), 2.84 (2H, m), 3.16-3.68 (3H, m), 4.96-5.06 (1H, m), 6.30-6.38 (1H, m), 6.68-7.50 (10H, m), 8.91 (1H, br s), 9.29 (1H, br s), 12.88 (1H, br s)

(−)ESI-MS (m/z): 452 (M−HCl−1)⁻

### EXAMPLE 45

The following compounds were obtained according to a similar manner to that of Example 44.

### (1) 3-[[(7S)-7-[[(2R)-2-(3-Chlorophenyl)-2-hydroxyethyl]-amino]-5,6,7,8-tetrahydro-2-naphthalenyl]oxy]-5-methoxybenzoic Acid Hydrochloride

NMR (DMSO-d₆, δ): 1.15-1.25 (1H, m), 1.83-1.88 (2H, m), 2.27-2.32 (1H, m), 2.78-2.86 (2H, m), 3.08-3.48 (2H, m), 3.68-3.73 (1H, m), 3.80 (3H, s), 5.02-5.05 (1H, m), 6.35-6.37 (1H, m), 6.82-7.50 (10H, m), 8.91 (1H, br s), 9.32 (1H, br s)

(−)ESI-MS (m/z): 466 (M−HCl−1)⁻

### (2) 3-[[(7S)-7-[[(2R)-2-(3-Chlorophenyl)-2-hydroxyethyl]-amino]-5,6,7,8-tetrahydro-2-naphthalenyl]oxy]-5-nitrobenzoic Acid Hydrochloride

NMR (DMSO-d₆, δ): 1.23 (1H, m), 1.84-1.91 (2H, m), 2.25-2.35 (1H, m), 2.82-3.48 (4H, m), 3.68-3.79 (1H, m), 5.00-5.04 (1H, m), 6.89-6.99 (2H, m), 7.18-7.50 (5H, m), 7.70-7.87 (2H, m), 8.32-8.34 (1H, m), 10.0 (1H, br s)

(−)ESI-MS (m/z): 481 (M−HCl−1)⁻

### (3) 3-Amino-5-[[(7S)-7-[[(2R)-2-(3-chlorophenyl)-2-hydroxyethyl]amino]-5,6,7,8-tetrahydro-2-naphthalenyl]oxy]benzoic acid dihydrochloride

NMR (DMSO-d₆, δ): 0.83-0.89 (1H, m), 1.45-1.51 (1H, m), 1.84-1.91 (1H, m), 2.29-2.35 (1H, m), 2.80-2.93 (2H, m), 3.13-3.89 (3H, m), 5.03-5.07 (1H, m), 6.60-6.61 (1H, m), 6.76-7.50 (13H, m), 8.94 (1H, br s), 9.33 (1H, br s)

(+)ESI-MS (m/z): 453 (M−2HCl+1)⁺

### (4) 5-[[(7S)-7-[[(2R)-2-(3-Chlorophenyl)-2-hydroxyethyl]-amino]-5,6,7,8-tetrahydro-2-naphthalenyl]oxy]-2-cyanobenzoic Acid Hydrochloride

NMR (DMSO-d₆, δ): 1.02-1.35 (1H, m), 1.81-1.98 (2H, m), 2.15-2.25 (1H, m), 2.73-2.89 (2H, m), 3.09-3.64 (2H, m), 3.67-3.77 (1H, m), 5.00-5.04 (1H, m), 6.33 (1H, br), 6.82-7.85 (10H, m), 9.53 (1H, br s)

(+)ESI-MS (m/z): 530 (M−HCl+1)⁺

### (5) 5-[[(7S)-7-[[(2R)-2-(3-Chlorophenyl)-2-hydroxyethyl]-amino]-5,6,7,8-tetrahydro-2-naphthalenyl]oxy]-2-methylbenzoic Acid Hydrochloride

NMR (DMSO-d₆, δ): 1.71-1.90 (1H, m), 2.14-2.21 (1H, m), 2.46 (3H, s), 2.65-3.50 (7H, m), 4.88-4.93 (1H, m), 6.72-7.47 (10H, m)

(−)ESI-MS (m/z): 450 (M−HCl−1)⁻

### (6) 2-Amino-5-[[(7S)-7-[[(2R)-2-(3-Chlorophenyl)-2-hydroxyethyl]amino]-5,6,7,8-tetrahydro-2-naphthalenyl]oxy]benzoic Acid Dihydrochloride

NMR (DMSO-d₆, δ): 1.19-1.23 (1H, m), 1.50-1.53 (1H, m), 1.73-1.76 (1H, m), 2.25-2.32 (1H, m), 2.68-2.88 (2H, m), 3.10-3.28 (2H, m), 3.42-3.50 (1H, m), 5.03-5.06 (1H, m), 6.62-6.63 (1H, m), 6.73-6.75 (2H, m), 6.87 (1H, d, %=8 Hz), 7.04-7.08 (3H, m), 7.28-7.29 (1H, m), 7.38-7.43 (4H, m), 7.50 (1H, s), 8.89 (1H, br s), 9.35 (1H, br s)

(+)ESI-MS (m/z): 453 (M−2HCl+1)⁺

### (7) 3-[[(7S)-7-[[(2R)-2-(3-Chlorophenyl)-2-hydroxyethyl]-amino]-5,6,7,8-tetrahydro-2-naphthalenyl]oxy]-5-(dimethylamino)benzoic Acid Dihydrochloride

NMR (DMSO-d₆, δ): 1.51-1.55 (1H, m), 1.75-1.90 (2H, m), 2.28-2.33 (1H, m), 2.73-2.85 (2H, m), 2.93 (6H, s), 3.14-3.27 (2H, m), 3.38-3.50 (1H, m), 5.02-5.06 (1H, m), 6.63-6.64 (1H, m), 6.77-7.50 (10H, m), 8.90 (1H, br s), 9.26 (1H, br s)

(−)ESI-MS (m/z): 479 (M−2HCl−1)⁻

### (8) 3-(Acetylamino)-5-[[(7S)-7-([(2R)-2-(3-chlorophenyl)-2-hydroxyethyl]amino]-5,6,7,8-tetrahydro-2-naphthalenyl]-oxy]benzoic Acid Hydrochloride

NMR (DMSO-d₆, δ): 1.45-1.65 (1H, m), 1.74-1.91 (2H, m), 2.03 (3H, s), 2.28-2.33 (1H, m), 2.78-2.93 (2H, m), 3.10-3.64 (3H, m), 4.97-5.02 (1H, m), 6.33-6.36 (1H, m), 6.88-7.88 (10H, m), 8.95 (2H, br), 10.21 (1H, s), 13.06 (1H, br s)

(−)ESI-MS (m/z): 493 (M−HCl−1)⁻

### (9) 3-[[(7S)-7-[[(2R)-2-(3-Chlorophenyl)-2-hydroxyethyl]-amino]-5,6,7,8-tetrahydro-2-naphthalenyl]oxy]-5-hydroxybenzoic Acid Hydrochloride

NMR (DMSO-d₆, δ): 1.42-1.60 (1H, m), 1.71-1.82 (2H, m), 2.22-2.35 (1H, m), 2.77-2.94 (2H, m), 3.07-3.75 (3H, m), 5.01-5.05 (1H, m), 6.34 (1H, br), 6.59-7.50 (10H, m), 9.03 (2H, br), 10.01 (1H, s), 12.94 (1H, br s)

(−)ESI-MS (m/z): 452 (M−HCl−1)⁻

### (10) 5-[[(7S)-7-[[(2R)-2-(3-Chlorophenyl)-2-hydroxyethyl]-amino]-5,6,7,8-tetrahydro-2-naphthalenyl]oxy]-2-(methylamino)benzoic Acid Dihydrochloride

NMR (DMSO-d₆, δ): 1.46-1.61 (1H, m), 1.67-1.90 (2H, m), 2.24-2.36 (1H, m), 2.73-2.89 (2H, m), 2.88 (3H, s), 3.14-3.23 (3H, m), 5.03-5.08 (2H, m), 6.60-6.77 (3H, m), 7.05-7.21 (2H, m), 7.35-7.49 (5H, m), 8.32 (1H, s), 8.93 (1H, br s), 9.39 (1H, br s)

(−)ESI-MS (m/z): 465 (M−2HCl−1)⁻

### (11) 2-(Acetylamino)5-[[(7S)-7-[[(2R)-2-(3-chlorophenyl)-2-hydroxyethyl]amino]-5,6,7,8-tetrahydro-2-naphthalenyl]-oxy]benzoic Acid Hydrochloride

NMR (DMSO-d₆, δ): 1.59-1.83 (3H, m), 2.11 (3H, s), 2.25-2.39 (1H, m), 2.75-2.86 (2H, m), 2.92-3.40 (2H, m), 3.55-3.63 (1H, m), 5.03-5.08 (1H, m), 6.35 (1H, br), 6.74-7.50 (9H, m), 8.38 (1H, d, J=9 Hz), 8.94 (1H, br s), 9.36 (1H, br s), 10.84 (1H, s), 13.30 (1H, br)

(−)ESI-MS (m/z): 493 (M−HCl−1)⁻

### (12) 5-[[(7S)-7-[[(2R)-2-(3-Chlorophenyl)-2-hydroxyethyl]-amino]-5,6,7,8-tetrahydro-2-naphthalenyl]oxy]-2-[(methylsulfonyl)amino]benzoic Acid Hydrochloride

NMR (DMSO-d₆, δ): 1.46-1.65 (1H, m), 1.66-1.94 (2H, m), 2.27-2.41 (1H, m), 2.76-2.94 (2H, m), 3.15 (3H, S), 3.15-3.77 (3H, m), 5.05-5.10 (1H, m), 6.36 (1H, br), 6.78-6.88 (2H, m), 7.08-7.61 (8H, m), 8.98 (1H, br s), 9.47 (1H, br s), 10.43 (1H, br s)

(−)ESI-MS (m/z): 529 (M−HCl−1)⁻

### (13) 5-[[(7S)-7-[[(2R)-2-(3-Chlorophenyl)-2-hydroxyethyl]-amino]-5,6,7,8-tetrahydro-2-naphthalenyl]oxy]-2-[(ethoxycarbonyl)amino]benzoic Acid Hydrochloride

NMR (DMSO-d₆, δ): 1.24 (2H, t, J=7 Hz), 1.49-1.67 (1H, m), 1.67-1.89 (2H, m), 2.28-2.40 (1H, m), 2.78-2.92 (2H, m), 3.21-3.81 (3H, m), 4.18 (3H, q, J=7 Hz), 5.04-5.08 (1H, m), 6.36 (1H, br s), 6.75-6.85 (2H, m), 7.13 (1H, d, J=8 Hz), 7.29-7.50 (6H, m), 8.25 (1H, d, J=9 Hz), 8.93 (1H, br s), 9.39 (1H, br s), 10.50 (1H, br s)

(+)ESI-MS (m/z): 529 (M−HCl+1)⁺

### (14) 2-[N-Acetyl-N-methylamino]-5-[[(7S)-7-[[(2R)-2-(3-chlorophenyl)-2-hydroxyethyl]amino]-5,6,7,8-tetrahydro-2-naphthalenyl]oxy]benzoic Acid Hydrochloride

NMR (DMSO-d₆, δ): 1.22-1.40 (1H, m), 1.63 (3H, s), 1.75-2.02 (2H, m), 2.27-2.40 (1H, m), 2.81-2.95 (2H, m), 3.02 (3H, s), 3.16-3.56 (3H, m), 5.03-5.07 (1H, m), 6.35 (1H, br s), 6.90-7.50 (10H, m), 8.95-9.32 (2H, br), 13.20 (1H, br s)

(−)ESI-MS (m/z): 507 (M−HCl−1)⁻

### (15) 2-(Benzoylamino)-5-[[(7S)-7-[[(2R)-2-(3-chlorophenyl)-2-hydroxyethyl]amino]-5,6,7,8-tetrahydro-2-naphthalenyl]oxy]benzoic Acid Hydrochloride

NMR (DMSO-d₆, δ): 1.85-1.91 (2H, m), 2.32-2.40 (1H, m), 2.75-3.56 (6H, m), 5.08-5.13 (1H, m), 6.37 (1H, br s), 6.51-6.58 (2H, m), 7.15 (1H, d, J=8 Hz), 7.34-7.65 (9H, m), 7.93-7.97 (2H, m), 8.69 (1H, d, J=9 Hz), 8.99 (1H, br s), 9.56 (1H, br s), 11.99 (1H, s)

(−)ESI-MS (m/z): 555 (M−HCl−1)⁻

### (16) 3-(Benzoylamino)-5-[[(7S)-7-[[(2R)-2-(3-chlorophenyl)-2-hydroxyethyl]amino]-5,6,7,8-tetrahydro-2-naphthalenyl]oxy]benzoic Acid Hydrochloride

NMR (DMSO-d₆, δ): 1.20-1.33 (1H, m), 1.79-1.91 (2H, m), 2.23-2.38 (1H, m), 2.76-2.94 (2H, m), 3.15-3.69 (3H, m), 4.99-5.06 (1H, m), 6.34-6.36 (1H, m), 6.85-8.18 (15H, m), 8.96 (2H, br), 10.50 (1H, s), 13.08 (1H, br)

(−)ESI-MS (m/z): 555 (M−HCl−1)⁻

### (17) 3-[[(7S)-7-[[(2R)-2-(3-Chlorophenyl)-2-hydroxyethyl]-amino]-5,6,7,8-tetrahydro-2-naphthalenyl]oxy]-5-(2-furoylamino)benzoic Acid Hydrochloride

NMR (DMSO-d₆, δ): 1.17-1.32 (1H, m), 1.77-1.94 (2H, m), 2.23-2.35 (1H, m), 2.80-2.94 (2H, m), 3.13-3.69 (3H, m), 4.99-5.06 (1H, m), 6.35-6.36 (1H, m), 6.70 (1H, dd, 1.5, 3.4 Hz), 6.85-6.93 (2H, m), 7.12-7.21 (2H, m), 7.39-7.50 (5H, m), 7.78-7.80 (1H, m), 7.94-7.95 (1H, m), 8.14-8.15 (1H, m), 8.91 (1H, br s), 9.25 (1H, br s), 10.45 (1H, s), 13.10 (1H, br s)

(−)ESI-MS (m/z): 555 (M−HCl−1)⁻

### (18) 3-[[(7S)-7-[[(2R)-2-(3-Chlorophenyl)-2-hydroxyethyl]-amino]-5,6,7,8-tetrahydro-2-naphthalenyl]oxy]-5-[(2,2-dimethylpropanoyl)amino]benzoic Acid Hydrochloride

NMR (DMSO-d₆, δ): 1.20 (9H, s), 1.77-1.94 (2H, m), 2.24-2.38 (1H, m), 2.77-3.70 (6H, m), 5.00-5.06 (1H, m), 6.35 (1H, br s), 6.82-7.43 (7H, m), 7.50-7.51 (1H, m), 7.73-7.74 (1H, m), 8.04-8.05 (1H, m), 8.89-9.21 (2H, br), 9.48 (1H, s), 12.95 (1H, br s)

(−)ESI-MS (m/z): 535 (M−HCl−1)⁻

### (19) 3-[[(7S)-7-[[(2R)-2-(3-Chlorophenyl)-2-hydroxyethyl]-amino]-5,6,7,8-tetrahydro-2-naphthalenyl]oxy]-5-[(methoxycarbonyl)amino]benzoic Acid Hydrochloride

NMR (DMSO-d₆, δ): 1.13-1.26 (1H, m), 1.76-1.95 (2H, m), 2.25-2.38 (1H, m), 2.80-2.93 (2H, m), 3.14-3.49 (3H, m), 3.69 (3H, s), 5.03-5.08 (1H, m), 6.35-6.37 (1H, m), 6.81-7.50 (9H, m), 7.79 (1H, d, J=1.3 Hz), 8.91 (1H, br s), 9.35 (1H, br s), 9.95 (1H, s), 13.03 (1H, br s)

(−)ESI-MS (m/z): 509 (M−HCl−1)⁻

### (20) 3-[[(Benzyloxy)carbonyl]amino]-5-[[(7S)-7-[[(2R)-2-(3-chlorophenyl)-2-hydroxyethyl]amino]-5,6,7,8-tetrahydro-2-naphthalenyl]oxy]benzoic Acid Hydrochloride

NMR (DMSO-d₆, δ): 1.12-1.34 (1H, m), 1.84-1.91 (2H, m), 2.28-2.38 (1H, m), 2.80-2.93 (2H, m), 3.13-3.59 (3H, m), 4.49 (1H, s), 5.04-5.08 (1H, m), 5.15 (1H, s), 6.35 (1H, br), 6.82-7.83 (15H, m), 8.94 (1H, br s), 9.35 (1H, br s), 9.95 (1H, s), 13.05 (1H, br s)

(−)ESI-MS (m/z): 586 (M−HCl−1)⁻

### (21) 5-[[(7S)-7-[[(2R)-2-(3-Chlorophenyl)-2-hydroxyethyl]-amino]-5,6,7,8-tetrahydro-2-naphthalenyl]oxy]-2-[(2,2-dimethylpropanoyl)amino]benzoic Acid Hydrochloride

NMR (DMSO-d₆, δ): 1.24 (9H, s), 1.83-1.91 (1H, m), 2.28-2.35 (1H, m), 2.78-2.92 (3H, m), 3.20-3.52 (3H, m), 3.56-3.78 (1H, m), 5.05-5.09 (1H, m), 6.36 (1H, br), 6.37 (1H, d, J=2 Hz), 6.82 (1H, dd, J=2, 8 Hz), 7.13 (1H, d, J=8 Hz), 7.30 (1H, dd, J=3, 9 Hz), 7.34-7.52 (6H, m), 8.61 (1H, d, J=9 Hz), 8.95 (1H, br s), 9.43 (1H, br s), 11.33 (1H, s)

(−)ESI-MS (m/z): 535 (M−HCl−1)⁻

### (22) 5-[[(7S)-7-[[(2R)-2-(3-Chlorophenyl)-2-hydroxyethyl]amino]-5,6,7,8-tetrahydro-2-naphthalenyl]oxy]-2-(2-oxo-1-pyrrolidinyl)benzoic Acid Hydrochloride

NMR (DMSO-d₆, δ): 1.75-1.93 (1H, m), 2.03-2.16 (2H, m), 2.25-2.37 (2H, m), 2.79-3.78 (10H, m), 5.04-5.08 (1H, m), 6.35 (1H, br), 6.84-7.62 (10H, m), 8.95 (1H, br s), 9.39 (1H, br s), 12.91 (1H, br s)

(−)ESI-MS (m/z): 519 (M−HCl−1)⁻

### (23) 3-[(Anilinocarbonyl)amino]-5-[[(7S)-7-[[(2R)-2-(3-chlorophenyl)-2-hydroxyethyl]amino]-5,6,7,8-tetrahydro-2-naphthalenyl]oxy]benzoic Acid Hydrochloride

NMR (DMSO-d₆, δ): 1.20-1.31 (1H, m), 1.75-1.96 (2H, m), 2.22-2.35 (1H, m), 2.78-2.98 (4H, m), 3.61-3.74 (1H, m), 5.00-5.04 (1H, m), 6.34-6.36 (1H, m), 6.84-7.00 (4H, m), 7.15-7.50 (10H, m), 8.32-8.33 (1H, m), 8.99 (2H, br), 9.12 (1H, s), 9.48 (1H, s), 12.98 (1H, br s)

(−)ESI-MS (m/z): 570 (M−HCl−1)⁻

### (24) 3-[[(7S)-7-[[(2R)-2-(3-Chlorophenyl)-2-hydroxyethyl]-amino]-5,6,7,8-tetrahydro-2-naphthalenyl]oxy]-5-[[(methylamino)carbonyl]amino]benzoic Acid Hydrochloride

NMR (DMSO-d₆, δ): 1.13-1.23 (1H, m), 1.84-1.98 (2H, m), 2.28-2.36 (1H, m), 2.61 (3H, d, J=4 Hz), 2.79-2.99 (4H, m), 3.56-3.70 (1H, m), 5.00-5.05 (1H, m), 6.19-6.21 (1H, m), 6.34-6.35 (1H, m), 6.80-7.00 (3H, m), 7.16 (1H, d, J=8 Hz), 7.41-7.65 (5H, m), 7.88 (1H, s), 8.99 (1H, br s), 9.08 (1H, s), 9.19 (1H, br s), 12.93 (1H, br)

(+)ESI-MS (m/z): 510 (M−HCl+1)⁺

### (25) 4-[(7S)-7-[[(2R)-2-(3-Chlorophenyl)-2-hydroxyethyl]-amino]-5,6,7,8-tetrahydro-2-naphthalenyl]-3-methylbenzoic Acid Hydrochloride

NMR (200 MHz, DMSO-d₆, δ): 1.70-1.98 (2H, m), 2.20-2.36 (1H, m), 2.28 (3H, s), 2.71-2.99 (3H, m), 3.11-3.32 (2H, m), 3.52 (1H, br), 5.00 (1H, br), 6.33-6.38 (1H, m), 6.48-6.58 (1H, m), 6.87-6.91 (1H, m), 7.11-7.52 (7H, m), 7.78-7.86 (1H, m), 8.87 (1H, br), 12.9 (1H, br)

(−)ESI-MS (m/z): 434 (M−HCl−1)⁻

### (26) 2-[[(7S)-7-[[(2R)-2-(3-Chlorophenyl)-2-hydroxyethyl]-amino]-5,6,7,8-tetrahydro-2-naphthalenyl]oxy]-5-chloroisonicotinic Acid Hydrochloride

NMR (200 MHz, DMSO-d₆, δ): 1.71-1.91 (2H, m), 2.27 (1H, br), 2.81-2.94 (3H, m), 3.12-3.64 (3H, m), 5.00-5.05 (1H, m), 6.36 (1H, br), 6.29 (1H, s), 6.92-6.98 (1H, m), 7.17 (1H, d, J=8.1 Hz), 7.11-7.52 (7H, m), 7.29 (1H, s), 7.36-7.47 (3H, m), 7.51 (1H, s), 8.29 (1H, s), 8.89 (1H, br), 9.18 (1H, br)

(−)ESI-MS (m/z): 471 (M−HCl−1)⁻

### (27) 6-[(7S)-7-[[(2R)-2-(3-Chlorophenyl)-2-hydroxyethyl]-amino]-5,6,7,8-tetrahydro-2-naphthalenyl]nicotinic Acid Dihydrochloride

NMR (200 MHz, DMSO-d₆, δ): 1.74-1.99 (2H, m), 2.32-2.49 (2H, m), 2.85-3.04 (4H, m), 3.38 (1H, br), 3.52 (1H, br), 5.07 (1H, d, J=8.0 Hz), 7.28 (1H, d, J=7.9 Hz), 7.47-7.59 (4H, m), 7.94 (1H, d, J=7.8 Hz), 7.96 (1H, s), 8.07 (1H, d, J=8.3 Hz), 8.29-8.34 (1H, m), 8.97 (1H, br), 9.12 (1H, s), 9.31 (1H, br)

(−)ESI-MS (m/z): 421 (M−2HCl−1)⁻

### (28) 4-[(7S)-7-[[(2R)-2-(3-Chlorophenyl)-2-hydroxyethyl]-amino]-5,6,7,8-tetrahydro-2-naphthalenyl]-2-methoxybenzoic Acid Hydrochloride

NMR (200 MHz, DMSO-d₆, δ): 1.72-1.96 (1H, m), 2.25-2.40 (1H, m), 2.83-3.19 (5H, m), 3.40-3.42 (1H, m), 3.54 (1H, br), 3.90 (3H, s), 5.04-5.08 (1H, m), 6.38 (1H, br), 7.21-7.29 (3H, m), 7.40-7.45 (3H, m), 7.51-7.55 (3H, m), 7.72 (1H, d, J=7.9 Hz), 9.18 (1H, br)

(−)ESI-MS (m/z): 450 (M−HCl−1)⁻

### (29) 4-[(7S)-7-[[(2R)-2-(3-Chlorophenyl)-2-hydroxyethyl]-amino]-5,6,7,8-tetrahydro-2-naphthalenyl]-2-fluorobenzoic Acid Hydrochloride

NMR (200 MHz, DMSO-d₆, δ): 1.85-1.98 (1H, m), 2.31-2.36 (1H, m), 2.83-3.18 (5H, m), 3.35-3.42 (1H, m), 3.53 (1H, br), 3.90 (3H, s), 5.03-5.08 (1H, m), 6.93 (1H, dd, J=8.0 Hz), 9.17 (1H, br)

(−)ESI-MS (m/z): 439 (M−HCl−1)⁻

### (30) 4-[(7S)-7-[[(2R)-2-(3-Chlorophenyl)-2-hydroxyethyl]-amino]-5,6,7,8-tetrahydro-2-naphthalenyl]-3-methoxybenzoic Acid Hydrochloride

NMR (200 MHz, DMSO-d₆, δ): 1.71-1.98 (2H, m), 2.32 (1H, br), 2.28 (3H, s), 2.70-3.01 (3H, m), 3.11-3.30 (2H, m), 3.54-3.63 (1H, m), 3.81 (3H, s), 5.05-5.10 (1H, m), 6.37 (1H, br), 7.14-7.63 (10H, m), 9.14 (1H, br)

(−)ESI-MS (m/z): 450 (M−HCl−1)⁻

### EXAMPLE 46

To a solution of methyl 3-[[(7S)-7-[N-(tert-butoxycarbonyl)-N-[(2R)-2-(3-chlorophenyl)-2-hydroxyethyl]-amino]-5,6,7,8-tetrahydro-2-naphthalenyl]oxy)-5-nitrobenzoate (150 mg) in a mixed solution of ethanol (1.5 ml) and water (0.5 ml) were added iron powder (42.1 mg) and ammonium chloride (6.72 mg). The solution was heated under reflux for 2 hours. After cooling to room temperature, the precipitate was filtered through a pad of Celite®. After concentration under reduced pressure, the residue was extracted with ethyl acetate and successively washed with saturated aqueous sodium bicarbonate and brine, and dried over magnesium sulfate. After concentration under reduced pressure, the residue was purified by column chromatography on silica gel with ethyl acetate and hexane (1:3) to give methyl 3-amino-5-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]benzoate (132 mg) as a white solid.

(+)ESI-MS (m/z): 589 (M+Na)⁺

### EXAMPLE 47

To a solution of tert-butyl N-[(2R)-2-(3-chlorophenyl)-2-hydroxyethyl]-N-[(2S)-7-(4-fluoro-3-formylphenoxy)-1,2,3,4-tetrahydro-2-naphthalenyl]carbamate (100 mg) in dimethyl sulfoxide (1 ml) were added phenol (19.5 μl) and potassium carbonate (76.8 mg) and the mixture was stirred at 100° C. for 1.5 hours. The solution was diluted with water and ethyl acetate. The organic layer was separated and washed with brine. The extract was dried over magnesium sulfate, filtrated and concentrated under reduced pressure. The residue was purified by column chromatography on silica gel with ethyl acetate and hexane to give tert-butyl N-[(2R)-2-(3-chlorophenyl)-2-hydroxyethyl]-N-[(2S)-7-(3-formyl-4-phenoxyphenoxy)-1,2,3,4-tetrahydro-2-naphthalenyl]-carbamate (70.1 mg) as a white solid.

(+)ESI-MS (m/z): 636 (M+Na)⁺

### EXAMPLE 48

The following compound was obtained according to a similar manner to that of Example 47.

### Ethyl 2-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]-5-chloroisonicotinate

(+)ESI-MS (m/z): 623 (M+Na)⁺

### EXAMPLE 49

To a solution of methyl 3-amino-5-[[(7S)-7-[N-(tert-butoxycarbonyl)-N-[(2R)-2-(3-chlorophenyl)-2-hydroxyethyl]-amino]-5,6,7,8-tetrahydro-2-naphthalenyl]oxy]benzoate (80 mg) in acetonitrile (1 ml) were added sodium cyanoborohydride (26.6 mg), acetic acid (0.02 ml) and 35% formaldehyde solution (0.328 ml). The solution was stirred at room temperature for 17 hours. The solution was concentrated under reduced pressure. The residue was extracted with ethyl acetate and washed with saturated aqueous sodium bicarbonate and water. The extract was dried over magnesium sulfate, filtrated and concentrated under reduced pressure. The residue was purified by column chromatography on silica gel with ethyl acetate and hexane to give methyl 3-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]-5-(dimethylamino)benzoate (70.5 mg) as a white solid.

(+)ESI-MS (m/z): 617 (M+Na)⁺

### EXAMPLE 50

To a solution of methyl 3-amino-5-[[(7S)-7-[N-(tert-butoxycarbonyl)-N-[(2R)-2-(3-chlorophenyl)-2-hydroxyethyl]-amino]-5,6,7,8-tetrahydro-2-naphthalenyl]oxy]benzoate (73 mg) and pyridine (0.021 ml) in dichloromethane (0.1 ml) was added acetic anhydride (0.0013 ml) dropwise at 4° C. The solution was stirred at room temperature for 2 hours. To the solution was added water and the solution was extracted with ethyl acetate and washed with water and brine. The extract was dried over magnesium sulfate, filtrated and concentrated under reduced pressure. The residue was purified by column chromatography on silica gel with ethyl acetate and hexane to give methyl 3-(acetylamino)-5-[[(7S)-7-[N-(tert-butoxycarbonyl)-N-[(2R)-2-(3-chlorophenyl)-2-hydroxyethyl]amino]-5,6,7,8-tetrahydro-2-naphthalenyl)oxy]-benzoate (75 mg) as a white solid.

(−)ESI-MS (m/z): 607 (M−1)⁻

### EXAMPLE 51

To a solution of methyl 3-amino-5-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]-benzoate (110 mg) and pyridine (0.019 ml) in dichloromethane (1.1 ml) was added benzoyl chloride (0.025 ml) dropwise at 4° C. The solution was stirred at the same temperature for 30 minutes. To the solution were added water and ethyl acetate. The mixture was extracted with ethyl acetate. The extract was washed with water and brine, dried over magnesium sulfate and evaporated. The residue was purified by column chromatography on silica gel with ethyl acetate and hexane (1:3) to give methyl 3-(benzoylamino)-5-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]-oxy]benzoate (129 mg) as a white solid.

(+)ESI-MS (m/z): 693 (M+Na)⁺

### EXAMPLE 52

The following compounds were obtained according to a similar manner to that of Example 51.

### (1) Methyl 3-[[(7S)-7-[N-(tert-butoxycarbonyl)-N-[(2R)-2-(3-chlorophenyl)-2-hydroxylethyl]amino]-5,6,7,8-tetrahydro-2-naphthalenyl]oxy]-5-(2-furoylamino)benzoate

(+)ESI-MS (m/z): 683 (M+Na)⁺

### (2) Methyl 3-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]-5-[(2,2-dimethylpropanoyl)amino]benzoate

(+)ESI-MS (m/z): 673 (M+Na)⁺

### EXAMPLE 53

To a solution of methyl 3-amino-5-[[(7S)-7-[N-(tert-butoxycarbonyl)-N-[(2R)-2-(3-chlorophenyl)-2-hydroxyethyl]-amino]-5,6,7,8-tetrahydro-2-naphthalenyl]oxy]benzoate (110 mg) in a mixed solution of tetrahydrofuran (1 ml) and water (1 ml) was added two drops of 1N sodium hydroxide. To the solution was added methyl chloroformate (0.018 ml) dropwise at 4° C. and the reaction mixture was stirred at the same temperature for 30 minutes. To the solution was added water and the mixture was extracted with ethyl acetate and washed with water and brine. The extract was dried over magnesium sulfate, filtrated and concentrated under reduced pressure. The residue was purified by column chromatography on silica gel with ethyl acetate and hexane (1:2 to 1:1) to give methyl 3-[[(7S)-7-[N-(tert-butoxycarbonyl)-N-[(2R)-2-(3-chlorophenyl)-2-hydroxyethyl]amino)-5,6,7,8-tetrahydro-2-naphthalenyl]oxy]-5-[(methoxycarbonyl)amino]benzoate (120 mg) as a white solid.

(+)ESI-MS (m/z): 647 (M+Na)⁺

### EXAMPLE 54

To a solution of methyl 3-amino-5-[[(7S)-7-[N-(tert-butoxycarbonyl)-N-[(2R)-2-(3-chlorophenyl)-2-hydroxyethyl]-amino]-5,6,7,8-tetrahydro-2-naphthalenyl]oxy]benzoate (100 mg) in a mixed solution of acetone (0.66 ml) and water (0.33 ml) was added sodium carbonate (37.4 mg). To the solution was added benzyl chloroformate (0.03 ml) dropwise at 4° C. and the reaction mixture was stirred at room temperature for 2 hours. To the solution was added water and the mixture was extracted with ethyl acetate and washed with water and brine. The extract was dried over magnesium sulfate, filtrated and concentrated under reduced pressure. The residue was purified by column chromatography on silica gel with ethyl acetate and hexane (1:2 to 1:1) to give methyl 3-[[(benzyloxy)carbonyl]amino]-5-[](7S)-7-[N-(tert-butoxycarbonyl)-N-[(2R)-2-(3-chlorophenyl)-2-hydroxyethyl]-amino]-5,6,7,8-tetrahydro-2-naphthalenyl]oxy]benzoate (113 mg) as a white solid.

(+)ESI-MS (m/z): 723 (M+Na)⁺

### EXAMPLE 55

To a solution of methyl 3-amino-5-[[(7S)-7-[N-(tert-butoxycarbonyl)-N-[(2R)-2-(3-chlorophenyl)-2-hydroxyethyl]-amino]-5,6,7,8-tetrahydro-2-naphthalenyl]oxy]benzoate (110 mg) in dichloromethane (1 ml) was added methyl isocyanate (33.2 mg) and the solution was stirred at room temperature for 2 hours. To the solution was added N,N-diisopropylethylamine (6.8 μl) and the solution was stirred at room temperature for 1 hour. To the solution was added 28% ammonia solution and then added ethyl acetate. The mixture was extracted with ethyl acetate and washed with brine. The extract was dried over magnesium sulfate, filtrated and concentrated under reduced pressure. The residue was purified by column chromatography on silica gel with ethyl acetate and hexane (1:2 to 1:1) to give methyl 3-[[(7S)-7-[N-(tert-butoxycarbonyl)-N-[(2R)-2-(3-chlorophenyl)-2-hydroxyethyl]amino]-5,6,7,8-tetrahydro-2-naphthalenyl]oxy]-5-[[(methylamino)carbonyl]amino]benzoate (95.2 mg) as a white solid.

(+)ESI-MS (m/z): 646 (M+Na)⁺

### EXAMPLE 56

The following compound was obtained according to a similar manner to that of Example 55.

### Methyl 3-[(anilinocarbonyl)amino]-5-[[(7S)-7-[N-(tert-butoxycarbonyl)-N-[(2R)-2-(3-chlorophenyl)-2-hydroxyethyl]-amino]-5,6,7,8-tetrahydro-2-naphthalenyl]oxy]benzoate

(+)ESI-MS (m/z): 708 (M+Na)⁺

### EXAMPLE 57

To a solution of methyl 5-[[(7S)-7-amino-5,6,7,8-tetrahydro-2-naphthalenyl]oxy]-2-(1-pyrrolidinyl)benzoate (232 mg) in ethanol (5 ml) was added (2R)-2-(3-chlorophenyl)oxirane (97.9 mg) and the mixture was refluxed for 18 hours. After cooling to room temperature, the solvent was removed by evaporation and the residue was purified by column chromatography on silica gel with chloroform and methanol (100:0 to 90:10) to give methyl 5-[[(7S)-7-[[(2R)-2-(3-chlorophenyl)-2-hydroxyethyl]amino]-5,6,7,8-tetrahydro-2-naphthalenyl]oxy]-2-(1-pyrrolidinyl)benzoate (173 mg) as a white solid.

(+)ESI-MS (m/z): 521 (M+1)⁺

### EXAMPLE 58

To a solution of methyl 5-[[(7S)-7-[[(2R)-2-(3-chlorophenyl)-2-hydroxyethyl]amino]-5,6,7,8-tetrahydro-2-naphthalenyl]oxy]-2-(1-pyrrolidinyl)benzoate (70 mg) in ethanol (0.7 ml) was added 1N sodium hydroxide (0.336 ml) and the mixture was stirred at 75° C. for 24 hours. To the mixture was added 1N hydrochloric acid (0.202 ml) and the mixture was stirred for 15 minutes and concentrated under reduced pressure. The residue was washed with water to give sodium 5-[[(7S)-7-[[(2R)-2-(3-chlorophenyl)-2-hydroxyethyl]-10 amino]-5,6,7,8-tetrahydro-2-naphthalenyl]oxy]-2-(1-pyrrolidinyl)benzoate (51 mg) as a white solid.

NMR (DMSO-d₆, δ): 1.48-1.58 (1H, m), 1.88-2.00 (5H, m), 2.36-2.79 (6H, m), 3.10-3.22 (5H, m), 4.63-4.65 (1H, m), 5.40 (1H, br), 6.69-6.72 (2H, m), 7.04-7.16 (4H, m), 7.26-7.41 (4H, m)

(−)ESI-MS (m/z): 505 (M−Na)⁻

### EXAMPLE 59

To a solution of sodium 5-[[(7S)-7-[[(2R)-2-(3-chlorophenyl)-2-hydroxyethyl]amino]-5,6,7,8-tetrahydro-2-naphthalenyl]oxy]-2-(1-pyrrolidinyl)benzoate (51 mg) in methanol (0.5 ml) was added 1N hydrochloric acid (0.29 ml) and the solution was stirred for 10 minutes. The solvent was evaporated and the residue was washed with water to give 5-[[(7S)-7-[[(2R)-2-(3-chlorophenyl)-2-hydroxyethyl]amino]-5,6,7,8-tetrahydro-2-naphthalenyl]oxy]-2-(1-pyrrolidinyl)benzoic acid dihydrochloride (47.8 mg) as a white solid.

(−)ESI-MS (m/z): 505 (M−2HCl−1)⁻

### EXAMPLE 60

To a solution of methyl 4-[(7S)-7-[[(2R)-2-(6-chloro-3-pyridyl)-2-hydroxyethyl]amino]-5,6,7,8-tetrahydro-2-naphthalenyl]benzoate (128 mg) in methanol (5.0 ml) was added 1N sodium hydroxide (0.30 ml) and the mixture was stirred for 2 hours at room temperature. The mixture was evaporated in vacuo to give sodium 4-[(7S)-7-[[(2R)-2-(6-chloro-3-pyridyl)-2-hydroxyethyl]amino]-5,6,7,8-tetrahydro-2-naphthalenyl)benzoate (90 mg) as a colorless powder.

NMR (DMSO-d₆, δ): 1.50-1.70 (1H, m), 1.90-2.10 (1H, m), 2.50-3.50 (7H, m), 4.70-4.80 (1H, m), 7.10-7.15 (1H, m), 7.20-7.60 (5H, m), 7.70-8.00 (3H, m), 8.40 (1H, s)

MS (m/z): 423 (M+1)

### EXAMPLE 61

The following compounds were obtained according to a similar manner to that of Example 60.

### (1) Sodium 3-[[(7S)-7-[[(2R)-2-hydroxy-2-(6-methyl-3-pyridyl)ethyl]amino]-5,6,7,8-tetrahydro-2-naphthalenyl]oxy]benzoate

NMR (DMSO-d₆, δ): 1.40-1.50 (1H, m), 1.80-1.90 (1H, m), 2.43 (3H, s), 2.50-3.00 (7H, m), 4.60-4.70 (1H, m), 6.70-6.80 (2H, m), 6.90-7.70 (7H, m), 8.40 (1H, d, J=2 Hz)

MS (m/z): 419 (M+1)

### (2) Sodium 4-[(7S)-7-[[(2R)-2-hydroxy-2-(6-methyl-3-pyridyl)ethyl]amino]-5,6,7,8-tetrahydro-2-naphthalenyl]benzoate

NMR (DMSO-d₆, δ): 1.50-1.70 (1H, m), 1.95-2.10 (1H, m), 2.44 (3H, s), 2.40-3.20 (7H, m), 4.60-4.75 (1H, m), 7.10-7.60 (7H, m), 7.90 (2H, d, J=8 Hz), 8.43 (1H, d, J=2 Hz)

MS (m/z): 452 (M+1)

### (3) Sodium 4-[(7S)-7-[[(2R)-2-(4-chlorophenyl)-2-hydroxyethyl]amino)-5,6,7,8-tetrahydro-2-naphthalenyl]-3-methoxybenzoate

NMR (DMSO-d₆, δ): 1.40-1.55 (1H, m), 1.80-2.00 (1H, m), 2.70-3.30 (7H, m), 3.74 (3H, s), 4.60-4.70 (1H, m), 6.85-6.95 (2H, m), 6.90-7.60 (10H, m)

MS (m/z): 452 (M+1)

### (4) Sodium 1-[(7S)-7-[[(2R)-2-(4-chlorophenyl)-2-hydroxyethyl]amino]-5,6,7,8-tetrahydro-2-naphthalenyl)-4-piperidinecarboxylate

NMR (DMSO-d₆, δ): 1.30-3.00 (16H, m), 3.40-3.50 (2H, m), 4.60-4.70 (1H, m), 6.75-6.90 (3h, m), 7.20-7.40 (4H, m)

MS (m/z): 429 (M+1)

### EXAMPLE 62

To a solution of tert-butyl N-[(2R)-2-(4-chlorophenyl)-2-hydroxyethyl]-N-[(2S)-7-hydroxy-1,2,3,4-tetrahydro-2-naphthalenyl)carbamate (250 mg) in methyl sulfoxide (20 ml) was added methyl 2,3-dichloroisonicotinate (246 mg) and potassium carbonate (124 mg), and the mixture was stirred at 80° C. for 18 hours under nitrogen. The mixture was diluted with ethyl acetate and water. The organic layer was separated, washed with brine, dried over magnesium sulfate and evaporated. The residue was purified by column chromatography on silica gel (hexane/ethyl acetate=2/1) to give methyl 2-[[(7S)-7-[N-(tert-butoxycarbonyl)-N-[(2R)-2-(4-chlorophenyl)-2-hydroxyethyl]amino]-5,6,7,8-tetrahydro-2-naphthalenyl]oxy]-3-chloroisonicotinate (210 mg) as a colorless powder.

MS (m/z): 588 (M+1)

### EXAMPLE 63

To a solution of tert-butyl N-[(2R)-2-(4-chlorophenyl)-2-hydroxyethyl]-N-[(2S)-7-(4-formylphenyl)-1,2,3,4-tetrahydro-2-naphthalenyl]carbamate (620 mg) in tetrahydrofuran (15 ml) was added sodium hydride (64 mg) and ethyl(diethoxyphosphinyl)acetate (357 mg), and stirred at room temperature for 0.5 hour under nitrogen. The mixture was diluted with ethyl acetate and water. The organic layer was separated, washed with brine, dried over magnesium sulfate and evaporated. The residue was purified by column chromatography on silica gel (hexane/ethyl acetate=2/1) to give ethyl(2E)-3-[4-[(7S)-7-[N-(tert-butoxycarbonyl)-N-[(2R)-2-(4-chlorophenyl)-2-hydroxyethyl]amino]-5,6,7,8-tetrahydro-2-naphthalenyl]phenyl]-2-propenoate (400 mg) as a colorless powder.

MS (m/z): 576 (M+1)

### EXAMPLE 64

A mixture of ethyl(2E)-3-[4-[(7S)-7-[N-(tert-butoxycarbonyl)-N-[(2R)-2-(4-chlorophenyl)-2-hydroxyethyl]amino]-5,6,7,8-tetrahydro-2-naphthalenyl]phenyl]-2-propenoate (140 mg), 10% palladium on activated carbon (50% wet, 50 mg), ethanol (10 ml) and chlorobenzene (10 ml) was stirred at room temperature in the presence of hydrogen at an atmospheric pressure for 1.2 hours. To the reaction mixture was added ethanol to dissolve the precipitates. After removal of 10% palladium on activated carbon by filtration, the filtrate was evaporated under reduced pressure. The residue was added 4N hydrogen chloride in 1,4-dioxane (4 ml) dropwise. The solution was stirred at room temperature for 3 hours. The solution was dissolved into a mixture of saturated aqueous sodium bicarbonate and ethyl acetate. After separation, the organic layer was washed with brine, dried over magnesium sulfate and evaporated under reduced pressure. The residue was added 1N sodium hydroxide (0.30 ml) and the mixture was stirred for 2 hours at room temperature. The mixture was evaporated in vacuo to give sodium 3-[4-[(7S)-7-[[(2R)-2-(4-chlorophenyl)-2-hydroxyethyl]amino]-5,6,7,8-tetrahydro-2-naphthalenyl]phenyl]propanoate (50 mg) as a colorless powder.

NMR (DMSO-d₆, δ): 1.40-1.55 (1H, m), 1.90-2.00 (1H, m), 2.17 (2H, t, J=8 Hz), 2.50-3.10 (9H, m), 4.60-4.70 (1H, m), 7.00-7.60 (11H, m)

MS (m/z): 450 (M+1)

### EXAMPLE 65

The following compounds were obtained according to a similar manner to that of Example 33 following a similar manner to that of Example 37.

### (1) 5-[[(7S)-7-[[(2R)-2-(6-Chloro-3-pyridyl)-2-hydroxyethyl]amino]-5,6,7,8-tetrahydro-2-naphthalenyl]-oxy]-2-(methylamino)benzoic acid dihydrochloride

NMR (200 MHz, DMSO-d₆, δ): 1.8-3.8 (9H, m), 2.84 (3H, s), 5.05 (1H, m), 6.5-6.9 (4H, m), 7.0-7.2 (2H, m), 7.38 (1H, d, J=2.8 Hz), 7.57 (1H, d, J=8.4 Hz), 7.90 (1H, d, J=2.4 Hz)

MS (m/z): 468 (M+1)

### (2) 5-[[(7S)-7-[[(2R)-2-(6-Chloro-3-pyridyl)-2-hydroxyethyl]amino]-5,6,7,8-tetrahydro-2-naphthalenyl]-oxy]-2-methylbenzoic Acid Hydrochloride

NMR (200 MHz, DMSO-d₆, δ): 1.8-3.8 (9H, m), 2.44 (3H, s), 5.15 (1H, m), 6.6-7.4 (6H, m), 7.56 (1H, d, J=8.4 Hz), 7.93 (1H, dd, J=2.2, 8.4 Hz), 8.47 (1H, m), 9.02 (1H, m), 9.38 (1H, m)

MS (m/z): 453 (M+1)

### (3) 2-(Acetylamino)-5-[[(7S)-7-[[(2R)-2-(6-chloro-3-pyridyl)-2-hydroxyethyl]amino]-5,6,7,8-tetrahydro-2-naphthalenyl]oxy]benzoic Acid Hydrochloride

NMR (200 MHz, DMSO-d₆, δ): 1.8-2.0 (1H, m), 2.11 (3H, s), 2.1-3.0 (3H, m), 3.0-4.0 (5H, m), 5.15 (1H, m), 6.6-7.4 (6H, m), 7.90 (1H, m), 8.1-8.5 (2H, m), 9.02 (1H, m), 9.44 (1H, m)

MS (m/z): 494 (M−1)

### (4) 5-[[(7S)-7-[[(2R)-2-Hydroxy-2-(3-pyridyl)ethyl]amino]-5,6,7,8-tetrahydro-2-naphthalenyl]oxy]-2-methoxybenzoic Acid Dihydrochloride

NMR (200 MHz, DMSO-d₆, δ): 1.8-2.0 (1H, m), 3.0-4.0 (5H, m), 3.80 (3H, s), 5.14 (1H, m), 6.5-7.3 (5H, m), 7.7-7.9 (1H, m), 8.2-8.4 (1H, m), 8.7-8.9 (3H, m)

MS (m/z): 435 (M+1)

### (5) 3-[[(7S)-7-[[(2R)-2-Hydroxy-2-(3-pyridyl)ethyl]amino]-5,6,7,8-tetrahydro-2-naphthalenyl]oxy]-5-(methylamino)-benzoic Acid Trihydrochloride

NMR (200 MHz, DMSO-d₆, δ): 1.8-2.0 (1H, m), 2.69 (3H, m), 3.0-4.0 (5H, m), 5.37 (1H, m), 6.5-7.2 (6H, m), 8.0-8.2 (1H, m), 8.6-8.7 (1H, m), 8.8-9.0 (2H, m), 9.30 (1H, m), 9.57 (1H, m)

MS (m/z): 434 (M+1)

### (6) 3-[[(7S)-7-[[(2R)-2-(3-Chlorophenyl)-2-hydroxyethyl]-amino]-5,6,7,8-tetrahydro-2-naphthalenyl]oxy]-5-(3,4,5,6-tetrahydro-2H-pyran-4-yloxy)benzoic Acid Hydrochloride

NMR (200 MHz, DMSO-d₆, δ): 1.5-2.2 (5H, m), 2.1-3.0 (3H, m), 3.0-3.8 (8H, m), 4.97 (1H, m), 6.33 (1H, m), 6.8-7.0 (4H, m), 7.18 (2H, d, J=8.4 Hz), 7.3-7.6 (4H, m)

MS (m/z): 538 (M+1)

### (7) 3-[[(7S)-7-[[(2R)-2-(3-Chlorophenyl)-2-hydroxyethyl]-amino]-5,6,7,8-tetrahydro-2-naphthalenyl]oxy]-5-(methylamino)benzoic acid dihydrochloride

NMR (200 MHz, DMSO-d₆, δ): 1.8-2.0 (1H, m), 2.1-3.0 (3H, m), 2.69 (3H, m), 3.0-3.8 (6H, m), 5.09 (1H, m), 6.5-7.5 (10H, m), 8.27 (1H, m), 8.95 (1H, m), 9.50 (1H, m)

MS (m/z): 467 (M+1)

### (8) 3-[[(7S)-7-[[(2R)-2-(3-Chlorophenyl)-2-hydroxyethyl]-amino]-5,6,7,8-tetrahydro-2-naphthalenyl]oxy]-5-(3,4,5,6-tetrahydro-2H-pyran-4-ylamino)benzoic Acid Dihydrochloride

NMR (200 MHz, DMSO-d₆, δ): 1.5-2.2 (5H, m), 2.1-3.0 (3H, m), 3.0-3.8 (8H, m), 4.66 (1H, m), 4.97 (1H, m), 6.33 (1H, m), 6.8-7.0 (4H, m), 7.18 (2H, d, J=8.4 Hz), 7.3-7.6 (4H, m)

MS (m/z): 537 (M+1)

### (9) 3-[[(7S)-7-[[(2R)-2-(4-Chlorophenyl)-2-hydroxyethyl]-amino]-5,6,7,8-tetrahydro-2-naphthalenyl]oxy]-5-(3,4,5,6-tetrahydro-2H-pyran-4-ylamino)benzoic Acid Dihydrochloride

NMR (200 MHz, DMSO-d₆, δ): 1.5-2.2 (5H, m), 2.1-3.0 (3H, m), 3.0-3.8 (8H, m), 4.66 (1H, m), 5.05 (1H, m), 6.8-7.2 (6H, m), 7.2-7.6 (4H, m), 8.90 (1H, m), 9.25 (1H, m)

MS (m/z): 537 (M+1)

### (10) 2-Amino-5-[[(7S)-7-[[(2R)-2-(4-chlorophenyl)-2-hydroxyethyl]amino]-5,6,7,8-tetrahydro-2-naphthalenyl]oxy]benzoic Acid Dihydrochloride

NMR (200 MHz, DMSO-d₆, δ): 1.8-2.4 (4H, m), 2.7-3.8 (5H, m), 5.07 (1H, m), 6.5-7.5 (9H, m), 8.97 (1H, m), 9.51 (1H, m)

MS (m/z): 451 (M−1)

### (11) 5-[[(7S)-7-[[(2R)-2-(4-Chlorophenyl)-2-hydroxyethyl]-amino]-5,6,7,8-tetrahydro-2-naphthalenyl]oxy]-2-methylbenzoic Acid Hydrochloride

NMR (200 MHz, DMSO-d₆, δ): 1.8-2.3 (3H, m), 2.5-3.8 (6H, m), 2.49 (3H, s), 5.05 (1H, m), 6.2-7.5 (10H, m)

MS (m/z): 450 (M−1)

### (12) 2-(Acetylamino)-5-[[(7S)-7-[[(2R)-2-(6-chloro-3-pyridyl)-2-hydroxyethyl]amino]-5,6,7,8-tetrahydro-2-naphthalenyl]oxy]benzoic Acid Hydrochloride

NMR (200 MHz, DMSO-d₆, δ): 1.8-2.3 (3H, m), 2.5-3.8 (6H, m), 2.11 (3H, s), 5.02 (1H, m), 6.2-7.5 (10H, m)

MS (m/z): 493 (M−1)

### (13) 6-[4-[(7S)-7-[[(2R)-2-(3-Chlorophenyl)-2-hydroxyethyl]-amino]-5,6,7,8-tetrahydro-2-naphthalenyl]phenoxy]-nicotinic Acid Hydrochloride

NMR (200 MHz, DMSO-d₆, δ): 1.8-2.2 (4H, m), 2.6-3.5 (5H, m), 5.10 (1H, m), 7.0-7.7 (12H, m), 8.28 (1H, dd, J=2.4, 8.6 Hz), 8.67 (1H, d, J=2.4 Hz), 9.04 (1H, s), 9.52 (1H, s)

MS (m/z): 513 (M−1)

### (14) 2-[4-[(7S)-7-[[(2R)-2-(3-Chlorophenyl)-2-hydroxyethyl]-amino]-5,6,7,8-tetrahydro-2-naphthalenyl]phenoxy]-nicotinic Acid Dihydrochloride

NMR (200 MHz, DMSO-d₆, δ): 1.8-2.2 (4H, m), 2.6-3.5 (5H, m), 5.07 (1H, m), 7.0-7.7 (12H, m), 8.2 (2H, m), 9.00 (1H, s), 9.33 (1H, s)

MS (m/z): 513 (M−1)

### (15) 4-[4-[[(7S)-7-[[(2R)-2-(3-Chlorophenyl)-2-hydroxyethyl]amino]-5,6,7,8-tetrahydro-2-naphthalenyl]-oxy]phenoxy]benzoic Acid Hydrochloride

NMR (200 MHz, DMSO-d₆, δ): 1.8-2.2 (4H, m), 2.6-3.5 (5H, m), 5.03 (1H, m), 6.35 (1H, m), 6.8-7.5 (12H, m), 7.94 (2H, d, J=8.4 Hz)

MS (m/z): 528 (M−1)

### (16) 2-[4-[[(7S)-7-[[(2R)-2-(3-Chlorophenyl)-2-hydroxyethyl]amino]-5,6,7,8-tetrahydro-2-naphthalenyl]-oxy]phenoxy]benzoic Acid Hydrochloride

NMR (200 MHz, DMSO-d₆, δ): 1.8-2.2 (4H, m), 2.6-3.5 (5H, m), 5.04 (1H, m), 6.7-7.8 (15H, m)

MS (m/z): 530 (M+1)

### (17) 4-[3-[[(7S)-7-([(2R)-2-(3-Chlorophenyl)-2-hydroxyethyl]amino]-5,6,7,8-tetrahydro-2-naphthalenyl]-oxy]phenyl]benzoic Acid Hydrochloride

NMR (200 MHz, DMSO-d₆, δ): 1.8-2.2 (4H, m), 2.6-3.5 (5H, m), 5.04 (1H, m), 6.7-7.5 (11H, m), 7.77 (2H, d, J=8.4 Hz), 8.00 (2H, d, J=8.4 Hz)

MS (m/z): 512 (M−1)

### (18) 4-[3-[[(7S)-7-[[(2R)-2-(3-Chlorophenyl)-2-hydroxyethyl]amino]-5,6,7,8-tetrahydro-2-naphthalenyl]-oxy]phenoxy]benzoic Acid Hydrochloride

NMR (200 MHz, DMSO-d₆, δ): 1.8-2.2 (4H, m), 2.6-3.5 (5H, m), 5.05 (1H, m), 6.7-7.7 (13H, m), 7.94 (2H, d, J=8.4 Hz)

MS (m/z): 528 (M−1)

### (19) 2-[3-[[(7S)-7-[[(2R)-2-(3-Chlorophenyl)-2-hydroxyethyl]amino)-5,6,7,8-tetrahydro-2-naphthalenyl]-oxy]phenoxy]benzoic Acid Hydrochloride

NMR (200 MHz, DMSO-d₆, δ): 1.8-2.2 (4H, m), 2.6-3.5 (5H, m), 5.05 (1H, m), 6.5-7.8 (13H, m), 7.81 (2H, d, J=8.4 Hz)

MS (m/z): 530 (M+1)

### (20) 3-[3-[[(7S)-7-[[(2R)-2-(3-Chlorophenyl)-2-hydroxyethyl]amino)-5,6,7,8-tetrahydro-2-naphthalenyl]-oxy]phenyl]benzoic Acid Hydrochloride

NMR (200 MHz, DMSO-d₆, δ): 1.8-2.2 (4H, m), 2.6-3.5 (5H, m), 5.04 (1H, m), 6.7-7.8 (12H, m), 7.92 (2H, m), 8.12 (1H, s)

MS (m/z): 514 (M+1)

### (21) 3-[[(7S)-7-[[(2R)-2-(3-Chlorophenyl)-2-hydroxyethyl]-amino]-5,6,7,8-tetrahydro-2-naphthalenyl]oxy]-5-phenoxybenzoic Acid Hydrochloride

MS (m/z): 530 (M+1)

### (22) 3-[[(7S)-7-[[(2R)-2-(3-Chlorophenyl)-2-hydroxyethyl]-amino]-5,6,7,8-tetrahydro-2-naphthalenyl]oxy]-5-anilinobenzoic Acid Dihydrochloride

MS (m/z): 529 (M+1)

### (23) 3-[[(7S)-7-[[(2R)-2-(3-Chlorophenyl)-2-hydroxyethyl]-amino]-5,6,7,8-tetrahydro-2-naphthalenyl]oxy]-5-propoxybenzoic Acid Hydrochloride

MS (m/z): 496 (M+1)

### (24) 3-[[(7S)-7-[[(2R)-2-(3-Chlorophenyl)-2-hydroxyethyl]-amino]-5,6,7,8-tetrahydro-2-naphthalenyl]oxy]-5-(propylamino)benzoic Acid Dihydrochloride

MS (m/z): 495 (M+1)

### (25) 3-[[(7S)-7-[[(2R)-2-(3-Chlorophenyl)-2-hydroxyethyl]-amino]-5,6,7,8-tetrahydro-2-naphthalenyl]oxy]-6-propoxybenzoic Acid Hydrochloride

MS (m/z): 496 (M+1)

### EXAMPLE 66

The following compounds were obtained according to a similar manner to that of Example 33 following a similar manner to that of Example 37.

### (1) 3-Amino-5-[[(7S)-7-[[(2R)-2-(6-chloro-3-pyridyl)-2-hydroxyethyl]amino]-5,6,7,8-tetrahydro-2-naphthalenyl]-oxy]benzoic Acid Dihydrochloride

NMR (200 MHz, DMSO-d₆, δ): 1.8-2.0 (1H, m), 2.11 (3H, s), 3.0-4.0 (5H, m), 5.15 (1H, m), 6.5-7.0 (4H, m), 7.0-7.2 (1H, m), 7.43 (1H, s), 7.57 (2H, d, J=8.4 Hz), 7.93 (1H, d, J=8.4 Hz), 8.48 (1H, m), 9.01 (1H, m), 9.36 (1H, m)

MS (m/z): 451 (M−1)

### (2) 3-[[(7S)-7-[[(2R)-2-(6-Chloro-3-pyridyl)-2-hydroxyethyl]amino]-5,6,7,8-tetrahydro-2-naphthalenyl]-oxy]-5-(dimethylamino)benzoic Acid Dihydrochloride

NMR (200 MHz, DMSO-d₆, δ): 1.8-2.0 (1H, m), 2.96 (6H, s), 3.0-4.0 (5H, m), 5.15 (1H, m), 6.5-7.3 (6H, m), 7.56 (1H, d, J=8.4 Hz), 7.91 (1H, m), 8.46 (1H, m), 9.01 (1H, m), 9.58 (1H, m)

MS (m/z): 482 (M+1)

### (3) 3-[[(7S)-7-[[(2R)-2-(6-Chloro-3-pyridyl)-2-hydroxyethyl]amino]-5,6,7,8-tetrahydro-2-naphthalenyl]-oxy]-5-(3,4,5,6-tetrahydro-2H-pyran-4-ylamino)benzoic Acid Dihydrochloride

NMR (200 MHz, DMSO-d₆, δ): 1.8-2.0 (1H, m), 3.0-4.0 (5H, m), 5.05 (1H, m), 6.5-7.3 (6H, m), 7.57 (1H, d, J=8.4 Hz), 7.91 (1H, m), 8.46 (1H, m), 9.01 (1H, m), 9.58 (1H, m)

MS (m/z): 536 (M−1)

### (4) 3-[[(7S)-7-[[(2R)-2-(6-Chloro-3-pyridyl)-2-hydroxyethyl)amino]-5,6,7,8-tetrahydro-2-naphthalenyl]-oxy]-5-[(methoxycarbonyl)amino]benzoic Acid Hydrochloride

NMR (200 MHz, DMSO-d₆, δ): 1.8-2.0 (1H, m), 3.0-4.0 (5H, m), 3.40 (3H, s), 5.15 (1H, m), 6.4-7.3 (4H, m), 7.4-8.0 (4H, m), 8.48 (1H, m), 9.02 (1H, m), 9.41 (1H, m)

MS (m/z): 510 (M−1)

### (5) 3-[[(7S)-7-[[(2R)-2-(6-Chloro-3-pyridyl)-2-hydroxyethyl]amino]-5,6,7,8-tetrahydro-2-naphthalenyl]-oxy]-5-(2-furoylamino)benzoic Acid Hydrochloride

NMR (200 MHz, DMSO-d₆, δ): 1.8-2.0 (1H, m), 3.0-4.0 (5H, m), 5.12 (1H, m), 6.79 (1H, m), 6.7-7.0 (2H, m), 7.1-7.2 (2H, m), 7.39 (1H, d, J=3.4 Hz), 7.57 (1H, d, J=8.4 Hz), 7.80 (1H, m), 7.8-8.0 (2H, m), 8.11 (1H, m), 8.47 (1H, m), 9.02 (1H, m), 9.30 (1H, m)

MS (m/z): 546 (M−1)

### (6) 3-(Benzoylamino)-5-[[(7S)-7-[[(2R)-2-(6-chloro-3-pyridyl)-2-hydroxyethyl]amino]-5,6,7,8-tetrahydro-2-naphthalenyl]oxy]benzoic Acid Hydrochloride

NMR (200 MHz, DMSO-d₆, δ): 1.8-2.0 (1H, m), 3.0-4.0 (5H, m), 5.14 (1H, m), 6.5-7.2 (4H, m), 7.4-8.0 (8H, m), 8.20 (1H, m), 8.43 (1H, m), 9.01 (1H, m), 9.39 (1H, m)

MS (m/z): 559 (M+1)

### (7) 3-[[(Benzyloxy)carbonyl]amino]-5-[[(7S)-7-[[(2R)-2-(6-chloro-3-pyridyl)-2-hydroxyethyl]amino]-5,6,7,8-tetrahydro-2-naphthalenyl]oxy]benzoic Acid Hydrochloride

NMR (200 MHz, DMSO-d₆, δ): 1.8-2.0 (1H, m), 3.0-4.0 (5H, m), 5.14 (1H, m), 5.15 (2H, s), 6.3-7.2 (5H, m), 7.2-7.7 (5H, m), 7.8-8.0 (2H, m), 8.48 (1H, m), 8.97 (1H, in), 9.27 (1H, m)

MS (m/z): 588 (M+1)

### (8) 3-(Dimethylamino)-5-[[(7S)-7-[[(2R)-2-hydroxy-2-(3-pyridyl)ethyl]amino]-5,6,7,8-tetrahydro-2-naphthalenyl]oxy]benzoic Acid Trihydrochloride

NMR (200 MHz, DMSO-d₆, δ): 1.8-2.0 (1H, m), 2.94 (6H, m), 3.0-4.0 (5H, m), 5.37 (1H, m), 6.5-7.2 (6H, m), 7.9-8.1 (1H, m), 8.6-8.7 (1H, m), 8.8-9.1 (3H, m), 9.20 (1H, m), 9.50 (1H, m)

MS (m/z): 446 (M−1)

### (9) 3-[[(7S)-7-[[(2R)-2-Hydroxy-2-(3-pyridyl)ethyl]amino]-5,6,7,8-tetrahydro-2-naphthalenyl]oxy]-5-(3,4,5,6-tetrahydro-2H-pyran-4-ylamino)benzoic Acid Trihydrochloride

NMR (200 MHz, DMSO-d₆, δ): 1.8-2.0 (1H, m), 3.0-4.0 (5H m), 5.37 (1H, m), 6.8-7.4 (6H, m), 7.9-8.1 (1H, m), 8.49 (1H, d, J=8.4 Hz), 8.8-9.1 (3H, m), 9.19 (1H, m), 9.41 (1H, m)

MS (m/z): 502 (M−1)

### (10) 3-[[(7S)-7-[[(2R)-2-Hydroxy-2-(3-pyridyl)ethyl]amino]-5,6,7,8-tetrahydro-2-naphthalenyl)oxy]-5-[(methoxycarbonyl)amino)benzoic Acid Dihydrochloride

NMR (200 MHz, DMSO-d₆, δ): 1.8-2.0 (1H, m), 3.0-4.0 (5H m), 3.60 (3H, s), 5.21 (1H, m), 6.8-7.4 (4H, m), 7.4-7.8 (2H, m), 8.1-8.3 (1H, m), 8.6-8.9 (2H, m)

MS (m/z): 478 (M+1)

### (11) 3-(2-Furoylamino)-5-[[(7S)-7-[[(2R)-2-hydroxy-2-(3-pyridyl)ethyl]amino]-5,6,7,8-tetrahydro-2-naphthalenyl]oxy]benzoic Acid Dihydrochloride

NMR (200 MHz, DMSO-d₆, δ): 1.8-2.0 (1H, m), 3.0-4.0 (5H m), 5.25 (1H, m), 6.7-7.2 (4H, m), 7.71 (1H, m), 7.8-8.0 (1H, m), 8.09 (1H, s), 8.35 (1H, d, J=8.4 Hz), 8.7-9.0 (2H, m), 9.1 (1H, m), 9.46 (1H, m), 10.01 (1H, s)

MS (m/z): 524 (M+1)

### (12) 3-(Benzoylamino)-5-[[(7S)-7-[[(2R)-2-hydroxy-2-(3-pyridyl)ethyl]amino]-5,6,7,8-tetrahydro-2-naphthalenyl]oxy]benzoic Acid Dihydrochloride

NMR (200 MHz, DMSO-d₆, δ): 1.8-2.0 (1H, m), 3.0-4.0 (5H m), 5.29 (1H, m), 6.7-7.2 (4H, m), 7.5-7.7 (3H, m), 7.8-8.0 (4H, m), 8.18 (1H, s), 8.43 (1H, d, J=8.4 Hz), 9.15 (1H, m), 9.36 (1H, m), 10.51 (1H, s)

MS (m/z): 522 (M−1)

### (13) 3-Amino-5-[[(7S)-7-[[(2R)-2-hydroxy-2-(3-pyridyl)ethyl)amino]-5,6,7,8-tetrahydro-2-naphthalenyl]oxy]-benzoic Acid Trihydrochloride

NMR (200 MHz, DMSO-d₆, δ): 1.8-2.0 (1H, m), 3.0-4.0 (5H m), 5.32 (1H, m), 6.5-7.2 (6H, m), 8.0-8.2 (1H, m), 8.6-8.7 (1H, d, J=8.4 Hz), 8.85 (1H, d, J=8.4 Hz), 8.93 (1H, m), 9.20 (1H, m), 9.45 (1H, m)

MS (m/z): 420 (M+1)

### (14) 3-Amino-5-[[(7S)-7-[[(2R)-2-(4-chlorophenyl)-2-hydroxyethyl]amino]-5,6,7,8-tetrahydro-2-naphthalenyl]oxy]benzoic Acid Dihydrochloride

NMR (200 MHz, DMSO-d₆, δ): 1.8-2.3 (3H, m), 2.5-3.8 (6H, m), 5.02 (1H, m), 6.2-7.4 (10H, m), 8.87 (1H, m), 9.19 (1H, m)

MS (m/z): 452 (M−1)

### (15) 3-[[(7S)-7-[[(2R)-2-(4-Chlorophenyl)-2-hydroxyethyl]-amino]-5,6,7,8-tetrahydro-2-naphthalenyl]oxy]-5-(2-furoylamino)benzoic Acid Hydrochloride

NMR (200 MHz, DMSO-d₆, δ): 1.8-2.0 (2H, m), 2.1-2.3 (2H, m), 2.5-3.6 (5H, m), 5.05 (1H, m), 6.30 (1H, m), 6.69 (1H, m), 6.8-7.2 (4H, m), 7.3-7.6 (4H, m), 7.80 (1H, s), 7.94 (1H, s), 8.16 (1H, s), 8.92 (1H, m), 9.33 (1H, m)

MS (m/z): 547 (M−1)

### (16) 3-[[(7S)-7-[[(2R)-2-(4-Chlorophenyl)-2-hydroxyethyl]-amino]-5,6,7,8-tetrahydro-2-naphthalenyl]oxy]-5-[(methoxycarbonyl)amino]benzoic Acid Hydrochloride

NMR (200 MHz, DMSO-d₆, δ): 1.8-2.2 (41H, m), 2.8-3.6 (5H, m), 3.66 (3H, s), 5.02 (1H, m), 6.4-7.7 (9H, m), 7.79 (1H, s), 8.87 (1H, m), 9.22 (1H, m)

MS (m/z): 511 (M+1)

### (17) 3-(Benzoylamino)-5-[[(7S)-7-[[(2R)-2-(4-chlorophenyl)-2-hydroxyethyl]amino]-5,6,7,8-tetrahydro-2-naphthalenyl]oxy]benzoic Acid Hydrochloride

NMR (200 MHz, DMSO-d₆, δ): 1.8-2.2 (4H, m), 2.6-3.5 (5H, m), 5.05 (1H, m), 6.3-7.7 (11H, m), 7.83 (1H, s), 7.94 (1H, d, J=8.4 Hz), 9.19 (1H, m)

MS (m/z): 557 (M+1)

### EXAMPLE 67

The following compounds were obtained according to a similar manner to that of Preparation 4 following a similar manner to that of Example 37.

### (1) 4-[(7S)-7-[[(2R)-2-(4-Chlorophenyl)-2-hydroxyethyl]-amino]-5,6,7,8-tetrahydro-2-naphthalenyl]-2-(cyclohexyloxy)benzoic Acid Hydrochloride

NMR (200 MHz, DMSO-d₆, δ): 1.2-3.3 (19H, m), 4.63 (1H, m), 5.04 (1H, m), 6.5-7.2 (3H, m), 7.2-7.8 (8H, m), 8.95 (1H, m), 9.19 (1H, m)

MS (m/z): 521 (M+1)

### (2) 4-[(7S)-7-[[(2R)-2-(4-Chlorophenyl)-2-hydroxyethyl]-amino]-5,6,7,8-tetrahydro-2-naphthalenyl]-2-(cyclohexyloxy)benzoic Acid Hydrochloride

NMR (200 MHz, DMSO-d₆, δ): 1.5-2.4 (13H, m), 2.7-3.5 (6H, m), 4.65 (1H, m), 5.05 (1H, m), 7.0-7.7 (10H, m), 8.25 (1H, m), 8.95 (1H, m), 9.20 (1H, m)

MS (m/z): 520 (M+1)

### (3) 4-[(7S)-7-[[(2R)-2-(4-Chlorophenyl)-2-hydroxyethyl]-amino]-5,6,7,8-tetrahydro-2-naphthalenyl]-2-isopropoxybenzoic Acid Hydrochloride

NMR (200 MHz, DMSO-d₆, δ): 1.25 (6H, d, J=6.0 Hz), 1.5-3.5 (10H, m), 4.77 (1H, m), 5.02 (1H, m), 6.2-7.0 (3H, m), 7.1-7.6 (5H, m), 7.68 (2H, d, J=8.4 Hz)

MS (m/z): 480 (M+1)

### (4) 2-(Cyclohexyloxy)-4-[(7S)-7-[[(2R)-2-hydroxy-2-phenylethyl]amino]-5,6,7,8-tetrahydro-2-naphthalenyl]-benzoic Acid Hydrochloride

NMR (200 MHz, DMSO-d₆, δ): 1.5-2.4 (13H, m), 2.7-3.5 (6H, m), 4.63 (1H, m), 5.04 (1H, m), 7.0-7.6 (9H, m), 7.69 (2H, d, J=8.4 Hz), 8.25 (1H, m)

MS (m/z): 486 (M+1)

### (5) 4-[(7S)-7-[[(2R)-2-Hydroxy-2-phenylethyl]amino]-5,6,7,8-tetrahydro-2-naphthalenyl]-2-isopropoxybenzoic Acid Hydrochloride

NMR (200 MHz, DMSO-d₆, δ): 1.26 (6H, d, J=6.0 Hz), 1.5-3.5 (10H, m), 4.80 (1H, m), 5.07 (1H, m), 6.26 (1H, m), 7.1-7.6 (8H, m), 7.68 (2H, d, J=8.4 Hz)

MS (m/z): 446 (M+1)

### (6) 4-[4-[(7S)-7-[[(2R)-2-(3-Chlorophenyl)-2-hydroxethyl]-amino]-5,6,7,8-tetrahydro-2-naphthalenyl]phenoxy]-benzoic Acid Hydrochloride

NMR (200 MHz, DMSO-d₆, δ): 1.8-2.2 (4H, m), 2.6-3.5 (5H, m), 5.05 (1H, m), 6.36 (1H, m), 7.0-7.6 (11H, m), 7.69 (2H, d, J=8.4 Hz), 7.96 (2H, d, J=8.4 Hz)

MS (m/z): 512 (M−1)

### (7) 3-[4-[(7S)-7-[[(2R)-2-(3-Chlorophenyl)-2-hydroxethyl]-amino]-5,6,7,8-tetrahydro-2-naphthalenyl]phenoxy]-benzoic Acid Hydrochloride

NMR (200 MHz, DMSO-d₆, δ): 1.8-2.2 (4H, m), 2.6-3.5 (5H, m), 5.05 (1H, m), 6.36 (1H, m), 7.0-7.8 (15H, m)

MS (m/z): 512 (M−1)

### (8) 2-[4-[(7S)-7-[[(2R)-2-(3-Chlorophenyl)-2-hydroxethyl]-amino]-5,6,7,8-tetrahydro-2-naphthalenyl]phenoxy]-benzoic Acid Hydrochloride

NMR (200 MHz, DMSO-d₆, δ): 1.8-2.2 (4H, m), 2.6-3.5 (5H, m), 5.10 (1H, m), 6.36 (1H, m), 6.8-8.0 (15H, m)

MS (m/z): 512 (M−1)

### (9) 3-[3-[(7S)-7-[[(2R)-2-(3-Chlorophenyl)-2-hydroxethyl]-amino]-5,6,7,8-tetrahydro-2-naphthalenyl]phenoxy]-benzoic Acid Hydrochloride

NMR (200 MHz, DMSO-d₆, δ): 1.8-2.2 (4H, m), 2.6-3.5 (5H, m), 5.10 (1H, m), 6.36 (1H, m), 6.8-8.0 (15H, m)

MS (m/z): 512 (M−1)

### (10) 4-[3-[(7S)-7-[[(2R)-2-(3-Chlorophenyl)-2-hydroxethyl]-amino]-5,6,7,8-tetrahydro-2-naphthalenyl]phenoxy]-benzoic Acid Hydrochloride

NMR (200 MHz, DMSO-d₆, δ): 1.8-2.2 (4H, m), 2.6-3.5 (5H, m), 5.10 (1H, m), 6.36 (1H, m), 6.8-8.0 (15H, m)

MS (m/z): 512 (M−1)

### (11) 2-(3-[(7S)-7-[[(2R)-2-(3-Chlorophenyl)-2-hydroxethyl]-amino]-5,6,7,8-tetrahydro-2-naphthalenyl]phenoxy]-benzoic Acid Hydrochloride

NMR (200 MHz, DMSO-d₆, δ): 1.8-2.2 (4H, m), 2.6-3.5 (5H, m), 5.03 (1H, m), 6.37 (1H, m), 6.8-8.0 (15H, m)

MS (m/z): 512 (M−1)

### (12) 4-[(7S)-7-[[(2R)-2-Hydroxy-2-(3-pyridyl)ethyl]amino]-5,6,7,8-tetrahydro-2-naphthalenyl]-2-phenoxybenzoic Acid Dihydrochloride

MS (m/z): 481 (M+1)

### (13) 4-[(7S)-7-[[(2R)-2-(4-Chlorophenyl)-2-hydroxethyl]-amino]-5,6,7,8-tetrahydro-2-naphthalenyl]-2-propoxybenzoic Acid Hydrochloride

MS (m/z): 480 (M+1)

### (14) 4-[(7S)-7-[[(2R)-2-(4-Chlorophenyl)-2-hydroxethyl]-amino]-5,6,7,8-tetrahydro-2-naphthalenyl]-2-phenoxybenzoic Acid Hydrochloride

MS (m/z): 514 (M+1)

### (15) 4-[(7S)-7-[[(2R)-2-Phenyl-2-hydroxyethyl]amino]-5,6,7,8-tetrahydro-2-naphthalenyl]-2-propoxybenzoic Acid Hydrochloride

MS (m/z): 446 (M+1)

### (16) 4-[(7S)-7-[[(2R)-2-Phenyl-2-hydroxyethyl]amino]-5,6,7,8-tetrahydro-2-naphthalenyl]-2-phenoxybenzoic Acid Hydrochloride

MS (m/z): 480 (M+1)

### EXAMPLE 68

The following compounds were obtained according to a similar manner to that of Example 17 following a similar manner to that of Example 19.

### (1) Sodium 4-[(7S)-7-[[(2R)-2-(3,5-dichlorophenyl)-2-hydroxyethyl]amino]-5,6,7,8-tetrahydro-2-naphthalenyl]-benzoate

NMR (200 MHz, DMSO-d₆, δ): 1.8-3.0 (9H, m), 4.66 (1H, m), 7.0-7.2 (1H, m), 7.2-7.7 (7H, m), 7.8-8.0 (2H, m)

MS (m/z): 456 (M+1)

### (2) Sodium 4-[(7S)-7-[[(2R)-2-(3,4-dimethylphenyl)-2-hydroxyethyl]amino]-5,6,7,8-tetrahydro-2-naphthalenyl]-benzoate

NMR (200 MHz, DMSO-d₆, δ): 1.8-3.0 (9H, m), 2.18 (3H, s), 2.20 (3H, s), 4.54 (1H, m), 7.0-7.2 (4H, m), 7.2-7.5 (4H, m), 7.8-8.0 (2H, m)

MS (m/z): 416 (M+1)

### (3) Sodium 4-[(7S)-7-[[(2R)-2-(2-chlorophenyl)-2-hydroxyethyl]amino]-5,6,7,8-tetrahydro-2-naphthalenyl]-benzoate

NMR (200 MHz, DMSO-d₆, δ): 1.8-3.0 (9H, m), 4.97 (1H, m), 7.0-7.7 (9H, m), 7.8-8.0 (2H, m)

MS (m/z): 420 (M+1)

### (4) Sodium 4-[(7S)-7-[[(2R)-2-(4-trifluorophenyl)-2-hydroxyethyl]amino]-5,6,7,8-tetrahydro-2-naphthalenyl]-benzoate

NMR (200 MHz, DMSO-d₆, δ): 1.8-3.2 (9H, m), 4.73 (1H, m), 7.11 (1H, d, J=8.6 Hz), 7.3-7.8 (8H, m), 7.88 (2H, d, J=8.2 Hz)

MS (m/z): 456 (M+1)

### (5) Sodium 4-[(7S)-7-[[(2R)-2-(4-cyanophenyl)-2-hydroxyethyl]amino]-5,6,7,8-tetrahydro-2-naphthalenyl]-benzoate

NMR (200 MHz, DMSO-d₆, δ): 1.4-3.0 (9H, m), 4.72 (1H, m), 7.12 (1H, d, J=8.2 Hz), 7.2-7.6 (6H, m), 8.82 (2H, d, J=8.4 Hz), 7.92 (2H, d, J=8.4 Hz)

MS (m/z): 413 (M+1)

### (6) Sodium 4-[(7S)-7-[[(2R)-2-(3,4-dichlorophenyl)-2-hydroxyethyl]amino)-5,6,7,8-tetrahydro-2-naphthalenyl]-benzoate

NMR (200 MHz, DMSO-d₆, δ): 1.8-3.0 (9H, m), 4.66 (1H, m), 7.0-7.2 (1H, m), 7.2-7.9 (9H, m)

MS (m/z): 472 (M+1)

### 7) Sodium 4-[(7S)-7-[[(2R)-2-(3-fluoro-4-chlorophenyl)-2-hydroxyethyl]amino]-5,6,7,8-tetrahydro-2-naphthalenyl]-benzoate

NMR (200 MHz, DMSO-d₆, δ): 1.5-3.0 (9H, m), 4.68 (1H, m), 7.0-7.5 (8H, m), 7.89 (2H, d, J=8.4 Hz)

MS (m/z): 483 (M−1)

### (8) Sodium 4-[(7S)-7-[[(2R)-2-(3-trifluoro-4-chlorophenyl)-2-hydroxyethyl]amino]-5,6,7,8-tetrahydro-2-naphthalenyl]benzoate

NMR (200 MHz, DMSO-d₆, δ): 1.8-3.0 (9H, m), 4.66 (1H, m), 7.0-7.2 (1H, m), 7.2-8.0 (9H, m)

MS (m/z): 488 (M−1)

### (9) Sodium 4-[(7S)-7-[[(2R)-2-(4-isopropylphenyl)-2-hydroxyethyl]amino]-5,6,7,8-tetrahydro-2-naphthalenyl]-benzoate

NMR (200 MHz, DMSO-d₆, δ): 1.22 (6H, d, J=6.8 Hz), 1.8-3.0 (10H, m), 4.66 (1H, m), 7.0-7.8 (9H, m), 7.8-8.0 (2H, m)

MS (m/z): 430 (M+1)

### EXAMPLE 69

To a solution of 3-[(7S)-7-[N-[(2R)-2-(3-chlorophenyl)-2-hydroxyethyl]-N-(tert-butoxycarbonyl)amino]-5,6,7,8-tetrahydro-2-naphthalenyl]benzoic acid (100 mg) in N,N-dimethylformamide (10 ml) were added methylsulfonamide (50 mg) and 1-[3-(dimethylamino)propyl]-3-ethylcarbodiimide hydrochloride (100 mg), and dimethylaminopyridine (60 mg) at room temperature. After stirred for 24 hours, the mixture was diluted with a mixture of ethyl acetate and water and the organic layer was washed with brine, dried over magnesium sulfate. The resulting mixture was filtrated and the mother layer was evaporated under pressure. The residue was purified by column chromatography on silica gel to give sulfonamido derivative. The obtained sulfonamide derivative (60 mg) was diluted with 6N hydrogen chloride in dioxane (10 ml) and the mixture was allowed to keep at room temperature for 4 hours. The mixture was evaporated under reduced pressure and the obtained solid was washed with ether to give N-[4-[(7S)-7-[[(2R)-2-(3-chlorophenyl)-2-hydroxyethyl]amino]-5,6,7,8-tetrahydro-2-naphthalenyl]benzoyl]methanesulfonamide hydrochloride (33 mg).

NMR (200 MHz, DMSO-d₆, δ): 1.2-3.3 (9H, m), 3.44 (3H, m), 5.04 (1H, m), 6.33 (1H, m), 7.2-7.6 (7H, m), 7.79 (2H, d, J=8.4 Hz), 8.05 (2H, d, J=8.4 Hz)

(+)ESI-MS (m/z): 497 (M−1)

### EXAMPLE 70

The following compounds were obtained according to a similar manner to that of Example 69.

### (1) N-[4-[(7S)-7-[[(2R)-2-(3-Chlorophenyl)-2-hydroxyethyl]-amino]-5,6,7,8-tetrahydro-2-naphthalenyl]benzoyl]-benzenesulfonamide Hydrochloride

NMR (200 MHz, DMSO-d₆, δ): 1.5-3.3 (9H, m), 3.44 (3H, m), 5.05 (1H, m), 6.38 (1H, m), 7.2-8.1 (14H, m), 8.95 (1H, m), 9.20 (1H, m)

MS (m/z): 559 (M−1)

### (2) N-[4-[(7S)-7-[[(2R)-2-(3-Chlorophenyl)-2-hydroxyethyl]-amino]-5,6,7,8-tetrahydro-2-naphthalenyl]benzoyl]-benzylsulfonamide Hydrochloride

NMR (200 MHz, DMSO-d₆, δ): 1.5-3.3 (9H, m), 3.44 (3H, m), 4.87 (2H, s), 5.08 (1H, m), 6.40 (1H, m), 7.2-7.6 (11H, m), 7.78 (2H, d, J=8.4 Hz), 7.98 (2H, d, J=8.4 Hz), 8.96 (1H, m), 9.24 (1H, m)

MS (m/z): 573 (M−1)

### EXAMPLE 71

The following compounds were obtained according to a similar manner to that of Example 39 following a similar manner to that of Example 37.

### (1) 3-Chloro-2-[[(7S)-7-[[(2R)-2-(3-chlorophenyl)-2-hydroxyethyl]amino]-5,6,7,8-tetrahydro-2-naphthalenyl]oxy]isonicotinic Acid Dihydrochloride

NMR (200 MHz, DMSO-d₆, δ): 1.8-2.0 (1H, m), 2.1-3.0 (3H, m), 3.0-3.8 (6H, m), 5.05 (1H, m), 6.3-7.0 (4H, m), 7.3-7.6 (4H, m), 8.9 (1H, m), 9.2 (1H, s), 9.27 (1H, m)

MS (m/z): 471 (M−1)

### (2) 5-[[(7S)-7-[[(2R)-2-(3-Chlorophenyl)-2-hydroxyethyl]-amino]-5,6,7,8-tetrahydro-2-naphthalenyl]oxy]-2-pyrazinecarboxylic Acid Hydrochloride

NMR (200 MHz, DMSO-d₆, δ): 1.8-2.0 (1H, m), 2.1-3.0 (3H, m), 3.0-3.8 (6H, m), 5.09 (1H, m), 6.9-7.1 (2H, m), 7.20 (1H, d, J=8.4 Hz), 7.3-7.5 (4H, m), 8.61 (1H, s), 8.73 (1H, s)

MS (m/z): 438 (M−1)

### (3) 3-Chloro-2-[[(7S)-7-[[(2R)-2-(4-chlorophenyl)-2-hydroxyethyl]amino]-5,6,7,8-tetrahydro-2-naphthalenyl]oxy]isonicotinic Acid Hydrochloride

NMR (200 MHz, DMSO-d₆, δ): 1.8-2.0 (1H, m), 2.1-3.0 (3H, m), 3.0-3.8 (6H, m), 5.10 (1H, m), 6.8-7.4 (8H, m), 8.29 (1H, d, J=8.4 Hz), 9.06 (1H, m), 9.59 (1H, m)

MS (m/z): 471 (M−1)

### (4) 5-Chloro-6-[(7S)-7-[(2R)-2-(3-chlorophenyl)-2-hydroxyethyl]amino]-5,6,7,8-tetrahydro-2-naphthalenyl]oxy]nicotinic Acid Hydrochloride

NMR (200 MHz, DMSO-d₆, δ): 1.8-2.0 (1H, m), 2.1-3.0 (3H, m), 3.0-3.8 (6H, m), 5.09 (1H, m), 6.38 (12H, m), 6.8-7.5 (7H, m), 8.35 (1H, s), 8.54 (1H, s), 9.02 (1H, m), 9.57 (1H, m)

MS (m/z): 471 (M−1)

### (5) 6-[[(7S)-7-[[(2R)-2-(4-Chlorophenyl)-2-hydroxyethyl]-amino]-5,6,7,8-tetrahydro-2-naphthalenyl]oxy]nicotinic Acid Hydrochloride

NMR (200 MHz, DMSO-d₆, δ): 1.8-2.3 (3H, m), 2.5-3.8 (6H, m), 5.05 (1H, m), 6.2-7.5 (8H, m), 8.27 (1H, m), 8.63 (1H, m), 8.95 (1H, m), 9.34 (1H, m)

MS (m/z): 437 (M−1)

### (6) 4-[6-[[(7S)-7-[[(2R)-2-(3-Chlorophenyl)-2-hydroxyethyl]amino]-5,6,7,8-tetrahydro-2-naphthalenyl]-oxy]-3-pyridyl]benzoic acid hydrochloride

NMR (200 MHz, DMSO-d₆, δ): 1.8-2.2 (4H, m), 2.6-3.5 (5H, m), 5.07 (1H, m), 6.8-7.3 (4H, m), 7.3-7.5 (3H, m), 7.81 (2H, d, J=8.4 Hz), 8.02 (2H, d, J=8.4 Hz), 8.1-8.3 (1H, m), 8.51 (1H, dd, J=2.4 Hz)

MS (m/z): 513 (M−1)

### (7) 3-[6-[[(7S)-7-[[(2R)-2-(3-Chlorophenyl)-2-hydroxyethyl]amino]-5,6,7,8-tetrahydro-2-naphthalenyl]-oxy]-3-pyridyl]benzoic Acid Hydrochloride

NMR (200 MHz, DMSO-d₆, δ): 1.8-2.2 (4H, m), 2.6-3.5 (5H, m), 5.10 (1H, m), 6.8-7.6 (9H, m), 7.8-8.0 (2H, m), 8.0-8.2 (2H, m), 8.47 (1H, m)

MS (m/z): 513 (M−1)

### EXAMPLE 72

Under nitrogen at room temperature, to a mixture of bis(dibenzylideneacetone)palladium(0) (103 mg) and bis(2-diphenylphosphinophenyl)ether (107 mg) was added toluene (20 ml). After being stirred at the same temperature for 15 minutes, (7S)-7-[N-(tert-butoxycarbonyl)-N-[(2R)-2-(3-chlorophenyl)-2-hydroxyethyl]amino]-5,6,7,8-tetrahydro-2-naphthalenyl trifluoromethanesulfonate (1 g), potassium tert-butoxide (0.3 g) and 3-mercaptobenzoic acid (0.3 ml) were added, and the mixture was stirred at 80° C. for 3 hours. The resulting mixture was poured into water and the aqueous mixture was extracted with ethyl acetate. The organic layer was washed successively with water and brine, dried over anhydrous magnesium sulfate and evaporated under reduced pressure. The residue was purified by column chromatography on silica gel to give sulfide derivative. The obtained sulfide derivative (70 mg) was diluted with 6N hydrogen chloride in 1,4-dioxane (10 ml) and the mixture was allowed to keep at room temperature for 4 hours. The mixture was evaporated under reduced pressure and the obtained solid was washed with ether to give 3-[[(7S)-7-[[(2R)-2-(3-chlorophenyl)-2-hydroxyethyl]amino]-5,6,7,8-tetrahydro-2-naphthalenyl]thio]benzoic acid hydrochloride (51 mg).

NMR (200 MHz, DMSO-d₆, δ): 1.8-2.2 (4H, m), 2.6-3.5 (5H, m), 5.02 (1H, m), 6.5-7.8 (11H, m)

MS (m/z): 454 (M+1)

### EXAMPLE 73

Under nitrogen at room temperature, to a mixture of bis(dibenzylideneacetone)palladium(0) (103 mg) and bis(2-diphenylphosphinophenyl)ether (107 mg) was added toluene (20 ml). After being stirred at the same temperature for 15 minutes, (7S)-7-[N-(tert-butoxycarbonyl)-N-[(2R)-2-(3-chlorophenyl)-2-hydroxyethyl]amino]-5,6,7,8-tetrahydro-2-naphthalenyl trifluoromethanesulfonate (1 g), potassium tert-butoxide (0.3 g) and 3-mercaptobenzoic acid (0.3 ml) were added, and the mixture was stirred at 80° C. for 3 hours. The resulting mixture was poured into water and the aqueous mixture was extracted with ethyl acetate. The organic layer was washed successively with water and brine, dried over anhydrous magnesium sulfate and evaporated under reduced pressure. The residue was purified by column chromatography on silica gel to give sulfide derivative. Under nitrogen at 5° C., to a solution of the obtained sulfide (300 mg) in dichloromethane (10 ml) was added m-chloroperoxybenzoic acid (150 mg), and the mixture was stirred at room temperature for 3.5 hours. The resulting mixture was poured into aqueous sodium thiosulfate and the aqueous mixture was extracted with ethyl acetate. The organic layer was washed successively with saturated aqueous sodium bicarbonate twice and brine, dried over anhydrous magnesium sulfate, evaporated under reduced pressure and dried in vacuo to give the sulfoxide derivative. The obtained sulfoxide derivative (100 mg) was diluted with 6N hydrogen chloride in 1,4-dioxane (10 ml) and the mixture was allowed to keep at room temperature for 4 hours. The mixture was evaporated under reduced pressure and the obtained solid was washed with ether to give 3-[[(7S)-7-[[(2R)-2-(3-chlorophenyl)-2-hydroxyethyl]amino]-5,6,7,8-tetrahydro-2-naphthalenyl]-sulfonyl]benzoic acid hydrochloride (700 mg).

NMR (200 MHz, DMSO-d₆, δ): 1.8-2.2 (4H, m), 2.6-3.5 (5H, m), 5.02 (1H, m), 6.38 (1H, m), 7.2-7.8 (7H, m), 8.1-8.3 (3H, m)

MS (m/z): 484 (M−1)

### EXAMPLE 74

The following compound was obtained according to a similar manner to that of Example 73.

### 4-[[(7S)-7-[[(2R)-2-(3-Chlorophenyl)-2-hydroxyethyl]-amino]-5,6,7,8-tetrahydro-2-naphthalenyl]sulfonyl]benzoic Acid Hydrochloride

NMR (200 MHz, DMSO-d₆, δ): 1.8-2.2 (4H, m), 2.6-3.5 (5H, m), 5.03 (1H, m), 6.36 (1H, m), 7.3-7.8 (7H, m), 8.0-8.2 (4H, m)

MS (m/z): 484 (M−1)

### EXAMPLE 75

The following compound was obtained according to a similar manner to that of Preparation 4 following a similar manner to that of Example 37.

### 3-[[(7S)-7-[[(2R)-2-(3-Chlorophenyl)-2-hydroxyethyl]-amino]-5,6,7,8-tetrahydro-2-naphthalenyl]amino]benzoic Acid Dihydrochloride

NMR (200 MHz, DMSO-d₆, δ): 1.8-2.2 (4H, m), 2.6-3.5 (5H, m), 5.02 (2H, m), 6.5-7.8 (11H, m)

MS (m/z): 435 (M−1)

### EXAMPLE 76

To a solution of (7S)-7-[N-(tert-butoxycarbonyl)-N-[(2R)-2-(3-chlorophenyl)-2-hydroxyethyl]amino]-5,6,7,8-tetrahydro-2-naphthalenyl trifluoromethanesulfonate (500 mg) in N,N-dimethylformamide (10 ml) was added methoxycarbonylphenyl acetylene (100 mg), dichlorobis(triphenylphosphine)palladium(II) (50 mg), and triethylamine (100 ml) and the mixture was stirred at 100° C. for 18 hours under nitrogen. The mixture was diluted with ethyl acetate and water. The organic layer was separated, washed with brine, dried over magnesium sulfate and evaporated. The residue was purified by column chromatography on silica gel (hexane/ethyl acetate=5/1) to give acetylene derivative. To a solution of the obtained acetylene derivative in methanol (10 ml) was added 1N sodium hydroxide (5 ml) at room temperature, and the mixture was stirred at the same temperature for 12 hours. The resulting mixture was evaporated under reduced pressure. The residue was diluted with a mixture of ethyl acetate (30 ml) and 1N hydrochloric acid (10 ml), and the organic layer was washed with brine, dried over magnesium sulfate, and evaporated under reduced pressure. The obtained crude was diluted with 6N hydrogen chloride in dioxane (10 ml) and the mixture was allowed to keep at room temperature for 4 hours. The mixture was evaporated under reduced pressure and the obtained solid was washed with ether to give 4-[[(7S)-7-[[(2R)-2-(3-chlorophenyl)-2-hydroxyethyl]amino]-5,6,7,8-tetrahydro-2-naphthalenyl]ethynyl]benzoic acid hydrochloride (150 mg).

NMR (200 MHz, DMSO-d₆, δ): 1.8-2.2 (4H, m), 2.6-3.5 (5H, m), 5.04 (1H, m), 6.38 (1H, m), 7.1-7.5 (7H, m), 7.64 (2H, d, J=8.4 Hz), 7.96 (2H, d, J=8.4 Hz), 8.93 (1H, m), 9.20 (1H, m)

MS (m/z): 446 (M−1)

### EXAMPLE 77

To a mixture of (7S)-7-[N-[(2R)-2-(3-chlorophenyl)-2-hydroxyethyl]-N-(tert-butoxycarbonyl)amino]-2-hydroxy-5,6,7,8-tetrahydronaphthalene (200 mg) in N,N-dimethylformamide (10 ml) were added methyl 4-(bromomethyl)benzoate (100 mg) and potassium carbonate (100 mg) at room temperature, and the mixture was stirred at the same temperature for 12 hours. The residue was diluted with a mixture of ethyl acetate and water, and the organic layer was washed with brine, dried over magnesium sulfate, and evaporated under reduced pressure. The residue was purified by column chromatography on silica gel to give ester derivative. To a solution of the obtained ester derivative in methanol (10 ml) was added 1N sodium hydroxide (5 ml) at room temperature, and the mixture was stirred at the same temperature for 12 hours. The resulting mixture was evaporated under reduced pressure. The residue was diluted with a mixture of ethyl acetate (30 ml) and 1N hydrochloric acid (10 ml), and the organic layer was washed with brine, dried over magnesium sulfate, and evaporated under reduced pressure. The obtained benzoic acid was diluted with 6N hydrogen chloride in 1,4-dioxane (10 ml) and the mixture was allowed to keep at room temperature for 4 hours. The mixture was evaporated under reduced pressure and the obtained solid was washed with ether to give 4-[[[(7S)-7-[[(2R)-2-(3-chlorophenyl)-2-hydroxyethyl]-amino]-5,6,7,8-tetrahydro-2-naphthalenyl]oxy]methyl]benzoic acid hydrochloride (87 mg).

NMR (200 MHz, DMSO-d₆, δ): 1.8-2.2 (2H, m), 2.6-3.6 (7H, m), 5.05 (1H, m), 5.16 (2H, s), 6.36 (1H, m), 6.7-7.0 (3H, m), 7.2-7.7 (6H, m), 7.95 (2H, d, J=8.4 Hz), 8.92 (1H, m), 9.33 (1H, m)

MS (m/z): 452 (M+1)

### EXAMPLE 78

The following compound was obtained according to a similar manner to that of Example 77.

### 3-[[[(7S)-7-[[(2R)-2-(3-Chlorophenyl)-2-hydroxyethyl]-amino]-5,6,7,8-tetrahydro-2-naphthalenyl]oxy]methyl]benzoic Acid Hydrochloride

NMR (200 MHz, DMSO-d₆, δ): 1.8-2.2 (2H, m), 2.6-3.6 (7H, m), 5.02 (1H, m), 5.14 (2H, s), 6.36 (1H, m), 6.7-7.0 (3H, m), 7.2-7.6 (5H, m), 7.66 (1H, d, J=8.4 Hz), 7.89 (1H, d, J=8.4 Hz), 7.99 (1H, s)

MS (m/z): 452 (M+1)

### EXAMPLE 79

To a mixture of (7S)-7-[N-[(2R)-2-(3-chlorophenyl)-2-hydroxyethyl]-N-(tert-butoxycarbonyl)amino]-2-bromomethyl-5,6,7,8-tetrahydronaphthalene (120 mg) in N,N-dimethylformamide (10 ml) were added ethyl 4-piperidinecarbonate (100 mg) and potassium carbonate (100 mg) at room temperature, and the mixture was stirred at the same temperature for 12 hours. The residue was diluted with a mixture of ethyl acetate and water, and the organic layer was washed with brine, dried over magnesium sulfate, and evaporated under reduced pressure. The residue was purified by column chromatography on silica gel to give ester derivative. To a solution of the obtained ester derivative in methanol (10 ml) was added 1N sodium hydroxide (5 ml) at room temperature, and the mixture was stirred at the same temperature for 12 hours. The resulting mixture was evaporated under reduced pressure. The residue was diluted with a mixture of ethyl acetate (30 ml) and 1N hydrochloric acid (10 ml), and the organic layer was washed with brine, dried over magnesium sulfate, and evaporated under reduced pressure. The obtained product was diluted with 6N hydrogen chloride in 1,4-dioxane (10 ml) and the mixture was allowed to keep at room temperature for 4 hours. The mixture was evaporated under reduced pressure and the obtained solid was washed with ether to give 1-[[(7S)-7-[[(2R)-2-(3-chlorophenyl)-2-hydroxyethyl]amino]-5,6,7,8-tetrahydro-2-naphthalenyl]methyl]-4-piperidinecarboxylic acid dihydrochloride (90 mg).

NMR (200 MHz, DMSO-d₆, δ): 1.8-3.8 (15H, m), 4.16 (2H, m), 5.08 (1H, m), 6.37 (1H, m), 7.0-7.7 (7H, m)

MS (m/z): 441 (M−1)

### EXAMPLE 80

The following compounds were obtained according to a similar manner to that of Example 79.

### (1) (3R)-1-[[(7S)-7-[[(2R)-2-(3-Chlorophenyl)-2-hydroxyethyl]amino]-5,6,7,8-tetrahydro-2-naphthalenyl]-methyl]-3-piperidinecarboxylic Acid Dihydrochloride

NMR (200 MHz, DMSO-d₆, δ): 1.8-3.8 (15H, m), 4.21 (2H, m), 5.08 (1H, m), 6.37 (1H, m), 7.0-7.5 (7H, m)

MS (m/z): 441 (M−1)

### (2) (3R)-1-[[(7S)-7-[[(2R)-2-(3-Chlorophenyl)-2-hydroxyethyl]amino]-5,6,7,8-tetrahydro-2-naphthalenyl]-methyl]-3-piperidinecarboxylic Acid Dihydrochloride

NMR (200 MHz, DMSO-d₆, δ): 1.8-3.8 (15H, m), 4.21 (2H, m), 5.08 (1H, m), 6.37 (1H, m), 7.0-7.5 (7H, m)

MS (m/z): 441 (M−1)

### EXAMPLE 81

The following compound was obtained according to a similar manner to that of Preparation 4.

### 3-[[(7S)-7-[[(2R)-2-(3-Chlorophenyl)-2-hydroxyethyl]-amino]-5,6,7,8-tetrahydro-2-naphthalenyl]methyl]benzoic Acid Hydrochloride

NMR (200 MHz, DMSO-d₆, δ): 1.8-2.2 (4H, m), 2.6-3.5 (5H, m), 5.02 (1H, m), 6.5-7.8 (11H, m)

MS (m/z): 436 (M+1)

1. A compound of the formula [I]: 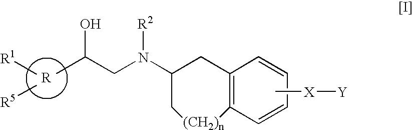
wherein 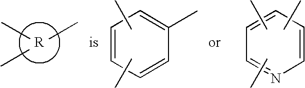
R¹ and R⁵ are each independently hydrogen, halogen, lower alkyl, mono(or di or tri)halo(lower)alkyl or cyano,
R² is hydrogen or an amino protective group,
X is bond, —O—, —O—CH₂—, 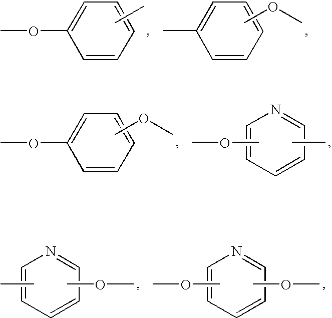
—(CH₂)_{q}— (in which q is 1 to 3),
—CH═CH—, —C≡C—, —NH—, —S— or —SO₂—,
Y is 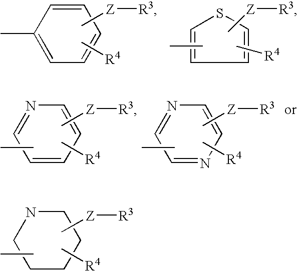
in which Z is bond, —O—(CH₂)ₘ— (in which m is 1 to 4), lower alkylene or lower alkenylene,
R³ is lower alkanoyl, carboxy, lower alkoxycarbonyl, carbamoyl, (lower alkylsulfonyl)carbamoyl, (phenylsulfonyl)carbamoyl, (benzylsulfonyl)carbamoyl or tetrazolyl, and
R⁴ is hydrogen, halogen, hydroxy, phenoxy, lower alkyl, lower alkoxy, cyclo(lower)alkyloxy, 3,4,5,6-tetrahydro-2H-pyranyloxy, phenoxy, nitro, cyano or 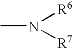
in which
R⁶ is hydrogen or lower alkyl, and
R⁷ is hydrogen, lower alkyl, lower alkanoyl, lower alkoxycarbonyl, benzyloxycarbonyl, benzoyl, furoyl, lower alkylcarbamoyl, phenylcarbamoyl, lower alkylsulfonyl, 3,4,5,6-tetrahydro-2H-pyranyl or phenyl, or
R⁶ and R⁷ are combined to form pyrrolidino or piperidino together with the nitrogen atom which may be substituted with oxo, and
n is 0, 1 or 2,
or a salt thereof.

2. A compound of claim 1, wherein
R¹ is hydrogen or halogen,
R² is hydrogen,
X is bond, —O—, —O—CH₂—, 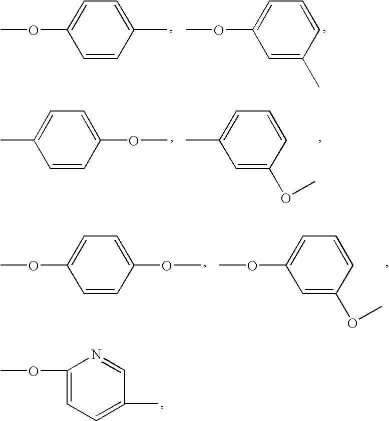
(CH₂)_{q}— (in which q is 1 or 2),
—CH═CH—, —C≡C—, —NH—, —S— or —SO₂—,
Y is 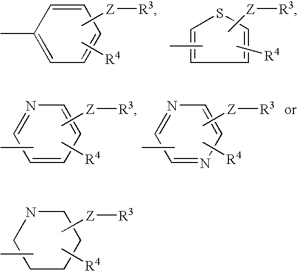
in which Z is bond, —O—(CH₂)ₘ— (in which m is 1 to 4), lower alkylene or lower alkenylene,
R³ is lower alkanoyl, carboxy, lower alkoxycarbonyl, carbamoyl, (lower alkylsulfonyl)carbamoyl, (phenylsulfonyl)carbamoyl, (benzylsulfonyl)carbamoyl or tetrazolyl, and
R⁴ is hydrogen, halogen, hydroxy, phenoxy, lower alkyl, lower alkoxy, cyclo(lower)alkyloxy, 3,4,5,6-tetrahydro-2H-pyranyloxy, phenoxy, nitro, cyano or 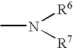
in which
R⁶ is hydrogen or lower alkyl, and
R⁷ is hydrogen, lower alkyl, lower alkanoyl, lower alkoxycarbonyl, benzyloxycarbonyl, benzoyl furoyl, lower alkylcarbamoyl, phenylcarbamoyl, lower alkylsulfonyl, 3,4,5,6-tetrahydro-2H-pyranyl or phenyl, or
R⁶ and R⁷ are combined to form pyrrolidino or piperidino together with the nitrogen atom which may be substituted with oxo, and
n is 0, 1 or 2.

3. A compound of claim 2, wherein 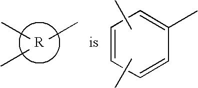
R¹ is halogen,
R⁵ is hydrogen,
R² is hydrogen,
X is bond, —O— or —O—CH₂—,
Y is 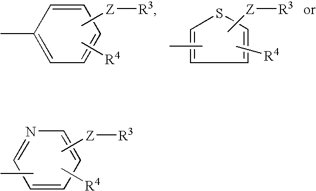
in which Z is bond, —O—(CH₂)ₘ— (in which m is 1 or 2) or lower alkenylene,
R³ is lower alkanoyl, carboxy, lower alkoxycarbonyl, carbamoyl or tetrazolyl, and
R⁴ is hydrogen or lower alkoxy, and
n is 1 or 2.

4. A compound of claim 3, wherein
R¹ is chloro,
X is bond or —O—,
Y is 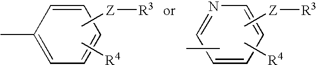
in which Z is bond or lower alkenylene,
R³ is carboxy, and
R⁴ is hydrogen or lower alkoxy, and
n is 1.

5. A compound of claim 4, which is
(1) 3-[[(7S)-7-[[(2R)-2-(3-Chlorophenyl)-2-hydroxyethyl]-amino]-5,6,7,8-tetrahydro-2-naphthalenyl]oxy]benzoic acid;
(2) 2-[[(7S)-7-[[(2R)-2-(3-Chlorophenyl)-2-hydroxyethyl]-amino]-5,6,7,8-tetrahydro-2-naphthalenyl]oxy]nicotinic acid;
(3) 3-[2-[[(7S)-7-[[(2R)-2-(3-Chlorophenyl)-2-hydroxyethyl]amino]-5,6,7,8-tetrahydro-2-naphthalenyl]-oxy]-3-pyridyl]-2-propenoic acid;
(4) 3-[6-[[(7S)-7-[[(2R)-2-(3-Chlorophenyl)-2-hydroxyethyl]amino]-5,6,7,8-tetrahydro-2-naphthalenyl]-oxy]-3-pyridyl]-2-propenoic acid;
(5) 4-[(7S)-7-[[(2R)-2-(4-Chlorophenyl)-2-hydroxyethyl]-amino]-5,6,7,8-tetrahydro-2-naphthalenyl]benzoic acid;
(6) 4-[(7S)-7-[[(2R)-2-(4-Chlorophenyl)-2-hydroxyethyl]-amino]-5,6,7,8-tetrahydro-2-naphthalenyl]-2-methoxybenzoic acid; or
(7) 5-[[(7S)-7-[[(2R)-2-(4-Chlorophenyl)-2-hydroxyethyl]-amino]-5,6,7,8-tetrahydro-2-naphthalenyl]oxy]-2-methoxybenzoic acid,
or a salt thereof.

6. A process for preparing a compound of claim 1,
or a salt thereof,
which comprises,
(i) reacting a compound [II] of the formula: 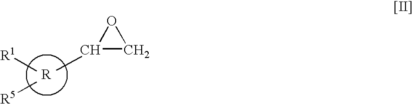
wherein 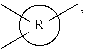
R¹ and R⁵ are each as defined in claim 1, with a compound [III] of the formula: 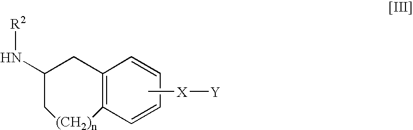
wherein R², X, Y and n are each as defined in claim 1, or a salt thereof, to give a compound [I] of the formula: 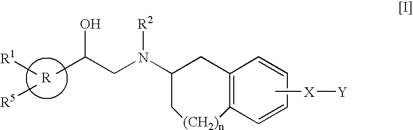
wherein 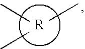
R¹, R⁵, R², X, Y and n are each as defined in claim 1,
or a salt thereof,
(ii) subjecting a compound [Ia] of the formula 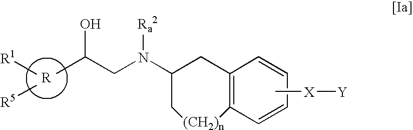
Wherein 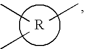
R¹, R⁵, X, Y and n are each as defined in claim 1, and
Rₐ² is an amino protective group,
or a salt thereof, to elimination reaction of the amino protective group, to give a compound [Ib] of the formula: 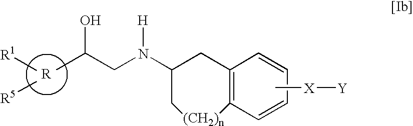
wherein 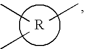
R¹, R⁵, X and Y are each as defined in claim 1,
or a salt thereof,
(iii) reacting a compound [IV] of the formula: 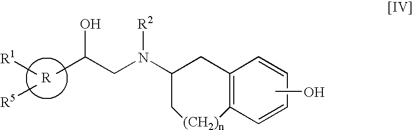
wherein 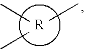
R¹, R⁵, R² and n are each as defined in claim 1,
or a salt thereof, with a compound [V] of the formula:
(HO)₂B—Y [V]
wherein Y is as defined in claim 1,
or a salt thereof, to give a compound [Ic] of the formula: 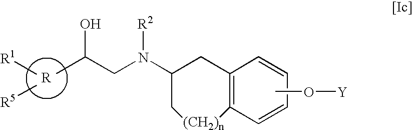
wherein 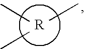
R¹, R⁵, R², Y and n are each as defined in claim 1,
or a salt thereof,
(iv) reacting a compound [IV] of the formula: 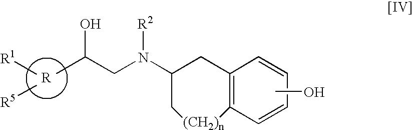
wherein 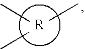
R¹, R⁵, R² and n are each as defined in claim 1,
or a salt thereof, with a compound [VI] of the formula:
X₁—Y [VI]
wherein Y is as defined in claim 1, and
X₁ is a leaving group,
or a salt thereof, to give a compound [IC] of the formula: 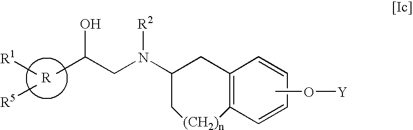
wherein 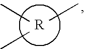
R¹, R⁵, R², Y and n are each as defined in claim 1,
or a salt thereof, and
(v) reacting a compound [VII] of the formula: 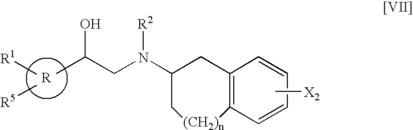
wherein 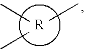
R¹, R⁵, R² and n are each as defined in claim 1,
X₂ is a leaving group,
or a salt thereof, with a compound [V] of the formula:
(HO)₂B—Y [V]
wherein Y is as defined in claim 1,
or a salt thereof, to give a compound [Id] of the formula: 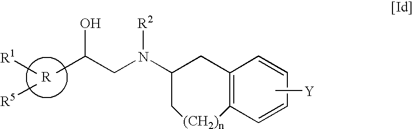
wherein 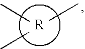
R¹, R¹, R⁵, R² Y and n are each as defined in claim 1,
or a salt thereof.

7. A pharmaceutical composition which comprises, as an active ingredient, a compound of claim 1 or a pharmaceutically acceptable salt thereof in admixture with pharmaceutically acceptable carriers or excipients.

8. Use of a compound of claim 1 or a pharmaceutically acceptable salt thereof for the manufacture of a medicament.

9. A compound of claim 1 or a pharmaceutically acceptable salt thereof for use as a medicament.

10. A compound of claim 1 or a pharmaceutically acceptable salt thereof for use as selective β₃ adrenergic receptor agonists.

11. A method for the prophylactic and/or the therapeutic treatment of pollakiuria, urinary incontinence, obesity or diabetes, which comprises administering a compound of claim 1 or a pharmaceutically acceptable salt thereof to a human being or an animal.
